(19) United States
(12) Patent Application Publication
(10) Pub. No.: US 2014/0220052 A1
(43) Pub. Date: Aug. 7, 2014

(54) COMPOSITIONS AND METHODS FOR WOUND HEALING, AND FOR RECRUITMENT AND ACTIVATION OF MACROPHAGES IN INJURED TISSUES AND IN IMPLANTED BIOMATERIALS USED FOR TISSUE ENGINEERING

(71) Applicant: University of Massachusetts, Boston, MA (US)

(72) Inventor: Uri Galili, Shrewsbury, MA (US)

(73) Assignee: University of Massachusetts

(21) Appl. No.: 14/226,487

(22) Filed: Mar. 26, 2014

Related U.S. Application Data
(63) Continuation-in-part of application No. 13/390,292, filed on May 1, 2012.
Application No. 13/390,292 is a continuation-in-part of application No. 12/542,377, filed on Aug. 17, 2009, now Pat. No. 8,084,057.
(60) Provisional application No. 61/853,099, filed on Mar. 28, 2013.

(51) Int. Cl. A61K 31/7016; A61K 47/48

(52) U.S. Cl. CPC A61K 31/7016; A61K 47/48246; A61K 47/48169
USPC 424/184.1

(57) ABSTRACT

The present invention is related to the field of wound healing or tissue regeneration due to disease (i.e., for example, cardiovascular diseases, osetoarthritic diseases, or diabetes) and to healing of internal injuries. In particular, the present invention provides compositions and methods comprising molecules and nanoparticles with linked α-gal epitopes for induction of recruitment and activation of macrophages localized within or surrounding damaged and/or injured tissue. The recruited macrophages further recruit stem cells into the injured tissues. The recruited macrophages and stem cells promote the repair and regeneration of the treated injured tissue. In some embodiments, the present invention provides treatments for tissue repair in normal subjects and in subjects having impaired healing capabilities, such as diabetic and aged subjects. In some embodiments, the present invention provides treatments for injured tissues such as brain, peripheral nerve, heart muscle, skeletal muscle, lung, cartilage, bone, gastrointestinal tract and dysfunctional endocrine tissues. The invention further provides methods and compositions comprising molecules and nanoparticles with linked α-gal epitopes for inducing recruitment and activation of macrophages into biomaterial implants for improving the conversion of such implants into functional tissues and organs within treated patients.

This application claims priority to U.S. provisional application Ser. No. 61/853,099, filed on Mar. 28, 2013, and is a continuation-in-part of, and claims priority to, co-pending U.S. patent application Ser. No. 13/390,292, filed on Feb. 13, 2012, which is the U.S. National stage filing of PCT Application No. PCT/US2010/45747, filed on Aug. 17, 2010, which is a continuation-in-part of, and claims priority to, U.S. patent application Ser. No. 12/542,377, filed on Aug. 17, 2009, that issued on Dec. 27, 2011 as U.S. Pat. No. 8,084,057, which is a continuation-in-part of, and claims priority to, PCT/US2008/008731, filed on Jul. 17, 2008, now abandoned, which claims priority under 35 U.S.C. §119(e) to U.S. provisional Patent Application Ser. No. 60/961,047, filed on Jul. 17, 2007, now abandoned, each of which is herein incorporated by reference in its entirety.

### FIELD OF INVENTION

The present invention is related to the field of wound healing and regeneration and repair of injured internal tissues and tissue engineering by synthetic implants and implants of natural origin. In particular, the present invention provides compositions and methods comprising molecules (such as nanoparticles) with linked α-gal epitopes for induction of an inflammatory response localized within or surrounding damaged tissue, and rapid localized recruitment and activation of macrophages that promote regeneration and repair of a wide variety of internal tissues and organs injured as a result of various types of trauma as well as of tissues and organs treated with biomaterials. In some embodiments, the present invention provides treatments for tissue repair and regeneration in normal subjects and in subjects having impaired healing capabilities, such as diabetic and aged subjects.

### BACKGROUND OF THE INVENTION

The inflammatory phase plays a critical role in wound healing regardless of the cause of the tissue damage. In addition to the destroying invading microbes, the inflammatory process is an integral part of the tissue repair process. Neutrophils are the first immune cells to arrive at the wound site where they phagocytose microbial agents and mediate wound debridement. Macrophages migrate into the wound several days post injury and become the predominant cell population before fibroblast migration and replication takes place. Compositions and methods to accelerate the pace and/or extent of wound and internal injury healing and regeneration are desirable, particularly in individuals with impaired healing capabilities, such as diabetic and aged individuals. Thus, there is a need for methods and compositions that promote healing in both external wounds and internal injuries.

### SUMMARY OF THE INVENTION

The present invention is related to the field of wound healing and tissue regeneration. In particular, the present invention provides compositions and methods comprising molecules with linked α-gal epitopes for induction of an inflammatory response localized within or surrounding damaged tissue and promotion of healing and repair of the injured tissue. In some embodiments, the present invention provides treatments for tissue repair in normal subjects and in subjects having impaired healing capabilities, such as diabetic and aged subjects.

In one embodiment, the present invention contemplates a method, comprising: a) providing; i) a subject having endogenous anti-Gal antibody, wherein the subject has an injured tissue; and ii) a preparation comprising an α-gal epitope having a terminal α-galactosyl; and b) applying said preparation to said tissue under conditions such that healing of said injured tissue is accelerated. In one embodiment, the tissue is an internal tissue. In one embodiment, the terminal α-galactosyl is selected from the group consisting of Galα1-3Gal, Galα1-2Gal, Galα1-6Gal, α-galactose sugar units capable of binding anti-Gal antibodies and α-gal epitope mimicking peptides linked to a macromolecule backbone or to another linker and they are capable of binding the anti-Gal antibody. In one embodiment, the α-gal epitope is soluble. In one embodiment, the α-gal epitope is attached to a molecule selected from the group consisting of a natural or synthetic glycolipid, glycoprotein, proteoglycan and a glycopolymer. In one embodiment, the preparation comprises α-gal liposomes and/or α-gal nanoparticles, i.e. α-gal liposomes that their size was decreased by sonication to a submicroscopic size in order to enable sterilization of the liposome suspension by filtration through a filter with pore size of 0.2 μm. In one embodiment, the α-gal liposomes further comprise anti-Gal antibodies. In one embodiment, the preparation further comprises an injury care device selected from, but not limited to, the group consisting of syringes, adhesive bands, compression bandages, wound dressings, sponges, gels, ointments, creams, suspensions, solutions, semi-permeable films, plasma clots, fibrin clots, biomaterials and processed allogeneic and xenogeneic tissues used for wound healing and tissue regeneration. In one embodiment, the device comprises physiological compositions including, but not limited to, solutions, suspensions, emulsions, creams, ointments, aerosol sprays, collagen containing substances, stabilizers, drops, matrix-forming substances, foams and/or dried preparation. In one embodiment, the preparation further comprises anti-Gal antibodies bound to said α-gal liposomes. In one embodiment, the injured tissue is selected from the group consisting of skin tissue brain tissue, nerve tissue, eye tissue, gastrointestinal tissue, muscle tissue, heart tissue, lung tissue, cartilage tissue, bone tissue, connective tissue, endocrine glands and/or vascular tissue. In one embodiment, the preparation comprises α-gal liposomes.

In one embodiment, the present invention contemplates a method, comprising: a) providing; i) a diabetic subject having endogenous anti-Gal antibody, wherein said subject has an injured pancreas such that insulin production is impaired; and ii) a preparation comprising an α-gal epitope having a terminal α-galactosyl; and b) applying said preparation to said pancreas, thereby creating regenerated Langerhans Islet cells. In one embodiment, the terminal α-galactosyl is selected from the group consisting of Galα1-3Gal, Galα1-2Gal, Galα1-6Gal, and α-galactose sugar units capable of binding anti-Gal antibodies. In one embodiment, the α-gal epitope is soluble. In one embodiment, the α-gal epitope is bound to a molecule selected from the group consisting of a natural or synthetic glycolipid, glycoprotein, and a glycopolymer. In one embodiment, the preparation comprises α-gal epitope mimicking peptides linked to a macromolecule backbone or to another linker and they are capable of binding the anti-Gal antibody. In one embodiment, the preparation further comprises α-gal liposomes. In one embodiment, the α-gal liposomes further comprise anti-Gal antibodies bound to α-gal liposomes. In one embodiment, the preparation further comprises an injury care device selected from the group consisting of syringes, adhesive bands, compression bandages, wound dressings, sponges, gels, ointments, creams, suspensions, solutions, semi-permeable films, plasma clots, fibrin clots and processed allogeneic and xenogeneic tissues used for wound healing and tissue regeneration. In one embodiment, the device comprises physiological compositions including, but not limited to, solutions, suspensions, emulsions, creams, ointments, aerosol sprays, collagen containing substances, stabilizers, drops, matrix-forming substances, foams and/or dried preparation. In one embodiment, the regenerated Langerhans Islet cells produce insulin.

In one embodiment, the present invention contemplates a method, comprising: a) providing; i) a subject having endogenous anti-Gal antibody and having an injured tissue selected from the group consisting of a peripheral nerve, a spinal cord, and a blood vessel. ii) a device comprising a biodegradable or non-biodegradable sheet comprising a preparation comprising an α-gal epitope having a terminal α-galactosyl; and b) wrapping said sheet around said injured tissue under conditions such that regeneration of said injured tissue is accelerated. In one embodiment, the terminal α-galactosyl is selected from the group consisting of Galα1-3Gal, Galα1-2Gal, Galα1-6Gal and any α-galactose sugar units capable of binding anti-Gal antibodies. In one embodiment, the α-gal epitope is soluble. In one embodiment, the α-gal epitope is bound to a molecule selected from the group consisting of a natural or synthetic glycolipid, glycoprotein, and a glycopolymer. In one embodiment, the preparation comprises α-gal epitope mimicking peptides linked to a macromolecule backbone or to another linker and they are capable of binding the anti-Gal antibody. In one embodiment, the preparation comprises α-gal liposomes. In one embodiment, the preparation further comprises anti-Gal antibodies bound to said α-gal liposomes. In one embodiment, the sheet is selected from the group consisting of a collagen sheet/or and a synthetic sheet.

In one embodiment, the present invention contemplates a method, comprising: a) providing: i) a subject having endogenous anti-Gal antibody; ii) damaged brain tissue; and iii) a preparation comprising an α-gal epitope having a terminal galactosyl; b) applying said preparation to said damaged brain tissue to produce treated brain tissue. In further embodiments, said terminal α-galactosyl is selected from the group consisting of Galα1-3Gal and Galα1-6Gal. In still further embodiments, said α-gal epitope is part of a molecule selected from the group consisting of a glycolipid, a glycoprotein, proteoglycan and a glycopolymer. In some embodiments, the preparation comprises α-gal epitope mimicking peptides linked to a macromolecule backbone or to another linker and they are capable of binding the anti-Gal antibody. In additional embodiments, said glycolipid comprises α-gal liposomes. In some embodiments, said applying is under conditions such that complement activation within or adjacent to said damaged tissue is enhanced. In further embodiments, said complement activation comprises production of C5a, C4a and C3a. In still further embodiments, said applying is under conditions such that neutrophil recruitment within or adjacent to said injured tissue is enhanced. In additional embodiments, said applying is under conditions such that monocyte and macrophage recruitment within or adjacent to said injured tissue is enhanced. In some embodiments, said applying is under conditions such that repair of said injured tissue is accelerated.

In one embodiment, the present invention contemplates a method, comprising: a) providing: i) a subject having endogenous anti-Gal antibody; ii) damaged skeletal muscle tissue; and iii) a preparation comprising an α-gal epitope having a terminal galactosyl; b) applying said preparation to said damaged skeletal muscle to produce treated skeletal muscle tissue. In further embodiments, said terminal α-galactosyl is selected from the group consisting of Galα1-3Gal and Galα1-6Gal. In still further embodiments, said α-gal epitope is part of a molecule selected from the group consisting of a glycolipid, a glycoprotein, proteoglycan and a glycopolymer. In some embodiments, the preparation comprises α-gal epitope mimicking peptides linked to a macromolecule backbone or to another linker and they are capable of binding the anti-Gal antibody. In additional embodiments, said glycolipid comprises α-gal liposomes. In some embodiments, said applying is under conditions such that complement activation within or adjacent to said damaged tissue is enhanced. In further embodiments, said complement activation comprises production of C5a, C4a and C3a. In still further embodiments, said applying is under conditions such that neutrophil recruitment within or adjacent to said injured tissue is enhanced. In additional embodiments, said applying is under conditions such that monocyte and macrophage recruitment within or adjacent to said injured tissue is enhanced. In some embodiments, said applying is under conditions such that repair of said injured tissue is accelerated.

In one embodiment, the present invention contemplates a method, comprising: a) providing: i) a subject having endogenous anti-Gal antibody; ii) damaged pancreatic tissue; and iii) a preparation comprising an α-gal epitope having a terminal galactosyl; b) applying said preparation to said damaged pancreatic tissue to produce treated pancreatic tissue. In further embodiments, said terminal α-galactosyl is selected from the group consisting of Galα1-3Gal and Galα1-6Gal. In still further embodiments, said α-gal epitope is part of a molecule selected from the group consisting of a glycolipid, a glycoprotein, proteoglycan and a glycopolymer. In additional embodiments, said glycolipid comprises α-gal liposomes. In some embodiments, the preparation comprises α-gal epitope mimicking peptides linked to a macromolecule backbone or to another linker and they are capable of binding the anti-Gal antibody. In some embodiments, said applying is under conditions such that complement activation within or adjacent to said damaged tissue is enhanced. In further embodiments, said complement activation comprises production of C5a, C4a and C3a. In still further embodiments, said applying is under conditions such that neutrophil recruitment within or adjacent to said injured tissue is enhanced. In additional embodiments, said applying is under conditions such that monocyte and macrophage recruitment within or adjacent to said injured tissue is enhanced. In some embodiments, said applying is under conditions such that repair of said injured tissue is accelerated.

In some embodiments, the present invention contemplates a method, comprising: a) providing: i) a subject having endogenous anti-Gal antibody; ii) damaged nerve tissue; and iii) a preparation comprising an α-gal epitope having a terminal galactosyl; b) applying said preparation to said damaged nerve tissue to produce treated nerve tissue. In further embodiments, said terminal α-galactosyl is selected from the group consisting of Galα1-3Gal and Galα1-6Gal. In still further embodiments, said α-gal epitope is part of a molecule selected from the group consisting of a glycolipid, a glycoprotein, proteoglycan and/or a glycopolymer. In additional embodiments, said glycolipid comprises α-gal liposomes. In one embodiment, the preparation comprises α-gal epitope mimicking peptides linked to a macromolecule backbone or to another linker and they are capable of binding the anti-Gal antibody. In some embodiments, said applying is under conditions such that complement activation within or adjacent to said damaged tissue is enhanced. In further embodiments, said complement activation comprises production of C5a, C4a and C3a. In still further embodiments, said applying is under conditions such that neutrophil recruitment within or adjacent to said injured tissue is enhanced. In additional embodiments, said applying is under conditions such that monocyte and macrophage recruitment within or adjacent to said injured tissue is enhanced. In some embodiments, said applying is under conditions such that repair of said injured tissue is accelerated.

In some embodiments, the present invention contemplates a method, comprising: a) providing: i) a subject having endogenous anti-Gal antibody; ii) damaged liver tissue; and iii) a preparation comprising an α-gal epitope having a terminal galactosyl; b) applying said preparation to said damaged liver tissue to produce treated liver tissue. In further embodiments, said terminal α-galactosyl is selected from the group consisting of Galα1-3Gal and Galα1-6Gal. In still further embodiments, said α-gal epitope is part of a molecule selected from the group consisting of a glycolipid, a glycoprotein, a proteoglycan and a glycopolymer. In additional embodiments, said glycolipid comprises α-gal liposomes. In some embodiments, the preparation comprises α-gal epitope mimicking peptides linked to a macromolecule backbone or to another linker and they are capable of binding the anti-Gal antibody. In some embodiments, said applying is under conditions such that complement activation within or adjacent to said damaged tissue is enhanced. In further embodiments, said complement activation comprises production of C5a, C4a and C3a. In still further embodiments, said applying is under conditions such that neutrophil recruitment within or adjacent to said injured tissue is enhanced. In additional embodiments, said applying is under conditions such that monocyte and macrophage recruitment within or adjacent to said injured tissue is enhanced. In some embodiments, said applying is under conditions such that repair of said injured tissue is accelerated.

In some embodiments, the present invention contemplates a method, comprising: a) providing: i) a subject having endogenous anti-Gal antibody; ii) damaged endocrine gland tissue; and iii) a preparation comprising an α-gal epitope having a terminal galactosyl; b) applying said preparation to said damaged endocrine gland tissue to produce treated endocrine gland tissue. In further embodiments, said terminal α-galactosyl is selected from the group consisting of Galα1-3Gal and Galα1-6Gal. In still further embodiments, said α-gal epitope is part of a molecule selected from the group consisting of a glycolipid, a glycoprotein, proteoglycan and/or a glycopolymer. In additional embodiments, said glycolipid comprises α-gal liposomes. In some embodiments, the preparation comprises α-gal epitope mimicking peptides linked to a macromolecule backbone or to another linker and they are capable of binding the anti-Gal antibody. In some embodiments, said applying is under conditions such that complement activation within or adjacent to said damaged tissue is enhanced. In further embodiments, said complement activation comprises production of C5a, C4a and C3a. In still further embodiments, said applying is under conditions such that neutrophil recruitment within or adjacent to said injured tissue is enhanced. In additional embodiments, said applying is under conditions such that monocyte and macrophage recruitment within or adjacent to said injured tissue is enhanced. In some embodiments, said applying is under conditions such that repair of said injured tissue is accelerated.

In some embodiments, the present invention contemplates a method, comprising: a) providing: i) a subject having endogenous anti-Gal antibody; ii) damaged bone tissue; and iii) a preparation comprising an α-gal epitope having a terminal galactosyl; b) applying said preparation to said damaged bone tissue to produce treated bone tissue. In further embodiments, said terminal α-galactosyl is selected from the group consisting of Galα1-3Gal and Galα1-6Gal. In still further embodiments, said α-gal epitope is part of a molecule selected from the group consisting of a glycolipid, a glycoprotein, proteoglycan and/or a glycopolymer. In additional embodiments, said glycolipid comprises α-gal liposomes. In some embodiments, the preparation comprises α-gal epitope mimicking peptides linked to a macromolecule backbone or to another linker and they are capable of binding the anti-Gal antibody. In some embodiments, said applying is under conditions such that complement activation within or adjacent to said damaged tissue is enhanced. In further embodiments, said complement activation comprises production of C5a, C4a and C3a. In still further embodiments, said applying is under conditions such that neutrophil recruitment within or adjacent to said injured tissue is enhanced. In additional embodiments, said applying is under conditions such that monocyte and macrophage recruitment within or adjacent to said injured tissue is enhanced. In some embodiments, said applying is under conditions such that repair of said injured tissue is accelerated.

In some embodiments, the present invention contemplates a method, comprising: a) providing: i) a subject having endogenous anti-Gal antibody; ii) damaged cartilage tissue; and iii) a preparation comprising an α-gal epitope having a terminal galactosyl; b) applying said preparation to said damaged cartilage tissue to produce treated cartilage tissue. In further embodiments, said terminal α-galactosyl is selected from the group consisting of Galα1-3Gal and Galα1-6Gal. In still further embodiments, said α-gal epitope is part of a molecule selected from the group consisting of a glycolipid, a glycoprotein, proteoglycan and/or a glycopolymer. In additional embodiments, said glycolipid comprises α-gal liposomes. In some embodiments, the preparation comprises α-gal epitope mimicking peptides linked to a macromolecule backbone or to another linker and they are capable of binding the anti-Gal antibody. In some embodiments, said applying is under conditions such that complement activation within or adjacent to said damaged tissue is enhanced. In further embodiments, said complement activation comprises production of C5a, C4a and C3a. In still further embodiments, said applying is under conditions such that neutrophil recruitment within or adjacent to said injured tissue is enhanced. In additional embodiments, said applying is under conditions such that monocyte and macrophage recruitment within or adjacent to said injured tissue is enhanced. In some embodiments, said applying is under conditions such that repair of said injured tissue is accelerated.

In one embodiment, the present invention contemplates a method, comprising: a) providing; i) a subject having endogenous anti-Gal antibody and an injured tissue; and ii) a preparation comprising an α-gal epitope having a terminal α-galactosyl as part of a tissue repair and regeneration preparation; and b) applying said preparation to said injury to produce a treated injured tissue. In one embodiment, the tissue is an internal tissue. In one embodiment, the terminal α-galactosyl is selected from the group consisting of Galα1-3Gal, Galα1-2Gal, Galα1-6Gal or any α-galactose sugar units capable of binding anti-Gal antibodies. In one embodiment, the α-gal epitope is free or part of a molecule selected from the group consisting of a natural or synthetic glycolipid, glycoprotein, proteoglycan and/or a glycopolymer. In additional embodiments, said glycolipid comprises α-gal liposomes. In one embodiment, the preparation comprises α-gal epitope mimicking peptides linked to a macromolecule backbone or to another linker and they are capable of binding the anti-Gal antibody. In one embodiment, the preparation further comprises an injury care device selected from the group consisting of syringes, adhesive bands, compression bandages, sponges, gels, semi-permeable films, plasma clots, fibrin clots. In one embodiment, the device comprises physiological compositions including, but not limited to, solutions, suspensions, emulsions, creams, ointments, aerosol sprays, collagen containing substances, stabilizers, drops, matrix-forming substances, foams and/or dried preparation. In one embodiment, the applying is under conditions such that complement activation within or adjacent to said injured tissue is enhanced. In one embodiment, the complement activation comprises production of complement fragments C5a, C4a and C3a. In one embodiment, the applying is under conditions such that neutrophil recruitment within or adjacent to said injury is enhanced. In one embodiment, the applying is under conditions such that monocyte and macrophage recruitment within or adjacent to said injured tissue is enhanced. In one embodiment, the applying is under conditions such that stem cell recruitment within or adjacent to said injury is enhanced. In one embodiment, the applying is under conditions such that injury healing and tissue repair and regeneration is accelerated.

In one embodiment, the present invention contemplates a method, comprising: a) providing; i) a subject having endogenous anti-Gal antibody and an injured tissue; and ii) a preparation comprising an α-gal liposomes having glycolipids and/or glycoproteins with a terminal α-galactosyl and comprising α-gal liposomes as part of a tissue repair and regeneration preparation, and/or α-gal epitope mimicking peptides linked to a macromolecule backbone or to another linker and they are capable of binding the anti-Gal antibody and comprising α-gal liposomes; and b) applying said preparation to said injury to produce a treated injured tissue. In one embodiment, the preparation further comprises an injury care device selected from the group consisting of syringes, adhesive bands, compression bandages, sponges, gels, semi-permeable films, plasma clots, fibrin clots. In one embodiment, the device comprises physiological compositions including, but not limited to, solutions, suspensions, emulsions, creams, ointments, aerosol sprays, collagen containing substances, stabilizers, drops, matrix-forming substances, foams and/or dried preparation. In one embodiment, the preparation further comprises anti-Gal antibodies bound to said α-gal liposomes. In one embodiment, the applying is under conditions such that complement activation within or adjacent to said injured tissue is enhanced. In one embodiment, the complement activation comprises production of complement fragments C5a, C4a and C3a. In one embodiment, the applying is under conditions such that neutrophil recruitment within or adjacent to said injured tissue is enhanced. In one embodiment, the applying is under conditions such that monocyte and macrophage recruitment within or adjacent to said injured tissue is enhanced. In one embodiment, the applying is under conditions such that stem cell recruitment within or adjacent to said injury is enhanced. In one embodiment, the applying is under conditions such that injury healing and tissue repair and regeneration is accelerated.

In one embodiment, the present invention contemplates a method, comprising: a) providing; i) a subject having endogenous anti-Gal antibody and one or more of an injured tissue including, but not limited to, brain tissue, nerve tissue, eye tissue, gastrointestinal tissue, muscle tissue, lung tissue, cartilage tissue, bone tissue, endocrine glands and vascular tissue; ii) a preparation comprising an α-gal epitope having a terminal α-galactosyl as part of a tissue repair and regeneration preparation; and b) applying said preparation to said injured tissue to produce a treated injured tissue. In one embodiment, the terminal α-galactosyl is selected from the group consisting of Galα1-3Gal, Galα1-2Gal, Galα1-6Gal and any α-galactose sugar units capable of binding anti-Gal antibodies. In one embodiment, the α-gal epitope is free or part of a molecule selected from the group consisting of a natural or synthetic glycolipid, glycoprotein, and/or a glycopolymer. In one embodiment, the preparation comprises α-gal liposomes. In one embodiment, the preparation comprises α-gal epitope mimicking peptides linked to a macromolecule backbone or to another linker and they are capable of binding the anti-Gal antibody. In one embodiment, the preparation further comprises an injury care device selected from the group consisting of syringes, adhesive bands, compression bandages, sponges, gels, semi-permeable films, plasma clots, fibrin clots. In one embodiment, the device comprises physiological compositions including, but not limited to, solutions, suspensions, emulsions, creams, ointments, aerosol sprays, collagen containing substances, stabilizers, drops, matrix-forming substances, foams and/or dried preparation. In one embodiment, the preparation further comprises anti-Gal antibodies bound to said α-gal liposomes. In one embodiment, the applying is under conditions such that complement activation within or adjacent to said injured tissue is enhanced. In one embodiment, the complement activation comprises production of complement fragments C5a, C4a and C3a. In one embodiment, the applying is under conditions such that neutrophil recruitment within or adjacent to said injured tissue is enhanced. In one embodiment, the applying is under conditions such that monocyte and macrophage recruitment within or adjacent to said injured tissue is enhanced. In one embodiment, the applying is under conditions such that stem cell recruitment within or adjacent to said injury is enhanced. In one embodiment, the applying is under conditions such that injury healing and tissue repair and regeneration is accelerated.

In one embodiment, the present invention contemplates a method, comprising: a) providing; i) a subject having endogenous anti-Gal antibody and having diabetes in which insulin production is impaired; and ii) a preparation comprising an α-gal epitope having a terminal α-galactosyl as part of a tissue repair and regeneration preparation; and b) applying said preparation into the pancreas of said subject to induce regeneration of Langerhans Islets and production of endogenous insulin. In one embodiment, the terminal α-galactosyl is selected from the group consisting of Galα1-3Gal, Galα1-2Gal, Galα1-6Gal and any α-galactose sugar units capable of binding anti-Gal antibodies. In one embodiment, the α-gal epitope is free or part of a molecule selected from the group consisting of a natural or synthetic glycolipid, glycoprotein, and/or a glycopolymer. In one embodiment, the preparation comprises α-gal liposomes. In one embodiment, the preparation comprises α-gal epitope mimicking peptides linked to a macromolecule backbone or to another linker and they are capable of binding the anti-Gal antibody. In one embodiment, the preparation further comprises an injury care device selected from the group consisting of syringes, adhesive bands, compression bandages, sponges, gels, semi-permeable films, plasma clots, fibrin clots. In one embodiment, the device comprises physiological compositions including, but not limited to, solutions, suspensions, emulsions, creams, ointments, aerosol sprays, collagen containing substances, stabilizers, drops, matrix-forming substances, foams and/or dried preparation. In one embodiment, the preparation further comprises anti-Gal antibodies bound to said α-gal liposomes. In one embodiment, the applying is under conditions such that complement activation within or adjacent to said injured tissue is enhanced. In one embodiment, the complement activation comprises production of complement fragments C5a, C4a and C3a. In one embodiment, the applying is under conditions such that neutrophil recruitment within pancreas is enhanced. In one embodiment, the applying is under conditions such that monocyte and macrophage recruitment pancreas is enhanced. In one embodiment, the applying is under conditions such that stem cell recruitment within pancreas is enhanced. In one embodiment, the recruited stem cells differentiate into Langerhans Islet cells. In one embodiment, the Langerhans Islet cells produce insulin.

In one embodiment, the present invention contemplates a method, comprising a) providing; i) a subject having endogenous anti-Gal antibody and having injury in a peripheral nerve, spinal cord, blood vessel or any other tissue: ii) a device comprising a biodegradable or non-biodegradable sheet coated with or containing a preparation comprising an α-gal epitope having a terminal α-galactosyl as part of a tissue repair and regeneration preparation; and b) applying said sheet around said injured nerve, spinal cord, blood vessel, or other tissue to produce a treated injured tissue. In one embodiment, the terminal α-galactosyl is selected from the group consisting of Galα1-3Gal, Galα1-2Gal, Galα1-6Gal and any α-galactose sugar units capable of binding anti-Gal antibodies. In one embodiment, the α-gal epitope is free or part of a molecule selected from the group consisting of a natural or synthetic glycolipid, glycoprotein, and/or a glycopolymer. In one embodiment, the preparation comprises α-gal liposomes. In one embodiment, the preparation comprises α-gal epitope mimicking peptides linked to a macromolecule backbone or to another linker and they are capable of binding the anti-Gal antibody. In one embodiment, the preparation is part of a injury care device selected from the group consisting of collagen containing sheet, synthetic sheet, or any other sheet that can be wrapped around the injured nerve, spinal cord, blood vessel, or other injured tissue. In one embodiment, the preparation further comprises anti-Gal antibodies bound to said α-gal liposomes. In one embodiment, the applying is under conditions such that complement activation within or adjacent to said injured tissue is enhanced. In one embodiment, the complement activation comprises production of complement fragments C5a, C4a and C3a. In one embodiment, the applying is under conditions such that neutrophil recruitment to the injured tissue is enhanced. In one embodiment, the applying is under conditions such that monocyte and macrophage recruitment to the injured tissue is enhanced. In one embodiment, the applying is under conditions such that stem cell recruitment to the injured tissue is enhanced. In one embodiment, the recruited stem cells differentiate into cells that repair the injured tissue.

In some embodiments, the invention relates to a method, comprising: a) providing: i) a subject having endogenous anti-Gal antibody; ii) a wound; and iii) a preparation comprising an α-gal epitope having a terminal galactosyl; and b) applying said preparation to said wound to produce a treated wound. In further embodiments, said terminal α-galactosyl is selected from the group consisting of Galα1-3Gal, and Galα1-6Gal. In still further embodiments, said α-gal epitope is part of a molecule selected from the group consisting of a glycolipid, a glycoprotein, and/or a glycopolymer. In additional embodiments, said glycolipid comprises α-gal liposomes. In one embodiment, the preparation comprises α-gal epitope mimicking peptides linked to a macromolecule backbone or to another linker and they are capable of binding the anti-Gal antibody. In some embodiments, said applying is under conditions such that complement activation within or adjacent to said wound is enhanced. In further embodiments, said complement activation comprises production of C5a, C4a and C3a. In still further embodiments, said applying is under conditions such that neutrophil recruitment within or adjacent to said wound is enhanced. In additional embodiments, said applying is under conditions such that monocyte and macrophage recruitment within or adjacent to said wound is enhanced. In some embodiments, said applying is under conditions such that wound closure is accelerated. In further embodiments, the method is used to treat subjects diagnosed with or exhibiting symptoms associated with heart disease and damage, arthritis, osetoarthritis, cartilage repair and diabetes mellitus. In still further embodiments, the disclosed method is used to treat tissue or organ damage in combination with the application of stem cells.

In some embodiments the invention relates to a method, comprising: a) providing; i) a subject having a wound; ii) a wound care device comprising a preparation comprising an α-gal epitope having a terminal α-galactosyl, and iii) an anti-Gal antibody; and b) applying said wound care device to said wound to produce a treated wound. In further embodiments, said terminal α-galactosyl is selected from the group consisting of Galα1-3Gal, Galα1-2Gal and Galα1-6Gal. In still further embodiments, said α-gal epitope is part of a molecule selected from the group consisting of a glycolipid, a glycoprotein, and/or a glycopolymer. In additional embodiments, said glycolipid comprises α-gal liposomes. In one embodiment, the preparation comprises α-gal epitope mimicking peptides linked to a macromolecule backbone or to another linker and they are capable of binding the anti-Gal antibody. In some embodiments, said preparation is part of a wound care device selected from the group consisting of adhesive bands, compression bandages, gels, semi-permeable films, and foams. In further embodiments, the disclosed method and preparation is used to treat subjects diagnosed with or exhibiting symptoms associated with heart disease and damage, arthritis, osetoarthritis, cartilage repair and diabetes mellitus. In still further embodiments, the disclosed method and preparation is used to treat tissue or organ damage in combination with the application of stem cells.

In some embodiments, the invention relates to a burn care device comprising a preparation comprising an α-gal epitope having a terminal α-galactosyl. In further embodiments, said terminal α-galactosyl is selected from the group consisting of Galα1-3Gal, and Galα1-6Gal. In still further embodiments, said α-gal epitope is part of a molecule selected from the group consisting of a glycolipid, a glycoprotein, and/or a glycopolymer. In additional embodiments, said glycolipid comprises α-gal liposomes. In one embodiment, the preparation comprises α-gal epitope mimicking peptides linked to a macromolecule backbone or to another linker and they are capable of binding the anti-Gal antibody. In some embodiments, said preparation further comprises anti-Gal antibodies bound to said α-gal liposomes. In further embodiments, said device is in the form of one of the group consisting of adhesive bands, compression bandages, gels, semipermeable films, and foams. In further embodiments, the disclosed device and preparation is used to treat subjects diagnosed with or exhibiting symptoms associated with heart disease and damage, arthritis, osetoarthritis, cartilage repair and diabetes mellitus. In still further embodiments, the disclosed device and preparation is used to treat tissue or organ damage in combination with the application of stem cells.

In some embodiments, the invention relates to a method, comprising: a) providing: i) a subject having endogenous anti-Gal antibody; and ii) damaged cardiac tissue; and iii) a preparation comprising an α-gal epitope having a terminal galactosyl; b) applying said preparation to said damaged cardiac tissue to produce treated cardiac tissue. In further embodiments, said terminal α-galactosyl is selected from the group consisting of Galα1-3Gal, Galα1-2Gal and Galα1-6Gal. In still further embodiments, said α-gal epitope is part of a molecule selected from the group consisting of a glycolipid, a glycoprotein, and a glycopolymer. In additional embodiments, said glycolipid comprises α-gal liposomes. In some embodiments, the preparation comprises α-gal epitope mimicking peptides linked to a macromolecule backbone or to another linker and they are capable of binding the anti-Gal antibody. In some embodiments, said applying is under conditions such that complement activation within or adjacent to said damaged cardiac tissue is enhanced. In further embodiments, said complement activation comprises production of C5a, C4a and C3a. In still further embodiments, said applying is under conditions such that neutrophil recruitment within or adjacent to said damaged cardiac tissue is enhanced. In additional embodiments, said applying is under conditions such that monocyte and macrophage recruitment within or adjacent to said damaged cardiac tissue is enhanced. In one embodiment, the applying is under conditions such that stem cell recruitment within or adjacent to said injury is enhanced. In some embodiments, said applying is under conditions such that repair of said damaged cardiac tissue is accelerated.

In some embodiments, the invention relates to a method, comprising: providing a subject having endogenous anti-Gal antibody and tissue damaged by diabetes; and a preparation comprising an α-gal epitope having a terminal galactosyl; and applying said preparation to said tissue damaged by diabetes to produce treated tissue. In further embodiments, said terminal α-galactosyl is selected from the group consisting of Galα1-3Gal, Galα1-2Gal, and Galα1-6Gal. In still further embodiments, said α-gal epitope is part of a molecule selected from the group consisting of a glycolipid, a glycoprotein, and/or a glycopolymer. In additional embodiments, said glycolipid comprises α-gal liposomes. In some embodiments, the preparation comprises α-gal epitope mimicking peptides linked to a macromolecule backbone or to another linker and they are capable of binding the anti-Gal antibody. In some embodiments, said applying is under conditions such that complement activation within or adjacent to said tissue damaged by diabetes is enhanced. In further embodiments, said complement activation comprises production of C5a and C3a. In still further embodiments, said applying is under conditions such that neutrophil recruitment within or adjacent to said tissue damaged by diabetes is enhanced. In additional embodiments, said applying is under conditions such that monocyte and macrophage recruitment within or adjacent to said tissue damaged by diabetes is enhanced. In some embodiments, said applying is under conditions such that repair of said tissue damaged by diabetes is accelerated.

In some embodiments, the invention relates to a method, comprising: providing a subject having endogenous anti-Gal antibody and tissue damaged by osteoarthritis; and a preparation comprising an α-gal epitope having a terminal galactosyl; and applying said preparation to said tissue damaged by osteoarthritis to produce treated tissue. In further embodiments, said terminal α-galactosyl is selected from the group consisting of Galα1-3Gal, Galα1-2Gal and Galα1-6Gal. In still further embodiments, said α-gal epitope is part of a molecule selected from the group consisting of a glycolipid, a glycoprotein, and/or a glycopolymer. In additional embodiments, said glycolipid comprises α-gal liposomes. In some embodiments, the preparation comprises α-gal epitope mimicking peptides linked to a macromolecule backbone or to another linker and they are capable of binding the anti-Gal antibody. In some embodiments, said applying is under conditions such that complement activation within or adjacent to said tissue damaged by osteoarthritis is enhanced. In further embodiments, said complement activation comprises production of C5a, C4a and C3a. In still further embodiments, said applying is under conditions such that neutrophil recruitment within or adjacent to said tissue damaged by osteoarthritis is enhanced. In additional embodiments, said applying is under conditions such that monocyte and macrophage recruitment within or adjacent to said tissue damaged by osteoarthritis is enhanced. In some embodiments, said applying is under conditions such that repair of said tissue damaged by osteoarthritis is accelerated. In further embodiments, said tissue damaged by osteoarthritis is selected from the group consisting of bone and cartilage.

In one embodiment, the present invention contemplates a method, comprising: a) providing; i) a subject having endogenous anti-Gal antibody, wherein the subject has an injured tissue and wherein the injured tissue is capable of forming a scar; and ii) a preparation comprising an α-gal epitope having a terminal α-galactosyl; and b) applying said preparation to said tissue under conditions such that the scar formation is prevented. In one embodiment, the tissue is an internal tissue. In one embodiment, the terminal α-galactosyl is selected from the group consisting of Galα1-3Gal, Galα1-2Gal, Galα1-6Gal, α-galactose sugar units capable of binding anti-Gal antibodies and α-gal epitope mimicking peptides linked to a macromolecule backbone or to another linker and they are capable of binding the anti-Gal antibody. In one embodiment, the α-gal epitope is soluble. In one embodiment, the α-gal epitope is attached to a molecule selected from the group consisting of a natural or synthetic glycolipid, glycoprotein, proteoglycan and/or a glycopolymer. In one embodiment, the preparation comprises α-gal liposomes. In one embodiment, the α-gal liposomes further comprise anti-Gal antibodies. In one embodiment, the preparation further comprises an injury care device selected from, but not limited to, the group consisting of syringes, adhesive bands, compression bandages, wound dressings, sponges, gels, ointments, creams, suspensions, solutions, semi-permeable films, plasma clots, fibrin clots. In one embodiment, the device comprises physiological compositions including, but not limited to, solutions, suspensions, emulsions, creams, ointments, aerosol sprays, collagen containing substances, stabilizers, drops, matrix-forming substances, foams and/or dried preparation. In one embodiment, the preparation further comprises anti-Gal antibodies bound to said α-gal liposomes. In one embodiment, the injured tissue is selected from the group consisting of skin tissue brain tissue, nerve tissue, eye tissue, gastrointestinal tissue, muscle tissue, heart tissue, lung tissue, cartilage tissue, bone tissue, connective tissue, endocrine glands and/or vascular tissue. In one embodiment, the preparation comprises α-gal liposomes.

In one embodiment, the present invention contemplates a method, comprising: a) providing; i) a subject comprising an ischemic heart muscle caused by a myocardial infarction, wherein the subject further comprises endogenous anti-Gal antibody; ii) a preparation comprising an α-gal epitope having a terminal α-galactosyl; and b) applying said preparation to said ischemic heart muscle, thereby creating regenerated heart muscle cells in the ischemic tissue. In one embodiment, the ischemic heart muscle comprises injured heart muscle. In one embodiment, the terminal α-galactosyl is selected from the group consisting of Galα1-3Gal, Galα1-2Gal, Galα1-6Gal, and α-galactose sugar units capable of binding anti-Gal antibodies. In one embodiment, the α-gal epitope is soluble. In one embodiment, the α-gal epitope is bound to a molecule selected from the group consisting of a natural or synthetic glycolipid, glycoprotein, and/or a glycopolymer. In one embodiment, the preparation further comprises of anti-Gal antibodies bound to said α-gal liposomes. In one embodiment, the preparation is administered into said injured heart muscle by injection. In one embodiment, the regenerated heart muscle cells partially or fully restore the contractile activity of the injured heart muscle.

The invention also provides method for inducing recruitment of macrophages into injured internal tissues and for activation of said recruited macrophages to produce pro-healing cytokines and growth factors in a subject having endogenous anti-Gal antibody, comprising administering to said injured internal tissue composition that comprises α-gal nanoparticles which are submicroscopic α-gal liposomes, wherein i) said α-gal nanoparticles comprises α-gal epitope having a terminal α-galactosyl and ii) said administering under conditions that increasing the amount of macrophages in said injured internal tissue of said subject. In one embodiment, the terminal α-galactosyl is selected from the group consisting of Galα1-3Gal, Galα1-2Gal, Galα1-6Gal or any α-galactose sugar units capable of binding anti-Gal antibodies. In one embodiment, the α-gal epitope is free or part of a molecule selected from the group consisting of a natural or synthetic glycolipid, glycoprotein, and a glycopolymer. In one embodiment, the α-gal nanoparticles are applied to injured internal tissues including: heart muscle, skeletal muscle, smooth muscle, connective tissue, ligament, bone, nerve tissue, brain, spinal cord, blood vessels, endocrine glands, exocrine glands, liver, kidney, gall bladder, thyroid, parathyroid, pancreas, esophagus, stomach, small intestine, large intestine, lung, trachea, bronchi, bronchioles, alveoli, eye, ear, ovary, testis, urinary bladder, skin. In one embodiment, the α-gal nanoparticles are applied to injured or severed fingers, toes, arms and feet. In one embodiment, the preparation is part of an injury care device selected from the group consisting of injections, adhesive bands, compression bandages, gels, semi-permeable films, plasma clots, fibrin clots, water, solutions, suspensions, emulsions, creams, ointments, aerosol sprays, collagen containing substances, stabilizers, sponges, drops, matrix-forming substances, foams or dried preparation. In one embodiment, the applying step is under conditions such that complement activation within or adjacent to said injured tissue is enhanced. In one embodiment, the complement activation comprises production of complement cleavage chemotactic peptides including C5a, C4a and C3a. In one embodiment, the applying step is under conditions such that monocyte and macrophage recruitment within or adjacent to said injured tissue is enhanced. In one embodiment, the applying step is under conditions such that stem cell recruitment within or adjacent to said injury is enhanced. In one embodiment, the applying step is under conditions such that injury healing and tissue repair and regeneration is induced or accelerated.

The invention also provides a method for inducing recruitment of macrophages into biomaterial implants for activation of said recruited macrophages to produce pro-healing cytokines and growth factors in a subject having endogenous anti-Gal antibody, comprising administering to said biomaterial composition that comprises α-gal nanoparticles, wherein

i) said α-gal nanoparticles comprises α-gal epitope having a terminal α-galactosyl and ii) said administering under conditions that increasing the amount of macrophages in said injured internal tissue of said subject. In one embodiment, the biomaterial is a natural tissue or organ selected from the group consisting of heart, urinary bladder, gall bladder, lung, trachea, bronchi, bronchioles, alveoli, skeletal muscle, smooth muscle, connective tissue, endocrine glands, exocrine glands, ligament, cartilage, bone, nerve tissue, brain, spinal cord, blood vessels, liver, kidney, thyroid, parathyroid, pancreas, esophagus, stomach, small intestine, large intestine, ovary, testis, eye, ear, and skin. In one embodiment, the biomaterial implant is comprised of collagen containing α-gal nanoparticles, cartilage fragments mixed with α-gal nanoparticles or bone fragments mixed with α-gal nanoparticles. In one embodiment, the biomaterial implant is dried or not dried and immersed in α-gal nanoparticles suspension for penetration of said α-gal nanoparticles into said biomaterial implant. In one embodiment, the biomaterial implant organ or tissue is perfused with α-gal nanoparticles suspension in order to introduce said α-gal nanoparticles into said biomaterial. In one embodiment, the anti-Gal antibodies are bound to said α-gal nanoparticles. In one embodiment, the applying step is under conditions such that complement activation within or adjacent to said biomaterial implant is enhanced. In one embodiment, the complement activation comprises production of complement fragments C5a, C4a and C3a. In one embodiment, the applying step is under conditions such that monocyte and macrophage recruitment within or adjacent to said biomaterial implant is enhanced. In one embodiment, the applying step is under conditions such that stem cell recruitment within or adjacent to said biomaterial implant is enhanced. In one embodiment, the biomaterial is a synthetic biomaterial.

The invention also provides a method for inducing recruitment of macrophages into tissues where stem cells are administered for activation of said recruited macrophages to produce cytokines and growth factors that support the activity of said stem cells in a subject having endogenous anti-Gal antibody, comprising administering to said tissue composition that comprises α-gal nanoparticles and stem cells, wherein i) said α-gal nanoparticles comprises α-gal epitope having a terminal α-galactosyl and ii) said administering under conditions that increasing the amount of macrophages in said administration site of said cells in said subject. In one embodiment, the terminal α-galactosyl is selected from the group consisting of Galα1-3Gal, Galα1-2Gal, Galα1-6Gal or any α-galactose sugar units capable of binding anti-Gal antibodies. In one embodiment, the α-gal epitope is free or part of a molecule selected from the group consisting of a natural or synthetic glycolipid, glycoprotein, and a glycopolymer. In one embodiment, the applying is under conditions such that complement activation within or adjacent to said injured tissue is enhanced. In one embodiment, the complement activation comprises production of complement cleavage chemotactic peptides including C5a, C4a and C3a. In one embodiment, the applying is under conditions such that monocyte and macrophage recruitment within or adjacent to said tissue is enhanced. In one embodiment, the administered stem cells are adult stem cells or embryonic stem cells. In one embodiment, the administered stem cells are mature cells that were converted into stem cells.

The invention further provides a method for inducing recruitment of macrophages into injured internal tissues and for activation of said recruited macrophages to produce pro-healing cytokines and growth factors in a subject having endogenous anti-Gal antibody, comprising administering to said injured internal tissue composition that comprises a soluble molecules with one or more α-gal epitopes, wherein i) said soluble α-gal carrying molecules comprise α-gal epitope having a terminal α-galactosyl and ii) said administering under conditions that increasing the amount of macrophages in said injured internal tissue of said subject. In one embodiment, the terminal α-galactosyl is selected from the group consisting of Galα1-3Gal, Galα1-2Gal, Galα1-6Gal or any α-galactose sugar units capable of binding anti-Gal antibodies. In one embodiment, the α-gal epitope is free or part of a molecule selected from the group consisting of a natural or synthetic glycolipid, glycoprotein, and/or a glycopolymer. In one embodiment, the soluble α-gal epitope carrying molecules are applied to injured internal tissues including: heart muscle, skeletal muscle, smooth muscle, connective tissue, ligament, bone, nerve tissue, brain, spinal cord, liver, kidney, thyroid, parathyroid, pancreas, esophagus, stomach, small intestine, large intestine, lung, trachea, bronchioles, alveoli, eye, ear, glands, blood vessels, ovary, testis and skin. In one embodiment, the soluble α-gal epitope carrying molecules are applied to injured or severed fingers, toes, arms and feet. In one embodiment, the soluble α-gal epitope carrying molecules are part of an injury care device selected from the group consisting of injections, adhesive bands, compression bandages, gels, semi-permeable films, plasma clots, fibrin clots, water, solutions, suspensions, emulsions, creams, ointments, aerosol sprays, collagen containing substances, stabilizers, sponges, drops, matrix-forming substances, foams or dried preparation. In one embodiment, the applying is under conditions such that complement activation within or adjacent to said injured tissue is enhanced. In one embodiment, the complement activation comprises production of complement fragments C5a, C4a and C3a. In one embodiment, the applying is under conditions such that monocyte and macrophage recruitment within or adjacent to said injured tissue is enhanced. In one embodiment, the applying is under conditions such that stem cell recruitment within or adjacent to said injury is enhanced. In one embodiment, the applying is under conditions such that injury healing and tissue repair and regeneration is accelerated.

Also provided by the invention is a method for inducing recruitment of macrophages into biomaterial implants for activation of said recruited macrophages to produce pro-healing cytokines and growth factors in a subject having endogenous anti-Gal antibody, comprising administering to said biomaterial composition that comprises soluble molecules with one or more α-gal epitopes, wherein i) said soluble α-gal carrying molecules comprise α-gal epitope having a terminal α-galactosyl and ii) said administering under conditions that increasing the amount of macrophages in said biomaterial implant of said subject. In one embodiment, the biomaterial is a natural tissue or organ selected from the group consisting of heart, urinary bladder, gall bladder, lung, trachea bronchi, bronchioles, alveoli, skeletal muscle, smooth muscle, connective tissue, ligament, cartilage, bone, nerve tissue, brain, spinal cord, liver, kidney, thyroid, parathyroid, pancreas, esophagus, stomach, small intestine, large intestine, eye, ear, and skin. In one embodiment, the biomaterial implant is comprised of collagen, cartilage fragments or bone fragments mixed with said soluble α-gal epitopes carrying molecules. In one embodiment, the biomaterial implant is dried or not dried and immersed in soluble α-gal epitopes carrying molecules suspension for penetration of said soluble α-gal epitopes carrying molecules into the biomaterial implant. In one embodiment, the biomaterial implant organ or tissue is perfused with soluble α-gal epitopes carrying molecules in order to introduce soluble α-gal epitopes carrying molecules into said biomaterial. In one embodiment, the anti-Gal antibodies are bound to said soluble α-gal epitopes carrying molecules. In one embodiment, the applying is under conditions such that complement activation within or adjacent to said biomaterial implant is enhanced. In one embodiment, the complement activation comprises production of complement fragments C5a, C4a and C3a. In one embodiment, the applying is under conditions such that monocyte and macrophage recruitment within or adjacent to said biomaterial implant is enhanced. In one embodiment, the applying is under conditions such that stem cell recruitment within or adjacent to said biomaterial implant is enhanced. In one embodiment, the biomaterial is a synthetic biomaterial.

The invention further provides a method for inducing recruitment of macrophages into biomaterial implants for activation of said recruited macrophages to produce pro-healing cytokines and growth factors in a subject having endogenous anti-Gal antibody, comprising administering to said biomaterial composition that comprises α-gal nanoparticles, wherein i) said α-gal nanoparticles comprises α-gal epitope having a terminal α-galactosyl and ii) said administering is under conditions for increasing the amount of macrophages in injured internal tissue of said subject. In one embodiment, said terminal α-galactosyl is selected from the group consisting of Galα1-3Gal, Galα1-2Gal, Galα1-6Gal or any α-galactose sugar units capable of binding anti-Gal antibodies. In one embodiment, said α-gal epitope is free or part of a molecule selected from the group consisting of a natural or synthetic glycolipid, glycoprotein, and a glycopolymer. In one embodiment, said biomaterial is a natural tissue or organ selected from the group consisting of heart, urinary bladder, gall bladder, lung, trachea, bronchi, bronchioles, alveoli, skeletal muscle, smooth muscle, connective tissue, endocrine glands, exocrine glands, ligament, cartilage, bone, nerve tissue, brain, spinal cord, blood vessels, liver, kidney, thyroid, parathyroid, pancreas, esophagus, stomach, small intestine, large intestine, ovary, testis, eye, ear, and skin. In one embodiment, said biomaterial implant is comprised of collagen mixed with α-gal nanoparticles or containing α-gal nanoparticles, cartilage fragments mixed with α-gal nanoparticles or bone fragments mixed with α-gal nanoparticles. In one embodiment, said biomaterial implant is dried or not dried and immersed in α-gal nanoparticles suspension for penetration of said α-gal nanoparticles into said biomaterial implant. In one embodiment, said biomaterial implant organ or tissue is perfused with α-gal nanoparticles suspension in order to introduce said α-gal nanoparticles into said biomaterial. In one embodiment, anti-Gal antibodies are bound to said α-gal nanoparticles. In one embodiment, said applying is under conditions such that complement activation within or adjacent to said biomaterial implant is enhanced. In one embodiment, said complement activation comprises production of complement fragments C5a, C4a and C3a. In one embodiment, said applying is under conditions for enhancing one or both of (a) monocyte and macrophage recruitment within or adjacent to said biomaterial implant, and (b) stem cell recruitment within or adjacent to said biomaterial implant. In one embodiment, said biomaterial is a synthetic biomaterial.

The invention also provides a method for inducing recruitment of macrophages into biomaterial implants for activation of said recruited macrophages to produce pro-healing cytokines and growth factors in a subject having endogenous anti-Gal antibody, comprising administering to said biomaterial composition that comprises a soluble molecules with one or more α-gal epitopes, wherein i) said soluble molecule α-gal carrying molecule comprises α-gal epitope having a terminal α-galactosyl and ii) said administering is under conditions for increasing the amount of macrophages in said biomaterial implant of said subject. In one embodiment, said terminal α-galactosyl is selected from the group consisting of Galα1-3Gal, Galα1-2Gal, Galα1-6Gal or any α-galactose sugar units capable of binding anti-Gal antibodies. In one embodiment, said α-gal epitope is free or part of a molecule selected from the group consisting of a natural or synthetic glycolipid, glycoprotein, and a glycopolymer. In one embodiment, said biomaterial is a natural tissue or organ selected from the group consisting of heart, urinary bladder, gall bladder, lung, trachea bronchi, bronchioles, alveoli, skeletal muscle, smooth muscle, connective tissue, ligament, cartilage, bone, nerve tissue, brain, spinal cord, liver, kidney, thyroid, parathyroid, pancreas, esophagus, stomach, small intestine, large intestine, eye, ear, and skin. In one embodiment, said biomaterial implant is comprised of collagen, cartilage fragments or bone fragments mixed with said soluble α-gal epitopes carrying molecule. In one embodiment, said biomaterial implant is dried or not dried and immersed in soluble α-gal epitopes carrying molecule suspension for penetration of said soluble α-gal epitopes carrying molecule into the biomaterial implant. In one embodiment, said biomaterial implant organ or tissue is perfused with soluble α-gal epitopes carrying molecules in order to introduce soluble α-gal epitopes carrying molecules into said biomaterial. In one embodiment, anti-Gal antibodies are bound to said soluble α-gal epitopes carrying molecule. In one embodiment, said applying is under conditions such that complement activation within or adjacent to said biomaterial implant is enhanced. In one embodiment, said complement activation comprises production of complement fragments C5a, C4a and C3a. In one embodiment, said applying is under conditions for enhancing one or both of (a) monocyte and macrophage recruitment within or adjacent to said biomaterial, and (b) stem cell recruitment within or adjacent to said biomaterial implant. In one embodiment, said biomaterial is a synthetic biomaterial.

The invention also provides a method for inducing recruitment of macrophages into injured internal tissues and for activation of said recruited macrophages to produce pro-healing cytokines and growth factors in a subject having endogenous anti-Gal antibody, comprising administering to said injured internal tissue composition that comprises α-gal nanoparticles, wherein. i) said α-gal nanoparticles comprises α-gal epitope having a terminal α-galactosyl and ii) said administering under conditions that increasing the amount of macrophages in said injured internal tissue of said subject. In one embodiment, said terminal α-galactosyl is selected from the group consisting of Galα1-3Gal, Galα1-2Gal, Galα1-6Gal or any α-galactose sugar units capable of binding anti-Gal antibodies. In one embodiment, said α-gal epitope is free or part of a molecule selected from the group consisting of a natural or synthetic glycolipid, glycoprotein, and a glycopolymer. In one embodiment, said α-gal nanoparticles are applied to injured internal tissues including: heart muscle, skeletal muscle, smooth muscle, connective tissue, ligament, bone, nerve tissue, brain, spinal cord, blood vessels, endocrine glands, exocrine glands, liver, kidney, gall bladder, thyroid, parathyroid, pancreas, esophagus, stomach, small intestine, large intestine, lung, trachea, bronchi, bronchioles, alveoli, eye, ear, ovary, testis, urinary bladder, skin. In one embodiment, said α-gal nanoparticles are applied to injured or severed fingers, toes, arms and feet. In one embodiment, said preparation is part of an injury care device selected from the group consisting of injections, adhesive bands, compression bandages, gels, semi-permeable films, plasma clots, fibrin clots, water, solutions, suspensions, emulsions, creams, ointments, aerosol sprays, collagen containing substances, stabilizers, sponges, drops, matrix-forming substances, foams or dried preparation. In one embodiment, said applying is under conditions such that complement activation within or adjacent to said injured tissue is enhanced. In one embodiment, said complement activation comprises production of complement cleavage chemotactic peptides including C5a, C4a and C3a. In one embodiment, said applying is under conditions for enhancing one or both of (a) monocyte and macrophage recruitment within or adjacent to said injured tissue, and (b) stem cell recruitment within or adjacent to said injured tissue. In one embodiment, said applying is under conditions such that injury healing and tissue repair and regeneration is induced or accelerated.

The invention further provides a method for inducing recruitment of macrophages into injured internal tissues and for activation of said recruited macrophages to produce pro-healing cytokines and growth factors in a subject having endogenous anti-Gal antibody, comprising administering to said injured internal tissue composition that comprises a soluble molecules with one or more α-gal epitopes, wherein i) said soluble molecule α-gal carrying molecule comprises α-gal epitope having a terminal α-galactosyl and ii) said administering under conditions that increasing the amount of macrophages in said injured internal tissue of said subject. In one embodiment, said terminal α-galactosyl is selected from the group consisting of Galα1-3Gal, Galα1-2Gal, Galα1-6Gal or any α-galactose sugar units capable of binding anti-Gal antibodies. In one embodiment, said α-gal epitope is free or part of a molecule selected from the group consisting of a natural or synthetic glycolipid, glycoprotein, and a glycopolymer. In one embodiment, said soluble α-gal epitope carrying molecules are applied to injured internal tissues including: heart muscle, skeletal muscle, smooth muscle, connective tissue, ligament, bone, nerve tissue, brain, spinal cord, liver, kidney, thyroid, parathyroid, pancreas, esophagus, stomach, small intestine, large intestine, lung, trachea, bronchioles, alveoli, eye, ear, glands, blood vessels, ovary, testis and skin. In one embodiment, said soluble α-gal epitope carrying molecules are applied to injured or severed fingers, toes, arms and feet. In one embodiment, said soluble α-gal epitope carrying molecules are part of a injury care device selected from the group consisting of injections, adhesive bands, compression bandages, gels, semi-permeable films, plasma clots, fibrin clots, water, solutions, suspensions, emulsions, creams, ointments, aerosol sprays, collagen containing substances, stabilizers, sponges, drops, matrix-forming substances, foams or dried preparation. In one embodiment, said applying is under conditions such that complement activation within or adjacent to said injured tissue is enhanced. In one embodiment, said complement activation comprises production of complement fragments C5a, C4a and C3a. In one embodiment, said applying is under conditions for enhancing one or both of (a) monocyte and macrophage recruitment within or adjacent to said injured tissue, and (b) stem cell recruitment within or adjacent to said injured tissue. In one embodiment, said applying is under conditions such that injury healing and tissue repair and regeneration is accelerated.

### BRIEF DESCRIPTION OF THE DRAWINGS

The following are illustrations of the present invention and are not intended to limit the scope of the invention in any manner.

FIG. 1A shows an interaction of an α-gal liposome with anti-Gal IgG and IgM antibodies. FIG. 1B illustrates an interaction between an anti-Gal coated (opsonized) α-gal liposome and a macrophage.

FIG. 2 shows the components of α-gal liposomes prepared from rabbit red blood cell (RBC) membranes. FIG. 2A depicts the separation of rabbit RBC glycolipids, phospholipids and cholesterol by thin layer chromatography (TLC), as demonstrated by nonspecific orcinol staining (left lane) and by immunostaining with an anti-Gal monoclonal antibody (mAb) designated Gal-13 (right lane) (Galili et al., J Immunol, 178: 4676, 2007). The smallest glycolipids having three carbohydrates (ceramide tri-hexoside [CTH]) lack α-gal epitopes and thus are not stained by the anti-Gal antibody. The number of carbohydrates in each α-gal glycolipid is indicated on the right. The smallest α-gal-containing glycolipid has five carbohydrates (ceramide pentahexoside [CPH]). FIG. 2B provides the structures of α-gal glycolipids having five, seven, 10, 15 and 20 carbohydrates, respectively.

FIGS. 3A and 3B show the binding of anti-Gal to α-gal liposomes either in an in vitro suspension or in a solid-phase antigen in an enzyme-linked immunosorbent assay (ELISA). FIG. 3A shows a graph of the binding of anti-Gal to α-gal liposomes in suspension as demonstrated by neutralization of anti-Gal in human serum. Serum was incubated with 10 mg/ml α-gal liposomes for 2 h at 37° C. Subsequently, the serum was placed in ELISA wells (in serial two-fold dilutions starting at a serum dilution of 1:10) coated with synthetic α-gal epitopes linked to bovine serum albumin (α-gal BSA) as the solid-phase antigen. The anti-Gal within the serum that was not neutralized by the α-gal liposomes bound to the α-gal BSA-coated wells. Binding of anti-Gal to α-gal BSA was determined by the subsequent binding of rabbit anti-human IgG coupled to horseradish peroxidase (HRP) and color development with O-phenylene diamine (OPD). Human serum incubated in the presence () or absence (◯) of α-gal liposomes is shown. FIG. 3B shows a graph of the binding of serum anti-Gal to α-gal liposomes as solid-phase antigen. α-Gal liposomes (100 μg/ml) in phosphate buffered saline (PBS) were dried in ELISA wells. After blocking with 1% BSA in PBS, the α-gal epitopes on α-gal liposomes in control wells were specifically removed from the glycolipids carbohydrate chains by incubation for 1 h at 37° C. with 10 units/ml recombinant α-galactosidase (α-galase). Anti-Gal readily binds to α-gal epitopes on the α-gal liposomes, and is evident even at a serum dilution of 1:320 (). Elimination of the terminal α-galactosyl unit by α-galactosidase results in complete elimination of the binding even at a serum dilution of 1:20 (0). Anti-Gal binding was evident in KO mouse serum dilution of 1:1280 (▪), whereas treatment of α-gal liposomes with α-galactosidase resulted in elimination of >99% of anti-Gal binding (□). Similarly, the anti-Gal monoclonal antibody (mAb) M86 bound effectively to the α-gal liposomes (♦). No significant binding was observed in wells treated with α-galactosidase (⋄). The lectin Bandeiraea simplicifolia IB4 (BS lectin with starting concentration of 10 μg/ml) that binds specifically to α-gal epitopes was observed to bind to α-gal liposomes (▴), but not to these liposomes after they were treated with α-galactosidase (Δ).

FIGS. 4A, 4B and 4C show the activation of human complement or rabbit complement by human anti-Gal binding to α-gal epitopes on α-gal liposomes. FIG. 4A shows a schematic for complement activity involving the lysis of the anti-Gal producing hybridoma cells M86. FIG. 4B provides a graph of showing the lysis of M86 cells by complement after incubation at 37° C. for 1 h. FIG. 4C provides a graph showing that interaction of human serum anti-Gal with α-gal liposomes results in complement consumption as measured by a loss of serum lytic activity. Human serum at a dilution of 1:10 was co-incubated with α-gal liposomes at various concentrations of the liposomes for 2 h at 37° C. This co-incubation results in a complete consumption of the complement due to complement activation and thus lose of cytolytic activity. FIG. 5 shows the migration of human monocytes and neutrophils, or of mouse macrophages in response to chemotactic gradients generated by complement activation following anti-Gal binding to α-gal liposomes. The analysis was performed in a Boyden chamber system. This system includes two chambers, with the lower chamber containing human serum mixed with α-gal liposomes and the upper chamber containing various peripheral blood mononuclear cells (PBMC) or polymorphonuclear cells (PMN). The two chambers are separated by a porous filter (e.g., 8 μm pores), which permits the migration of cells between the chambers. The size of the migration area is 18 mm². After 24 h at 37° C. the filters were washed and stained, and the number of cells migrating toward the lower chamber were counted. The study was performed with 1×10⁶ cells/ml in the upper chamber and serum diluted 1:5 and 1:10, mixed with 1 mg/ml of α-gal liposomes, in the lower chamber. Open columns indicate the number of migrating cells in the absence of serum; closed columns indicate the number of migrating cells with serum dilution 1:5; and gray columns indicate the number of migrating cells with serum dilution 1:10.

FIG. 6 depicts the in vivo induction of local inflammation by intradermal injection of α-gal liposomes in KO mice. The KO mice were immunized three times intraperitoneally with a homogenate of 50 mg pig kidney membranes to induce anti-Gal production. The KO mice were injected intradermally with 1 mg α-gal liposomes suspended in 0.1 ml saline, and euthanized at different time points post-injection. Skin specimens at the injection site were removed, sectioned, stained with hematoxyllin-eosin (H&E) and inspected microscopically. Some of the sections include the epidermis layer as point of reference. FIG. 6A shows untreated skin with the epidermis containing one or two layers of epithelial cells, and the dermis containing fibroblasts and fat cells (×100). FIG. 6B shows skin 12 hours post-injection (×100). FIG. 6C shows skin 12 h post-injection with the injection site at the center of the figure (×100). FIG. 6D shows skin 12 h post-injection (×400). Higher magnification of the infiltrating inflammatory cells indicates that the cells are neutrophils, based on the morphological characteristics of their nuclei. FIG. 6E shows skin 48 h post-injection (×400). The infiltrating inflammatory cells at this time point are mononuclear cells with characteristics of macrophages, as indicated by the kidney shape of many of these cells. FIG. 6F shows skin five days post-injection (×100). Most macrophages assume a round morphology because of internalization of numerous α-gal liposomes. The area in the center of the injection site is devoid of cells and is functioning as an α-gal liposome depot. FIG. 6G shows skin 14 days post-injection (×100) with macrophages still visible in area of the injection site. FIG. 6H shows skin 20 days post-injection (×100). The injection area contains many myofibroblasts differentiating into fibroblasts or muscle cells, and almost no macrophages are observed within the injected area.

FIG. 7 provides a graph depicting the lack of antibody response to injected α-gal liposomes. The antibody response was measured in an ELISA with 50 μl of α-gal liposomes at concentration of 100 μg/ml dried in each well (solid phase antigen). The dried α-gal liposomes were subsequently blocked with 1% BSA in PBS. Serum samples from two representative mice obtained before and 35 days post intradermal injection (◯, and □,▪), were tested for IgG binding to α-gal liposomes. No significant differences are observed in anti-α-gal liposomes IgG antibody activity in serum from mice obtained post α-gal liposome injection (closed symbols).

FIG. 8 provides exemplary data demonstrating in vivo recruitment of macrophages into polyvinyl alcohol (PVA) sponge containing α-gal liposomes. The sponge filled by soaking with α-gal liposome suspension (100 mg/ml) was implanted subcutaneously in α-1,3-galactosyltransferase knockout mice (KO mice) for 3 days, then removed. The infiltrating cells were obtained by repeated squeezing of the sponge in 1 ml PBS. The cells were stained with anti-CD11b antibody (Pharmingen, Inc,) that specifically binds to macrophages and allows for the identification of macrophages by flow cytometry (FACS) analysis. Solid line—isotype control of cells stained only with the secondary FITC coupled anti-rat IgG antibody. Broken line—cells stained with monoclonal rat anti-mouse CD11b Ab, then with secondary fluorescein coupled anti-rat IgG antibody. Note the shift of the whole cell population to the right, implying that all cells migrating into the PVA sponge containing α-gal liposomes, are macrophages. A representative mouse is shown.

FIG. 9 illustrates one embodiment of an interaction between anti-Gal and α-gal epitopes on α-gal glycolipids applied in the form of α-gal ointment. The α-gal ointment, comprised here of a mixture of α-gal liposomes (100 mg/ml) and petrolatum ointment (Vaseline), is applied topically on areas of damaged skin such as burns, in which serum proteins including anti-Gal and complement are released from damaged blood vessels. The α-gal epitopes (Galα1-3Galβ1-4GlcNAc-R) indicated on some of the chains in rectangles of broken lines) are present on all rabbit red cell glycolipids that carry 5 or more carbohydrate units (see FIG. 2). The present figure illustrates a representative α-gal glycolipid with 10 carbohydrate units. The fatty tail comprising the ceramide portion of the glycolipid enables mixing of the α-gal glycolipids within petrolatum (Vaseline) containing hydrocarbon chains of >25 carbons. α-Gal glycolipids within the ointment bind anti-Gal and thus, activate complement. The complement cleavage factors C5a and C3a recruit macrophages which mediate the accelerated natural process of wound healing.

FIG. 10 demonstrates one embodiment of an interaction between an anti-Gal antibody and α-gal glycolipids within α-gal ointment. Neutralization of monoclonal anti-Gal following mixing with α-gal ointment (◯), or Vaseline control lacking α-gal liposomes (). Anti-Gal activity was determined by subsequent binding to α-gal epitopes linked to BSA (α-gal BSA) as solid phase antigen in ELISA wells.

FIG. 11 provides exemplary data showing the effects of α-gal ointment on healing of burns induced by thermal injury to the skin. α1,3galactosyltransferase KO mice confirmed to produce anti-Gal in titers comparable to those in humans, were anesthetized and two burns were made on their backs by thermal injury with the heated bend end of a small metal spatula. One burn (left) was covered with Vaseline and the other (right) with α-gal ointment comprised of α-gal liposomes mixed with Vaseline. Subsequently, burns were covered with small round band-aids. After six days the band-aids were removed from the burns. As shown in (A), the burn treated with α-gal ointment healed significantly faster than that with Vaseline and its size was ˜50% of the Vaseline treated burn. Histological analysis of these burns demonstrated in the Vaseline treated burn (B) that the dermis was not covered by epidermis to replace the tissue damaged by the burn. The dark fragments on the skin are the damaged epidermis in the form of debris (crust) covering the wound and referred to as “eschar”. The α-gal ointment treated wound (C) also has eschar caused by the burn. However, the skin is covered by a new multilayered epidermis comprised of epithelial cells covered by the keratinous layer (stratum corneum, stained-pink). Data are of one of 4 mice with similar results.

FIG. 12 provides exemplary data showing rapid recruitment of macrophages into ischemic heart muscle injected with α-gal liposomes in mouse heart implanted subcutaneously (Hematoxyllin & Eosin staining). Hearts removed from KO mice were injected into the myocardium with 2 mg α-gal liposomes, or with saline. Subsequently, the hearts were implanted subcutaneously in KO mice producing anti-Gal.
FIG. 12A. Heart injected with saline obtained 2 week post implantation. Note the necrotic cardiomyocytes and the infiltrating neutrophils (×100).
FIG. 12B. Heart injected with α-gal liposomes obtained 2 week post implantation. Note the large number of infiltrating macrophages (×100).
FIG. 12C. As FIG. 12B, however the implanted heart was removed after 4 weeks. Note the border between the site of the α-gal liposomes injection (lower half) and the non-injected area which contains migrating macrophages (×200).
FIG. 12D. An area of the myocardium in α-gal liposomes injected hearts which lacks infiltrating cells, 2 weeks post implantation. Note that no nuclei are detected in the dead cardiomyocytes (×100).

FIG. 13 presents exemplary data showing rapid infiltration of macrophages into ischemic leg muscle treated with α-gal liposomes by intramuscular injection of 10 mg α-gal liposomes. The study was performed in anti-Gal producing KO mice in which the blood flow is blocked in the right hind leg by applying a rubber band tourniquet over the leg. The tourniquet is removed after 4 h to allow for reperfusion of the leg blood vessels. The histology studies are performed in the leg muscle Tibialis anterior.
FIG. 13A: Muscle fibers in an uninjured skeletal muscle comprising muscle cell syncitia (myotubes), formed by fusion of myoblasts, with nuclei in the periphery of the tubes.
FIG. 13B: Ischemia-induced myotube death after 96 hours, showing the resulting necrosis after sham injection with saline to serve as control to α-gal liposomes injection. Neutrophil infiltration of the necrotic tissue may be observed. Decreased myotube syncitia size is also observed wherein the nuclei of each myotube accumulate in a row. Subsequently, the dead myotubes are phagocytosed by debriding macrophages.
FIG. 13C: Ischemia-induced myotube death after 96 hours, showing improved structure after injection with 10 mg α-gal liposomes (H&E ×200).

FIG. 14 presents exemplary data showing the presence of cells with stem cell potential among the macrophages recruited into polyvinyl alcohol (PVA) sponge discs by anti-Gal/α-gal liposomes interaction. In view of the ability of stem cells to proliferate in vitro and form cell colonies, the cells migrating into implanted PVA sponge discs, due to chemotactic factors generated by anti-Gal/α-gal liposomes interaction, were tested for the ability to form colonies in vitro. The cells infiltrating into the implanted PVA sponge discs were retrieved and cultured in vitro on round cover slips in tissue culture wells for 5 days. Subsequently, the cover slips were stained with Wright staining.
FIG. 14A. Cells obtained from the subcutaneously implanted PVA sponge, 6 days post implantation. Most cells have the morphology of activated macrophages. The multiple vacuoles represent α-gal liposomes internalized by the macrophages. The bar represents 10 μm (×500).
FIGS. 14B and 14C: Infiltrating macrophage populations also include cells with stem cell characteristics that display an extensive ability to proliferate, i.e., to self renew, resulting in 200-500 cells per colony formed from one cell within a period of 5 days. Note the multiple mitotic cells in FIG. 14B. The frequency of these colony forming cells among cultured macrophages from PVA sponges is 3-5 cells/10⁵ macrophages. The colonies are representative of similar colonies from infiltrating macrophages in 5 mice.

FIG. 15. Exemplary binding of anti-Gal antibody coated α-gal liposomes to macrophages induces macrophage activation.
FIG. 15A: KO mouse peritoneal macrophages were incubated for 1 h at 4° C. with α-gal liposomes (1.0 mg/ml). The proportion of double stained cells representing α-gal liposomes bound to macrophages as measured by flow cytometry is indicated in the upper right corner. Data are representative for 3 independent studies.
FIG. 15B: KO mouse peritoneal macrophages were incubated for 1 h at 4° C. with anti-Gal antibody coated α-gal liposomes. The proportion of double stained cells representing α-gal liposomes bound to macrophages is indicated in the upper right corner. Data are representative for 3 independent studies.
FIG. 15C: Secretion of vascular endothelial growth factor (VEGF) by peritoneal macrophages co-cultured with anti-Gal antibody coated α-gal liposomes (closed columns); non-antibody coated α-gal liposomes (gray columns); or no liposomes (open columns). VEGF was quantified in culture media after 24 h or 48 h (Mean+SD [standard deviation] from the 4 mice/group). VEGF secretion by macrophages incubated with anti-Gal antibody coated α-gal liposomes is significantly higher than in the other two groups at each time point (p<0.05).

FIG. 16 presents exemplary data showing binding of anti-Gal IgG in KO mouse serum to α-gal liposomes (), or to KO pig liposomes, (◯) coating ELISA wells. Each curve represents serum from an individual mouse. The same sera were studied for IgG binding to both types of liposomes. KO pig liposomes are liposomes produced from α1,3GT knockout pig red blood cells (liposomes that lack α-gal epitopes).

FIG. 17 presents exemplary photomicrographs of in vivo recruitment of cells following subcutaneous injection of 10 mg α-gal liposomes in KO mouse skin. Specimens stained with hematoxylin and eosin (H&E). For the purpose of orientation, the epidermis is shown in the upper areas of FIGS. 17B, 17C, 17G, 17H and 17I. Each figure is representative of 5 mice/group.
FIG. 17A: 12 h post injection. Empty areas represent injected liposomes that were dissolved during the staining process (×100). Scale bar=100 μm.
FIG. 17B: 24 h post injection (×100).
FIG. 17C: 24 h post injection of α-gal liposomes and 20 μg cobra venom factor (CVF) (×100).
FIG. 17D: The inset in FIG. 17A, showing recruited neutrophils (×200).
FIG. 17E: The inset in FIG. 17B indicating that most (>85%) of the cells have morphology of macrophages (×200).
FIG. 17F: 24 h post injection of 10 mg KO pig liposomes (i.e. liposomes that lack α-gal glycolipids).
FIG. 17G: 6 days post injection of α-gal liposomes (×100).
FIG. 17H: 14 days post injection of α-gal liposomes (×100).
FIG. 17I: 28 days post injection of α-gal liposomes (×100).
FIG. 17J: The inset in FIG. 17G, indicating that the mass of cells by the injection site is comprised of large macrophages containing multiple vacuoles that represent internalized phagocytozed α-gal liposomes (×200). Scale bar=50 μm
FIG. 17K: 96 h post injection. The section is immunostained with HRP-anti-4/F80 which stains macrophages in brown (×400), and counterstained with H&E.
FIG. 17L: Morphology of individual recruited macrophages, 6 days post injection. The multiple vacuoles represent the anti-Gal antibody coated α-gal liposomes internalized by the macrophages (×1000). Scale bar=10 μm.

FIG. 18 presents exemplary data showing the recruitment of neutrophils (grey columns) and macrophages (closed columns) by 10 mg liposomes injected subcutaneously in 0.1 ml suspension into KO mice. α-gal lipo CVF-α-gal liposomes were co-injected with 20 μg cobra venom factor (inhibits complement activity). “KO pig lipo” are liposomes produced from α1,3GT knockout pig red blood cells (liposomes that lack α-gal epitopes). Number of infiltrating cells was determined in histological sections by counting cells within a rectangular area marked in a microscope lens at magnification of ×400, corresponding to 100×200 μm area. The differences in number of macrophages in day 1 (24 h) to day 14 are not significant. Mean+SD from 5 mice/group.

FIG. 19 presents exemplary photomicrographs showing redness adjacent to subcutaneous injection sites of α-gal liposomes (left column), or KO pig liposome (right column) in KO mice, viewed 48 h post injection (×4). The injected liposomes appear as white areas viewed from the basal side of the skin. Note the redness caused by vasodilation and/or angiogenesis near the α-gal liposome injected areas, but not by the KO pig liposome injected areas.

FIG. 20 presents exemplary data showing the activation of cytokine gene expression in macrophages as measured by quantitative real time polymerase chain reaction (q-RT-PCR) as fold-changes in expression of the various cytokine genes. RNA extracts were taken from 5 KO mice injected in the skin with α-gal liposomes and harvested after 48 h, compared to saline injected skin as control. The bottom right figure represents the Mean+SD, except for Il1a where the mean−SD is presented.

FIG. 21 presents exemplary data showing the activation of cytokine gene expression in macrophages as measured by q-RT-PCR as fold changes in cytokine gene expression. Peritoneal macrophages were harvested 24 h post i.p. injection of 30 mg α-gal liposomes and compared to peritoneal macrophages from saline injected KO mice as a control. Each color represents a different mouse.

FIG. 22 presents exemplary wound healing data taken at different time points after epidermal excisional wound formation and topical application of a dressing covered with either: i) 10 mg α-gal liposomes (hatched columns); ii) 10 mg KO pig liposomes lacking α-gal epitopes (grey columns); or iii) saline (open columns); iiii) 10 mg α-gal nonoparticles (closed columns);. Extent of wound healing is described as % of the wound area covered with regenerating epidermis. On day 3, n=11 for all groups. On day 6, n=20 for mice with wounds treated with α-gal liposomes or with saline and n=11 for mice treated with α-gal nanoparticles and those treated with KO pig liposomes. On day 9, n=8 for all groups, whereas n=11 for all groups on day 12. Data presented as Mean+SD (p<0.05).

FIG. 23 presents exemplary micrographs showing healing of representative excisional wounds treated with spot bandages covered with 10 mg α-gal liposomes, or with saline (hematoxylin and eosin [H&E]). Specimens are representative of 7 mice treated with α-gal liposome (complete wound closure) and of 20 mice treated with saline.
FIG. 23A: Control wound treated with saline dressing for 3 days. Panniculus carnosus is exposed where epidermis and dermis were removed. No significant cell infiltration is observed. (×100)
FIG. 23B: Wound treated for 3 days with α-gal liposomes. Note the multilayered proliferating epidermis at the periphery of the wound and infiltration of macrophages in the regenerating dermis. The platelet plug is present above the healing wound. Arrow marks the wound edge. ×200
FIG. 23C: Day 6 saline treated wound (center of the wound). Regenerating thin dermis over Panniculus carnosus is filled with macrophages. No regenerating epidermis is observed. ×200
FIG. 23D: Day 6 wound treated with α-gal liposomes (center of the wound). Note that multi-layered regenerating epidermis covers the entire area of the wound and many macrophages infiltrate the dermis. ×200
FIG. 23E: Day 6 saline treated wound (periphery of the wound). The regenerating epidermis in the lower left area does not cover the entire wound. The dermis is filled with macrophages. ×200
FIG. 23F: Day 6 wound treated with α-gal liposomes (periphery of the wound). The uninjured skin is observed in the right area. The dermis of the wound is filled with macrophages. Arrow marks the wound bed. Pink stratum corneum is observed over the regenerating epidermis. ×200; Scale bar=50 μm.

FIG. 24 presents exemplary micrographs showing the gross appearance of day 6 excisional wounds treated with dressing (spot bandages) covered with: saline (First Column: 5 wounds); KO pig liposomes lacking α-gal epitopes (Second Column: 5 wounds); or α-gal liposomes (Third and Fourth Columns: 10 wounds). The extent of wound healing is evaluated by the estimated proportion (%) of the wound covered with regenerating epidermis (i.e., % of healing) is indicated within each figure. ×2-4.

FIG. 25 presents exemplary photomicrographs showing Trichrome staining of regenerating dermis in the skin wounds treated with dressing (spot bandages) covered with saline (A, C and E) or with α-gal liposomes (B, D and F), as detailed in FIGS. 22 and 23. In this staining, collagen is blue and the various cells are purple. The border of the wound bed between uninjured and regenerating tissues is marked with arrows in 25B, 25E and 25F. Magnification in 25A is ×100, in 25B-F ×200. Scale bar in 25F is 50 μm. Specimens are representative of 7 mice treated with α-gal liposome (complete wound closure) and of 20 mice treated with saline.

FIG. 26 presents exemplary photomicrographs showing that α-gal liposome treatment decreases scar formation. H&E—(A, C, E and G) and Trichrome staining—(collagen stained blue in B, D, F and H) of wounds treated for 28 days with saline or with α-gal liposomes, as indicated. Saline treated wounds develop a scar characterized by dense connective tissue due to multiple fibroblasts (the two specimens in A, B and C, D) and no skin appendages such as hair and sebaceous glands. In contrast, α-gal liposomes treated wounds (the two specimens in E, F and G, H) display restoration of normal skin histology, including thin epidermis, loose connective tissue in the dermis and appearance of skin appendages including hair and sebaceous glands, as well as fat cells in the hypodermis. Scale bar in C is 100 μm (×100). Specimens are representative of 5 mice/group.

FIG. 27 presents exemplary photomicrographs of the histological (H&E) characteristics of skin burns covered with spot bandages that were coated with 10 mg α-gal liposomes, or with control bandages coated with saline. The burns were examined at various time points as pairs obtained from the same mouse and are representative of five mice at each time point. (A) is an exception presenting normal noninjured KO mouse skin (×200).

FIG. 28 presents exemplary data showing a representative analysis of an immune response to α-gal liposomes. Anti-liposomes IgG antibodies were measured by ELISA in wells coated with to α-gal liposomes as solid phase antigen. KO mice immunized with pig kidney membranes (PKM) homogenate served as positive control.

FIG. 28A: Naive KO mice receiving two i.p. injections of 10 mg α-gal liposomes at 1 week intervals. (Δ)-sera from mice injected with α-gal liposomes; (•)-sera from mice injected with PKM; (∘)-sera from mice injected with PKM, were preincubated for 30 min with 1 mg/ml α-gal BSA.
FIG. 28B: Naïve KO mice receiving topical application of α-gal liposomes onto burns for a period of 2 weeks. (Δ)-sera from mice with burns treated topically with α-gal liposomes. Curves of (Δ) and (∘) represent no IgG binding. Data from three mice in each group.

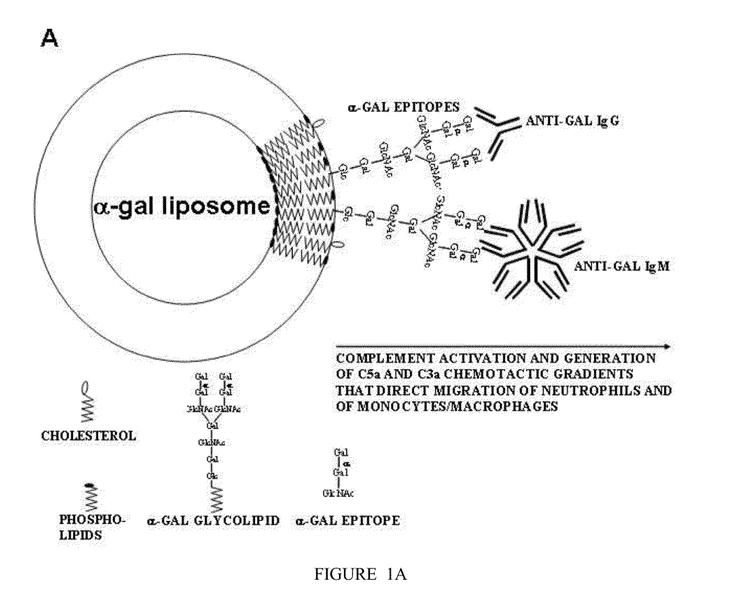

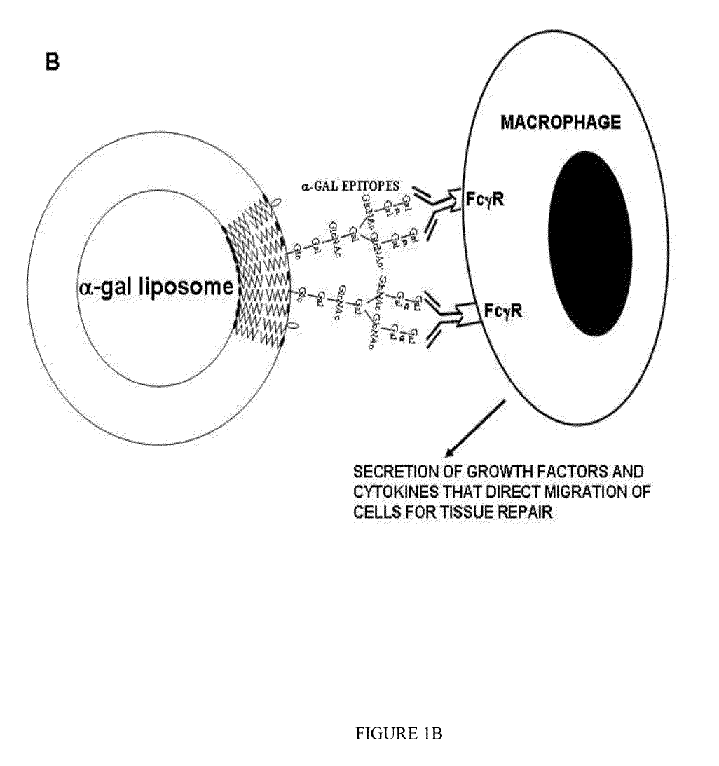

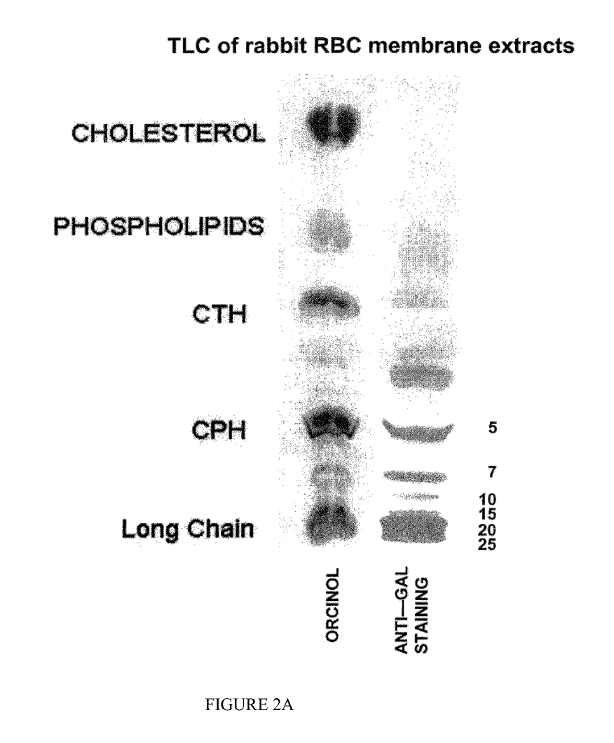

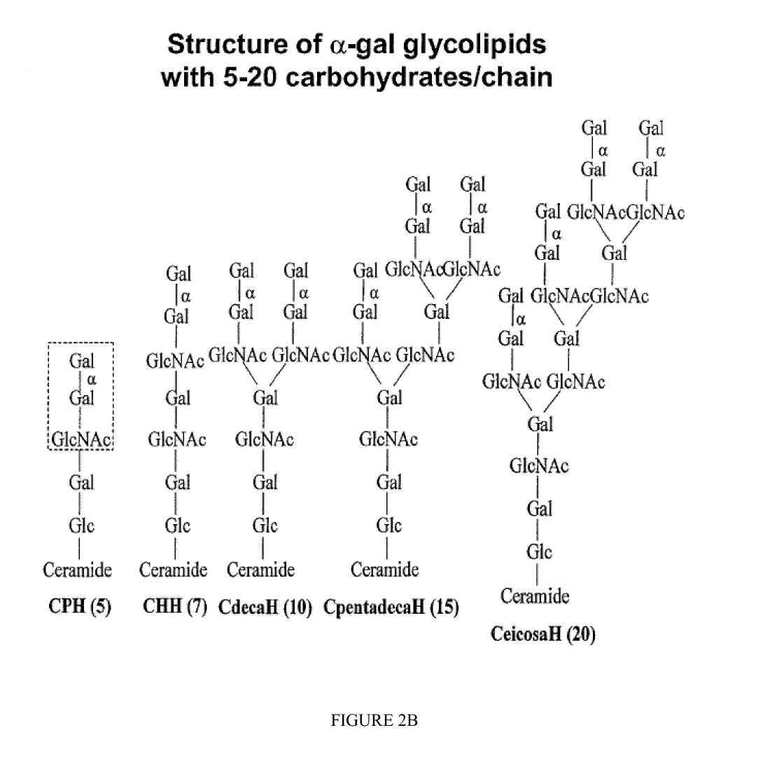

FIG. 29 presents a schematic illustration of an α-gal nanoparticle with α-gal glycolipids containing 10 carbohydrates in two branches, each capped with an α-gal epitope (marked by a dashed line rectangle) as representative α-gal glycolipids. The α-gal nanoparticles are α-gal liposomes with the molecular composition identical to that in FIG. 2. The α-gal nanoparticles are generated by decreasing the size of the α-gal liposomes described in FIGS. 1 and 2 to submicroscopic size using a sonication probe. When α-gal nanoparticles are applied into an injured tissue, or when a biomaterial such as, but not limited to, a natural tissue or organ, containing α-gal nanoparticles are implanted in humans, binding of the natural anti-Gal antibody to the α-gal epitopes on the nanoparticles results in activation of the complement system. The pro-healing cytokines/growth factors which also recruit stem cells produced chemotactic complement cleavage peptides induce rapid recruitment of macrophages. Anti-Gal coating α-gal nanoparticles further interacts via its Fc “tail” with Fcγ receptors (FcγR) on macrophages. This interaction activates macrophages to produce and secrete a variety of. The components of the α-gal nanoparticles are illustrated at the bottom of the figure.

FIG. 30 describes the binding of anti-Gal coated α-gal nanoparticles to adherent α1,3galactosyltransferase knockout pig (KO pig) macrophages, as evaluated by scanning electron microscopy. A-C. The α-gal nanoparticles coated with natural KO pig anti-Gal antibody were incubated with adherent KO pig macrophages for 2 h at room temp. The macrophages were then extensively washed to remove nonadherent nanoparticles and subjected to scanning electron microscopy processing and analysis. The surface of the representative macrophage is covered with α-gal nanoparticles as a result of Fc/FcγR interaction as illustrated in FIG. 29. The inset in (A) is enlarged in (B). In (A) and (B) the size of the α-gal nanoparticles is ˜100-300 nm, in (C) the size is 10-30 nm. D. A macrophage incubated with α-gal nanoparticles that were not coated with anti-Gal antibody. No α-gal nanoparticles bind to the macrophage in the absence of the coating anti-Gal antibody. Each panel is a representative of 10 macrophages with similar morphology.

FIG. 31 demonstrates the rapid recruitment of macrophages by α-gal nanoparticles injected into the skin. α1,3galactosyltransferase knockout (KO) mouse skin was injected with 1 mg of α-gal nanoparticles in 0.1 ml saline. (A) The injection site after 24 h demonstrates recruitment of macrophages around the injection site. This injection site is indicated as the empty area since the α-gal nanoparticles are dissolved and washed away by the alcohol use in the hematoxylin & eosin (H&E) staining process (×100). (B) The injection site after 48 h demonstrating increased recruitment of macrophages (×100). (C) The injection area after 6 days showing multiple activated macrophages bordering each other and displaying ample cytoplasm due to the activation process (×200). (D) Control injection sites injected only with saline (the vehicle for α-gal nanoparticles) and inspected after 48 h. The injection sites display no recruitment of macrophages (×100). Representative specimens of 5 mice with similar results.

FIG. 32. Demonstrates the pluripotent/pluripotential characteristics of stem cells recruited by α-gal nanoparticles into PVA sponge discs. PVA sponge discs containing 0.15 ml of a suspension of 1 mg/ml α-gal nanoparticles and implanted for five weeks under the skin of anti-Gal producing KO mice. The retrieved PVA discs were sectioned and stained with hematoxylin-eosin (H&E) (A) or with trichrome (for staining collagen blue) (B). (A) Generation of nerve fibers comprised of multiple axons (represented by the three organized horizontal bundles) indicating stem cell differentiation into nerve tissue and of blood vessels (lower left corner and upper left corner under the letter A) indicating angiogenesis induced following the recruitment of macrophages by α-gal nanoparticles within the PVA implant (×200). (B) Generation of myotubes (four horizontal red structures, striation in the two upper myotubes can be observed upon magnification of the picture) and of connective tissue with secreted collagen stained blue by the trichrome staining (×200). The PVA sponge material is stained as grey in (A) and grey blue reticular material in (B). The multiple types of cells in subcutaneously implanted PVA sponge discs containing α-gal nanoparticles, including nerve cells, muscle cells and fibroblasts imply that the stem cells recruited by the activated macrophages are pluripotent/pluripotential. The blood vessels observed in the PVA sponge discs further imply that the activated macrophages induce angiogenesis within the implants. Implants lacking α-gal nanoparticles display few cells which primarily are fibroblasts and adipocytes as in FIG. 35E below.

FIG. 33 illustrates the recruitment of stem cells by activated macrophages as indicated by the differentiation of the recruited stem cells into fibrochondrocytes according to cues provided by meniscus cartilage extracellular matrix (ECM). Polyvinyl alcohol (PVA) sponge discs containing α-gal nanoparticles (10 mg/ml) and pig meniscus cartilage fragments homogenate (10 mg/ml) were implanted subcutaneously for five weeks in KO mice The cartilage was depleted of α-gal epitopes prior to homogenization by 20 h incubation at 24° C. with 100 Units/ml recombinant α-galactosidase, followed by repeated washes for removal of the enzyme, as previously described (Stone et al. Transplantation 65:1577, 1998). The retrieved PVA sponge discs were fixed, sectioned and stained with H&E or with trichrome (for staining collagen in blue). A. A section of a PVA sponge disc with areas of fibrocartilage growth (marked by the rectangles) (H&E ×20). The sponge PVA material is stained dark purple. B. Generation of fibrocartilage tissue within the PVA sponge disc. The fibrocartilage is stained red and the PVA sponge dark purple. The few fibrochondrocytes which secreted the cartilage matrix are identified by the dark purple stained nuclei of these cells (H&E, ×200). C. Fibrocartilage formed within the PVA sponge disc stained with trichrome. The collagen of the fibrocartilage matrix is stained blue and the sponge is stained grey blue (×200). D. Magnification of the area within the rectangle in (C). The fibrous organization of the fibrocartilage characteristic to meniscus cartilage is readily seen. The relatively few fibrochondrocytes are indicated by their nuclei stained deep purple (×400). Most of the area between cells is filled with de novo secreted collagen fibers. E. PVA sponge disc containing meniscus homogenate but lacking α-gal nanoparticles. The sponge (stained purple grey) contains clusters of fat cells and some connective tissue and no fibrocartilage (H&E, ×200). F. Pig meniscus cartilage (H&E, ×400). The histology of this unprocessed tissue is similar to that in FIGS. 33B, 33C and 33D with the exception that in the original meniscus cartilage tissue the cells and fibrocartilage matrix have a parallel organization whereas in the sponge disc they are organized in different directions because of the structure of the PVA sponge. The histological analysis indicates that the combination of fragmented pig cartilage and α-gal nanoparticles induces the generation of cartilage within the sponge, whereas in the absence of α-gal nanoparticle, no significant cartilage formation is observed. These observations confirm the effects of the α-gal nanoparticles which interact within the PVA sponge spaces with the anti-Gal antibody and generate complement cleavage chemotactic factors that recruit macrophages. These macrophages are activated as a result of binding via their Fcγ receptors the Fc portion of anti-Gal bound to the α-gal nanoparticles. The activated macrophages produce and secrete cytokines/growth factors that recruit stem cells and induce angiogenesis within the implanted sponges. The recruited stem cells are instructed by the ECM of the meniscus cartilage fragments to differentiate into fibrochondroblasts that secrete fibrocartilage, similar to that in the meniscus cartilage. Representative sponge discs from 5 mice having the same treatment.

FIG. 34 demonstrates the recruitment of macrophages into ischemic heart tissue by α-gal nanoparticles. The myocardium of KO mouse harvested hearts was injected with 1 mg α-gal nanoparticles in 0.1 ml saline prior to subcutaneous implantation into KO mice producing the anti-Gal antibody. The implants were not connected to the blood circulation and were harvested 4 weeks, and subjected to H&E staining. A. The border of the α-gal nanoparticles injected myocardium. The injected myocardium is filled with recruited macrophages which infiltrate into the noninjected area structure of the ischemic myocardium near the injected area is conserved (×100). B. A region of the implanted heart far from the α-gal nanoparticles injected site. Macrophages recruited by α-gal nanoparticles infiltrated this region and the structure of the mycocardium is maintained despite the death of the ischemic cardiomyocytes which lack nuclei (×100). A representative of 5 KO mice with similar results.

FIG. 35 describes the recruitment of macrophages into KO pig heart by endomyocardial injection of α-gal nanoparticles (100 mg/ml) by using an injection catheter that was navigated into the left ventricle and injected into the myocardium. The injection was in a area near the endocardium of the healthy pig heart. (A) Heart of a pig euthanized after 5 days. Note the multiple macrophages migrating within into the injection area which is identified by the empty area in the damage myocardium. (B) Heart of a pig euthanized after 7 days. Note “raws” of recruited macrophages (cells with large oval nucleus) migrating away from the injection area between the cardiomyocytes (×100). and the myocardium sectioned and processed for histological analysis. (A) a and to a lesser extent along the route of injection (empty areas) (B) (×100 H&E).

FIG. 36 describes the recruitment of macrophages into a plasma clot containing α-gal nanoparticles as an example of a semi-solid filler or gel applied to internal and external injuries. Plasma from human blood was mixed with α-gal nanoparticles (10 mg/ml) and was induced to form a clot by addition of 10 mM calcium chloride. These clots representing a gel containing α-gal nanoparticles were placed on skin wounds of anti-Gal producing α1,3galactosyltransferase (α1,3GT) knockout mice (KO mice). A. A distinct infiltration of macrophages is observed within 3 days after placing the clot on the wound. B. Six days after placing the plasma clot containing α-gal nanoparticles on the wound, the clot is filled with macrophages that were recruited into it as a result of anti-Gal/α-gal nanoparticle interaction. It is contemplated that the complete regeneration of the epidermis is associated with the activity of cytokines/growth factors secreted from the macrophages recruited into the clot and activated by the anti-Gal coated α-gal nanoparticles (as illustrated in FIGS. 1 and 29). The broken line marks the approximate edge of the clot.

FIG. 37 shows the morphology of wounds on α1,3GT knockout pigs (KO pigs) treated for 13 days by topical application of 100 mg α-gal nanoparticles. The original size of the excisional wounds was 20×20 mm and 3 mm deep. The border of the wounds was marked by 8 tattooed dots. Control wounds treated with saline display partial regeneration of the epidermis as a result of physiologic healing. However, wounds treated with 100 mg α-gal nanoparticles are almost completely or completely covered with regenerating epidermis. The shape of stretched tattooed dots indicates the extent of wound contraction and implies that there are no significant differences wound contractions between saline treated and α-gal nanoparticles treated wounds.

FIG. 38 describes the induction of angiogenesis by macrophages recruited into representative wounds of KO pig 7 days and 13 days following treatment with 100 mg α-gal nanoparticles or with saline (as in FIG. 37 above). (A) The center of wound (not covered by regenerating epidermis) treated with α-gal nanoparticles on day 7. (B) Center of wound treated with saline on day 7. (C) Center of wound treated with α-gal nanoparticles on day 13. (D) Center of wound treated with saline on day 13. (E) Wound area under the leading edge of regenerating epidermis in α-gal nanoparticles treated wounds on day 13. (F) Area under the leading edge of regenerating epidermis in saline treated wounds on day 13. Note that both in the center of the wound (A and C) and under the leading edge of the epidermis (E) there are many more macrophages in wound treated with α-gal nanoparticles than those treated with saline (B, D and F respectively). In addition, there are many more blood vessels in the center of the wound (C) and under the leading edge of the regenerating epidermis (E) in α-gal nanoparticles treated wounds than in wounds treated with saline (D and F, respectively). These findings indicate that α-gal nanoparticles recruit macrophages in KO pigs similar to the recruitment in KO mice describes in FIG. 31 above. Furthermore, the activation of the macrophages following their binding the Fc portion of anti-Gal coating α-gal nanoparticles results in the secretion of angiogenic factors (e.g. VGEF) that induced a much higher level of angiogenesis than in saline treated pig wounds. Representative wounds from 2 KO pigs euthanized on day 7 and 6 KO pigs euthanized on day 13 after wounding and treatment with α-gal nanoparticles or saline (H&E ×200).

### DEFINITIONS

To facilitate an understanding of the present invention, a number of terms and phrases are defined below:

The term “α-gal epitope” as used herein, refers to any molecule, or part of a molecule, with a terminal structure comprising Galα1-3Galβ1-4GlcNAc-R, Galα1-3Galβ1-3GlcNAc-R, or any carbohydrate chain with terminal Galα1-3Gal at the non-reducing end, or any molecule with terminal α-galactosyl unit capable of binding the anti-Gal antibody.

The term “glycolipid” as used herein, refers to any molecule with at least one carbohydrate chain linked to a ceramide, or a fatty acid chain, or any other lipid. Alternatively, a glycolipid maybe referred to as a glycosphingolipid.

The term “α-gal glycolipid” as used herein, refers to any glycolipid that has at least one α-gal epitope on its non-reducing end of the carbohydrate chain.

The term “α-gal epitope mimicking peptides” as used herein, refers to any peptide that is capable of binding the anti-Gal antibody.

The term “α-gal liposomes” as used herein, refers to any liposomes that have α-gal epitopes and are capable of binding the anti-Gal antibody. In one embodiment, “α-gal liposomes” comprise natural and/or synthetic phospholipids, and/or other lipids that are comprised of hydrocarbon base, and/or any other base which contains α-gal epitopes or of α-gal epitopes in α-gal glycolipids, α-gal proteins, α-gal proteoglycans, and/or α-gal polymers and which may or may not be comprised also of cholesterol. The α-gal liposomes may be of any size including, but not limited to, the range of approximately 0.1-1000 micrometer (μm). In a particular embodiment, the α-gal liposomes are referred to as “α-gal nanoparticle.

The term “nanoparticle” as exemplified by “α-gal nanoparticle” refers to a liposome having size range of from 0.0001 μm to 1000 μm. In one embodiment, the nanoparticle is from 0.1 μm to 1000 μm. In another embodiment, the nanoparticle is from 0.0001 μm to 0.5 μm. In one embodiment, the “α-gal nanoparticles” comprise natural and/or synthetic materials that present α-gal epitopes. α-gal epitopes may be part of α-gal glycolipids, α-gal glycoproteins, α-gal proteoglycans, synthetic α-gal comprised molecules or α-gal polymers. In one embodiment, the α-gal nanoparticles comprise phospholipids, α-gal glycolipids and cholesterol and are produced from chloroform:methanol extracts of rabbit red cell membranes. In a preferred embodiment the size range of α-gal nanoparticles is 0.0001-0.5 μm.

The term “α-gal ointment” as used herein, refers to any ointment of hydrocarbon base or any other base that contains α-gal epitopes in a free form or α-gal epitopes in α-gal glycolipids, α-gal proteins, or α-gal polymers.

The terms “pro-healing cytokines and growth factors” and “pro-healing cytokines/growth factors” refer to cytokines and growth factors that are secreted by activated macrophages and which mediate healing, repair and regeneration of various external and internal injuries. Some of these cytokines and growth factors also recruit stem cells into and around the areas in which they are secreted from macrophages.

As used herein, the term “purified” refers to molecules (polynucleotides, or polypeptides, or glycolipids) that are removed from their natural environment, isolated or separated. “Substantially purified” molecules are at least 50% free, preferably at least 75% free, more preferably at least 90% and most preferably at least 95% free from other components with which they are naturally associated.

The terms “α1,3-galactosyltransferase,” “α-1,3-galactosyltransferase,” “α1,3GT,” “glycoprotein α-galactosyltransferase 1” and “GGTA1,” as used herein refer to any enzyme capable of synthesizing α-gal epitopes. The enzyme is expressed in most mammals (e.g., nonprimate) but not in humans, apes and Old World monkeys. The carbohydrate structure produced by the enzyme is immunogenic in man and most healthy people have high titer natural anti α-gal antibodies, also referred to as “anti-Gal” antibodies. In some embodiments, the term “α1,3GT” refers to a common marmoset gene (e.g., Callithrix jacchus—GENBANK Accession No. 571333) and its gene product, as well as its functional mammalian counterparts (e.g., other New World monkeys, prosimians and non-primate mammals, but not Old World monkeys, apes and humans). The term “α1,3GT” is in no way limited to a particular mammal, for example, the term may include mouse α1,3GT (e.g., Mus musculus—nucleotides 445 to 1560 of GENBANK Accession No. NM_{—}010283), bovine α1,3GT (e.g., Bos taurus—GENBANK Accession No. NM_{—}177511), feline α1,3GT (e.g., Felis catus—GENBANK Accession No. NM_{—}001009308), ovine α1,3GT (e.g., Ovis aries—GENBANK Accession No. NM_{—}001009764), rat α1,3GT (e.g., Rattus norvegicus—GENBANK Accession No. NM_{—}145674) and porcine α1,3GT (e.g., Sus scrofa—GENBANK Accession No. NM_{—}213810). Some embodiments of the present invention comprise a functional variant of a mammalian α1,3GT, which differs from the wild type mammalian α1,3GT sequences in, for example, fewer than 1-5% of the residues. α1,3GT variants include but are in no way limited to naturally occurring, functional mammalian α1,3GT variants, as well as non-naturally occurring variants generated by recombinant or other means (e.g., 1, 2, 3, 4 or 5 amino acid substitutions, deletions, or additions, preferably corresponding to a residue from a functional mammalian α1,3GT homolog) are contemplated to find use in the compositions and methods of the present invention. In other embodiments, truncated forms of a mammalian α1,3GT, which retain catalytic activity, are employed (e.g., GGTA1 lacking 90 amino acid N-terminal stem region).

The term “KO mouse” as used herein refers to any mouse in which the α1,3GT gene was knocked out, i.e. disrupted, to prevent synthesis of self α-gal epitopes, thereby enabling production of anti-Gal antibodies by the mouse.

The term “KO pig” as used herein refers to any pig in which the α1,3GT gene was knocked out, i.e. disrupted, to prevent synthesis of self α-gal epitopes.

The term “anti-Gal binding epitope”, as used herein, refers to any molecule or part of molecule that is capable of binding in vivo the natural anti-Gal antibody.

The term “isolated” as used herein, refers to any composition or mixture that has undergone a laboratory purification procedure including, but not limited to, extraction, centrifugation and chromatographic separation (e.g., thin layer chromatography or high performance liquid chromatography). Usually such purification procedures provide an isolated composition or mixture based upon physical, chemical, or electrical potential properties. Depending upon the choice of procedure an isolated composition or mixture may contain other compositions, compounds or mixtures having similar chemical properties.

The term “control” refers to subjects or samples which provide a basis for comparison for experimental subjects or samples. For instance, the use of control subjects or samples permits determinations to be made regarding the efficacy of experimental procedures. In some embodiments, the term “control subject” refers to animals, which receive a mock treatment (e.g., saline).

The term “diabetic” as used here refers to organisms which have a disorder characterized by the insufficient production or utilization of insulin. Insulin is a pancreatic hormone that is needed to convert glucose for cellular metabolism and energy production. In preferred embodiments of the present invention, the term “diabetic patient” refers to patients suffering from diabetes mellitus. The term “diabetic” encompasses both patients with type I diabetes (juvenile onset) and patients with type II diabetes (adult onset). “Type I diabetes” also referred to as “insulin-dependent diabetes” is a form of diabetes mellitus that usually develops during childhood or adolescence and is characterized by a severe deficiency in insulin secretion resulting from atrophy of the islets of Langerhans and causing hyperglycemia and a marked tendency towards ketoacidosis. “Type II diabetes” also referred to as “non-insulin-dependent diabetes” is a form of diabetes mellitus that develops especially in adults (most often in obese individuals) and that is characterized by hyperglycemia resulting from both insulin-resistance and an inability to produce more insulin.

The term “aged” as used herein refer to older human subjects (e.g., middle age and above of 50 years and older, senior citizen and above of 65 years and older, or elderly and above of 80 years and older, etc.). The term “aged” also encompass older nonhuman mammalian subjects at similar stages in their life cycles (e.g., 8-12 years and older for cats and large dogs, 10-15 years and older for small and medium sized dogs, 15-18 months and older for mice, etc.)

The terms “patient” and “subject” refer to a mammal or an animal that is a candidate for receiving medical treatment.

As used herein, the term “wound” refers to a disruption of the normal continuity of structures caused by a physical (e.g., mechanical) force, a biological (e.g., thermic or actinic force, or a chemical means. In particular, the term “wound” encompasses wounds of the skin. The term “wound” also encompasses contused wounds, as well as incised, stab, lacerated, open, penetrating, puncture, abrasions, grazes, burns, frostbites, corrosions, wounds caused by ripping, scratching, pressure, and biting, and other types of wounds. In particular, the term encompasses ulcerations (i.e., ulcers), preferably ulcers of the skin.

As used herein, the term “injured tissue” refers to a disruption of the normal continuity of structures caused by a physical (e.g., mechanical) force such as in incisions caused by surgery, a biological (e.g., thermic or actinic force), or a chemical means. In particular, the term “tissue injury” encompasses ischemia in various tissues, stroke in the brain, disappearance of secretory cells in endocrine glands, ischemia of muscles, severed neural exons and any disruption of normal structure and function of tissues. The term “injured tissue” also encompasses contused tissues, as well as incised, stab, lacerated, open, penetrating, puncture, injuries caused by ripping, scratching, pressure, and biting. The term further encompasses ulcerations (i.e., ulcers), preferably ulcers of the gastrointestinal track.

As used herein, the term “damaged tissue” refers to a destruction of a tissue normal structure and damage to the normal tissue function because of disease or because of exposure to damaging agents. A non-limiting example is the prolonged exposure to smoke due to cigarette smoking which results in damaged tissue in the lungs.

As used herein, the term “tissue repair and regeneration” refers to a regenerative process with the induction of an exact temporal and spatial healing program that encompasses but is not limited to the processes of granulation, neovascularization, fibroblast, endothelial and epithelial cell migration, extracellular matrix deposition, re-epithelialization, reappearance of the cells characteristic to the treated tissue prior to the injury and remodelling of the tissue.

As used herein, the term “wound healing” refers to a regenerative process with the induction of an exact temporal and spatial healing program comprising wound closure and the processes involved in wound closure. The term “wound healing” encompasses but is not limited to the processes of granulation, neovascularization, fibroblast, endothelial and epithelial cell migration, extracellular matrix deposition, re-epithelialization, and remodeling.

The term “wound closure” refers to the healing of a wound wherein sides of the wound are rejoined to form a continuous barrier (e.g., intact skin).

The term “granulation” refers to the process whereby small, red, grain-like prominences form on a raw surface (that of wounds or ulcers) as healing agents. In one embodiment, the term “granulation” refers to the process whereby small, red, grain-like prominences form on a raw surface (that of injured tissues or ulcers) as healing agents.

The term “neovascularization” refers to the new growth of blood vessels with the result that the oxygen and nutrient supply is improved. Similarly, the term “angiogenesis” refers to the vascularization process involving the development of new capillary blood vessels.

The term “cell migration” refers to the movement of cells (e.g., fibroblast, endothelial, epithelial, stem cells, etc.) to the wound site and/or injured tissue.

The term “extracellular matrix” refers to the secretion by cells of fibrous elements (e.g., collagen, elastin, reticulin), link proteins (e.g., fibronectin, laminin), and space filling molecules (e.g., glycosaminoglycans). The extracellular matrix may contain additional components such as proteins and other molecules which may or may not be unique to each tissue

The term “extracellular matrix deposition” refers to the secretion by cells of fibrous elements (e.g., collagen, elastin, reticulin), link proteins (e.g., fibronectin, laminin), and space filling molecules (e.g., glycosaminoglycans). As used herein, the term “type I collagen” refers to the most abundant collagen, which forms large well-organized fibrils having high tensile strength.

The term “re-epithelialization” refers to the reformation of epithelium over a denuded surface (e.g., wound and/or injured tissue).

The term “remodeling” refers to the replacement of and/or devascularization of granulation tissue.

The term “regeneration” refers to the conversion of the injured tissue with structurally and functionally healthy tissue similar to the tissue prior to injury. The term “impaired healing capabilities” comprises wounds, which are characterized by a disturbed wound healing process. Examples of wounds with impaired healing capabilities are wounds of diabetic patients and alcoholics, wounds which are infected by microorganisms, ischemic wounds, wounds of patients suffering from deficient blood supply or venous stasis, and ulcers. Particularly preferred wounds are diabetic wounds. Other preferred wounds include wounds of elderly subjects and chronic wounds of subjects of any age.

As used herein, the term “chronic wound” refers to a wound that does not fully heal even after a prolonged period of time (e.g., 2 to 3 months or longer).

The term “diabetic wounds” refers to wounds of mammals and humans suffering from diabetes. An example of a diabetic wound is an ulcer (e.g., Ulcus cruris arteriosum or Necrobiosis lipoidica).

As used herein, the term “ulcer” (i.e., “ulceration”) refers to a local defect or excavation of the surface of an organ or tissue, produced by sloughing of necrotic tissue. The term encompasses various forms of ulcers (e.g., diabetic, neuropathic, arterial, decubitus, dental, perforating, phagedenic, rodent, trophic, tropical, varicose, venereal, etc.), although in preferred embodiments, surface (i.e., skin) ulcers are involved in the present invention. Especially preferred ulcers are diabetic ulcers.

In some embodiments, the present invention provides methods and compositions for “accelerating wound healing,” whereby different aspects of the wound healing process are “enhanced.” As used herein, the term “enhanced” indicates that the methods and compositions provide an increased rate of wound healing. In preferred embodiments, the term “enhanced” indicates that the wound healing rate and/or a wound healing process occurs at least 10% faster than is observed in untreated or control-treated wounds. In particularly preferred embodiments, the term “enhanced” indicates that the wound healing rate and/or a wound healing process occurs at least 15% faster than is observed in untreated or control-treated wounds. In still further preferred embodiments, the term “enhanced” indicates that the wound healing rate and/or a wound healing process occurs at least 20% (e.g., 50%, 100%, . . . ) faster than wounds untreated or control-treated wounds.

As used herein, the terms “localized” and “local” refer to the involvement of a limited area. Thus, in contrast to “systemic” treatment, in which the entire body is involved, usually through the vascular and/or lymph systems, localized treatment involves the treatment of a specific, limited area. Thus, in some embodiments, discrete wounds and/or injuries are treated locally using the methods and compositions of the present invention.

The term, “VEGF” as used herein, is an art accepted abbreviation for vascular endothelial growth factor.

As used herein, the term “topically” means application to the surface of the skin, mucosa, viscera, etc. Similarly, the terms “topically active drug” and “topically active agent” refer to a substance or composition, which elicits a pharmacologic response at the site of application (e.g., skin), but is not necessarily an antimicrobial agent.

As used herein, the term “medical devices” includes any material or device that is used on, in, or through a patient's body in the course of medical treatment for a disease or injury. Medical devices include, but are not limited to, such items as medical implants, wound care devices, injured tissue care devises, drug delivery devices, and body cavity and personal protection devices. The medical implants include, but are not limited to, injections, urinary catheters, intravascular catheters, dialysis shunts, wound drain tubes, injured tissue drain tubes, skin sutures, vascular grafts, implantable meshes, intraocular devices, heart valves, and the like.

As used herein, “stem cells” include, but are not limited to undifferentiated biological cells that can differentiate into specialized cells and can divide (through mitosis) to produce more stem cells. The term “stem cells” includes embryonic stem cells, which are isolated from the inner cell mass of blastocysts, and adult stem cells, which are found in various tissues and stem cells from the cord blood. Stem cells and progenitor cells in adults act as a repair system for the body, replenishing adult tissues. In a developing embryo, stem cells can differentiate into all the specialized cells—ectoderm, endoderm and mesoderm (see induced pluripotent stem cells)—but also maintain the normal turnover of regenerative organs, such as blood, skin, or intestinal tissues. The term stem cells also includes normal mature cells that underwent treatment such as acid shock or stable transfection with various genes in order to convert these cells into pluripotent stem cells. Stem cells may originate from autologous tissues, allogeneic tissues or xenogeneic tissues.

As used herein, “wound care devices” and “burn care devices” include, but are not limited to conventional materials such as dressings, plasters, compresses, ointments containing the pharmaceuticals, or gels containing the pharmaceuticals that can be used in accordance with the present invention. Thus, it is possible to administer the wound care devices comprising α-gal epitopes or α-gal epitopes and anti-Gal antibodies topically and locally in order to exert an immediate and direct effect on wound healing. The topical administration of wound care devices can be effected, for example, in the form of a solution, an emulsion, a cream, an ointment, a foam, an aerosol spray, a gel matrix, a sponge, drops or washings.

As used herein, “α-gal nanoparticles suspension” include, but are not limited to conventional suspensions of α-gal nanoparticles in a fluid aqueous vehicle such as, but not limited to, saline (physiological sodium chloride solutions), phosphate buffered saline, or any other fluid or gel. Suitable additives or auxiliary substances are isotonic solutions, such as physiological sodium chloride solutions or sodium alginate, demineralized water, stabilizers, collagen containing substances such as Zyderm II or matrix-forming substances such as povidone and collagen sheets. To generate a gel basis, formulations, such as aluminum hydroxide, polyacrylacid derivatives, collagen, and cellulose derivatives (e.g., carboxymethyl cellulose), fibrin, and plasma clots are suitable. These gels can be prepared as hydrogels on a water basis as polyethylene glycol (PEG) or as oleogels with low and high molecular weight paraffines or Vaseline and/or yellow or white wax. As emulsifier alkali soaps, metal soaps, amine soaps or partial fatty acid esters of sorbitants can be used, whereas lipids can be added as Vaseline, natural and synthetic waxes, fatty acids, mono-, di-, triglycerides, paraffin, natural oils or synthetic fats. The wound care and/or injured tissue devices comprising α-gal epitopes and anti-Gal antibodies according to the invention can also, where appropriate, be administered topically and locally, in the region of the wound and/or injured tissue, in the form of liposome/antibody complexes, nanoparticle/antibody complexes, or complexes between any antigen and its corresponding antibody, or complement activating substances.

Furthermore, the treatment can be effected using a transdermal therapeutic system (TTS), which enables the pharmaceuticals of the present invention to be released in a temporally controlled manner. To improve the penetration of the administered drug through the membrane, additives such as ethanol, urea or propylene glycol can be added in addition to polymeric auxiliaries.

The term “fibrin clot” refers to any mass, mesh, plug comprising isolated fibrinogen mixed with thrombin and thus induced to convert into fibrin that is non globular and forms a clot.

The term “plasma clot” refers to plasma mixed with an agent inducing conversion of fibrinogen within the plasma into non globular fibrin, thereby forming a clot.

The term “soluble” refers to any ability of a compound to completely dissolve within a solution. Usually, but not exclusively, the compound may be a salt that dissociates into a cationic and anionic species. Nonetheless, it would be expected that a fully soluble compound comprises a monomeric species.

The term “physiological composition” or “pharmaceutical composition” as used herein, are clinically acceptable (i.e., for example, antiseptic, sterile, non-inflammatory, non-allergenic) such they can be administered internally and/or externally and may comprise any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such compositions.

The term “semi-solid filler” refers to gel or gel-like materials that can include α-gal nanoparticles or α-gal carrying molecules and can fill various spaces in the body. The gel consistency of the semi-solid filler enables the diffusion in it of antibodies such as the anti-Gal antibody, complement and other proteins for the recruitment of macrophages into areas where the semi-solid filler is applied. Non-limiting examples for semi-solid fillers are plasma clot, hydrogel and fibrin glue.

The term “biomaterial” refers to any material that is used for implantation and is of synthetic source or natural source. It further includes tissues and organs from various species which may or may not undergo decellularization process and/or crosslinking treatment for the purpose of implantation into patients in order to induce repair and regeneration of injured or nonfunctioning tissues and organs.

The term “decellularization” refers to a process that involves immersion or perfusion of a tissue or organ in detergent solutions that solubilize cell membranes, remove cells and nuclei in order to produce a tissue or organ implant containing extracellular matrix without live cells.

The term “PVA sponge disc” refers to a sponge disc (10 mm in diameter and 3 mm in thickness) made of biological inert material called polyvinyl alcohol (PVA) which is used for subcutaneous implantation in mice and which can contain saline, α-gal nanoparticles suspension and/or suspension of various fragmented tissue homogenate (e.g. cartilage homogenate).

### DETAILED DESCRIPTION OF THE INVENTION

The present invention is related to the field of wound healing, tissue regeneration and tissue engineering by various synthetic and natural compositions. In particular, the present invention provides compositions and methods comprising molecules with linked α-gal epitopes for induction of and/or macrophage recruitment and pro-healing activation localized within or surrounding damaged tissue. In some embodiments, the present invention provides treatments for tissue repair in normal subjects and in subjects having impaired healing capabilities, such as diabetic and aged subjects.

In some embodiments, the invention relates to methods and compositions for the promotion of wound healing. Macrophages play a major role in the success of wound healing in part by generation of reactive radicals such as nitric oxide and oxygen peroxide, and through the secretion of collagenase and elastase as provided for in Bryant et al., Prog. Clin. Biol. Res. 266, 273 (1988) and Knighton et al., Prog. Clin. Biol. Res. 299, 217 (1989), both of which are hereby incorporated by reference. Macrophages secrete cytokines and growth factors that are essential in recruitment of macrophages, lymphocytes, stem cells and fibroblasts into the wound site. Cytokines and growth factors also regulate fibroblast and epithelial cell proliferation, as well as proliferation of endothelial cells for revascularization as disclosed in Rappolee and Werb, Curr. Top. Microbiol. Immunol. 181, 87 (1992) and Nathan, J. Clin. Invest. 79, 319 (1987), both of which are hereby incorporated by reference. Accordingly, experiments in macrophage-depleted animals have been associated with defects in wound healing as provided for in Leibovich and Ross, Am. J. Pathol. 78, 71 (1975), incorporated in its entirety by reference.

Accelerated wound healing and improved repair and remodeling of damaged tissues is contemplated to be achievable by effectively controlling recruitment of monocytes and differentiation of these cells into activated macrophages. Activated macrophages in turn secrete fibrogenic and angiogenic growth factors inducing formation of granulation tissue containing myofibroblasts as described in Frangogiannis, Curr. Med. Chem. 13, 1877 (2006), incorporated herein by reference, and angiogenesis associated with local collagen synthesis and re-epithelization as provided for in Stein and Keshav, Clin. Exp. Allergy 22, 19 (1992); DiPietro, Shock 4, 233 (1995); Clark, J. Dermatol. Surg. Oncol. 19, 693 (1993) and Rappolee and Werb, Curr. Topics Microbial Immunol. 181, 87 (1992), all of which are hereby incorporated by reference. Macrophages have key functions in almost every stage of the wound healing, tissue repair and remodeling processes. Upon initiation of the inflammatory stage, macrophages secrete interleukin-1 (IL-1), which induces the rapid recruitment of inflammatory cells from the circulation into the wound as provided for in Dinarello, FASEB J. 2, 108 (1988), incorporated in its entirety by reference. As phagocytes, the macrophages aid in the digestion of bacteria and cell debris as described in Aderem et al., Ann. Rev. Immunol. 17, 593 (1999), incorporated herein by reference. In later stages, macrophages secrete interleukin-6 (IL-6), which influences endothelial cell proliferation and initiation of angiogenesis as discussed in Mateo et al., Am. J. Physiol. 266, R1840 (1994), hereby incorporated by reference. Macrophages further coordinate cellular proliferation by production of growth factors such as α and β vascular endothelial cell growth factors (VGEF), epidermal growth factor (EGF), fibroblast growth factor (FGF) and insulin-like growth factor (IGF) as provided for in Singer et al., New England Journal of Medicine 341, 738 (1999), incorporated herein by reference. Moreover, local administration of in vitro activated macrophages into ulcerated wounds, or into wounds resulting from infections following open heart surgery, was found to accelerate the wound healing process as described in Danon et al., Exp. Gerontol. 32, 633 (1997) and Orensterin et al., Wound Repair Regen. 13, 237 (2005), both of which are incorporated in their entirety by reference.

An additional characteristic attributed to recruited macrophage is the ability of a small proportion of them to function as stem cells. It has been reported that monocytes/macrophages include a small population of multipotential stem cells that can proliferate and undergo transdifferentiation into various types of cells, based on microenvironment and on the adjacent cells as indicated by Seta et al., Keio J Med. 56:41 (2007). For example, incubation of human macrophages in presence of chicken cardiomyocytes results in differentiation of a small proportion of cells into human cardiomyocytes. Similarly, incubation of human macrophages with rat fetal neurons results in induction of human stem cells among macrophages to differentiate into neurons. Macrophages are capable of recruiting stem cells from the adjacent uninjured tissue or from other sites in the body, and/or macrophages trans-differentiate into stem cells. The recruitment and activation by the treatment of injection of α-gal liposomes and/or α-gal nanoparticles into injured tissues results in rapid migration of stem cells into treated injured tissue and in accelerated repair and regeneration of the injured tissue for the restoration of its pre-injury biological activity. As illustrated in FIGS. 1 and 29, this recruitment and activation is the result of the interaction between the natural anti-Gal antibody and α-gal epitopes on the administered α-gal liposomes and/or α-gal nanoparticles (i.e. α-gal liposomes of submicroscopic size) and the subsequent Fc/FcγR interaction between macrophages and the anti-Gal bound to α-gal liposomes and/or α-gal nanoparticles.

In some embodiments, the present invention provides for compositions and methods for using the anti-Gal antibody for the recruitment and local activation of neutrophils, monocytes and macrophages within and adjacent to wounded tissue. This is achieved by administration of compositions comprising liposomes bearing multiple α-gal epitopes (Galα1-3Galβ1-(3)4GlcNAc-R) as part of the glycolipid component. The anti-Gal antibody, which constitutes 1% of immunoglobulins in humans, apes, Old World primates and birds, interacts specifically with α-gal epitopes. In situ binding of anti-Gal to α-gal epitopes on α-gal glycolipids and to other molecules carrying this epitope, results in local activation of complement and generation of the chemotactic factors C5a, C4a and C3a. These factors direct migration of neutrophils followed by monocytes and macrophages into the injection site. These inflammatory infiltrates are suitable for combating microbes within infected wounds. In addition, the monocytes and macrophages infiltrates are contemplated to bind by their Fcγ receptors, anti-Gal antibodies via the Fc portion of anti-Gal opsonizing the α-gal liposomes, thereby activating these cells. This in turn induces the uptake of the anti-Gal opsonized α-gal liposomes and the secretion of cytokines and growth factors that accelerate wound healing (FIG. 1). As such treatment regimens comprising α-gal liposomes administration within and/or adjacent to a wound are contemplated to result in accelerated healing and improved repair of damaged tissues. Alternatively, topical application of ointments containing α-gal glycolipids (referred to as α-gal ointments) results in similar binding of anti-Gal to α-gal glycolipids, complement activation, chemotactic migration of neutrophils, monocytes and macrophages into the treated area, activation of these macrophages and local secretion of cytokines and growth factors by these macrophages that contribute to accelerated wound healing.

When a wound occurs to the skin, the cells must work to close the breach and re-establish the barrier to the environment. The process of wound healing typically consists of three phases during which the injured tissue is repaired, regenerated, and new tissue is reorganized into a scar. These three phases can be classified as: a) an inflammation phase which begins on day 0 and lasts up to 3 days; b) a cellular proliferation phase from 3 to 12 days; and c) a remodeling phase from 3 days to about 6 months.

In the inflammation phase, inflammatory cells, mostly neutrophils, enter the site of the wound followed by lymphocytes, monocytes, and later macrophages. Stimulated neutrophils release proteases and reactive oxygen species into the surrounding medium, with potential adverse effects on both the adjacent tissues and the invading microorganisms. The proliferative phase consists of laying down new granulation tissue, and the formation of new blood vessels in the injured area. Fibroblasts, endothelial cells, and epithelial cells migrate to the wound site. These fibroblasts produce the collagen necessary for wound repair. In re-epithelialization, epithelial cells migrate from the free edges of the tissue across the wound. This event is succeeded by the proliferation of epithelial cells at the periphery of the wound. In general, re-epithelialization is enhanced by the presence of occlusive wound dressings that maintain a moisture barrier. Remodeling, the final phase of wound healing, is effected by both the replacement of granulation tissue with collagen and elastin fibers and the devascularization of the granulation tissue. Eventually, in most cases, a scar forms over the wounded area. Application of α-gal liposomes and/or α-gal nanoparticles accelerates this wound healing process the extent that the normal architecture of the skin is restored and scar formation is avoided.

The present invention teaches how to use α-gal liposomes with the preferred size range but not limited to 0.1-1000 μm and submicroscopic α-gal liposomes with the size range of 0.0001-0.5 μm, called here “α-gal nanoparticles”, for inducing recruitment and activation of macrophages in injured internal tissues. For simplicity of the text both α-gal liposomes and α-gal nanoparticles are referred to in this application as α-gal nanoparticles since they are comprised of the same components and have the same spherical structure (FIG. 30). It is contemplated that the recruitment and activation of macrophages by α-gal nanoparticles accelerates physiologic processes that result in repair and regeneration of the treated injured tissues into which α-gal nanoparticles are applied. The present invention further teaches the use of α-gal nanoparticles in improving the efficacy of biomaterials used for tissue engineering such as, but not limited to, collagen sheets, decellularized tissues and decellularized organs, by introducing α-gal nanoparticles into these biomaterials. Upon implantation of such biomaterials in patients the α-gal nanoparticles will induce rapid recruitment of macrophages into the implant and activation of these recruited macrophages within the implant. It is contemplated that this recruitment and activation of macrophages by α-gal nanoparticles will initiate physiologic processes that result in effective repair and regeneration of the implant which ultimately will convert it into biologically functioning tissue or organ.

Macrophages are the pivotal cells in early stages of injury healing and tissue regeneration. Macrophages migrating into injury sites debride the injured tissue by phagocytosis. Macrophages debride the tissue of dead cells. Subsequently, upon transition into the pro-healing phase, macrophages orchestrate regeneration by secreting a variety of cytokines/growth factors that facilitate the repair and regeneration processes in the injured tissue and regeneration of the injured tissue (Bryant et al., Prog Clin Biol Res, 266:273, 1988; Knighton and Fiegel, Prog Clin Biol Res, 299:217, 1989; DiPietro, Shock 4:233, 1995). Macrophages also secrete cytokines and growth factors that are essential in further recruitment of macrophages, as well as recruitment of stem cells into the injury site (Lolmede et al. J Leukoc Biol 85: 779, 2009). Cytokines and growth factors also regulate fibroblast and epithelial cell proliferation, as well as proliferation of endothelial cells for revascularization of the injured tissue (Rappolee and Werb, Curr Top Microbiol Immunol, 181:87, 1992). Compositions and methods to inducing rapid recruitment of macrophages into injured tissues, thereby accelerate the repair and regeneration of these tissues and are desirable for decreasing morbidity and for achieving a more effective repair of injured tissues than the post injury physiologic repair.

Macrophages are physiologically recruited into wounds within several days by cytokines such as MIP-1, MCP-1 and RANTES released from cells within and around injury sites (Low et al. Am J Pathol 159: 457, 2001; Heinrich et al. Wound Repair Regen, 11: 110, 2003; Shukaliak and Dorovini-Zis J Neuropathol Exp Neurol 59: 339, 2000). This recruitment of macrophages into skin wounds and internal injured tissues can be markedly accelerated by antibodies interacting with various antigens and causing local activation of the complement system. Complement activation results in generation of complement cleavage peptides such as C5a, C4a and C3a which are chemotactic factors that induce rapid extravasation of monocytes, and their differentiation into macrophages which migrate along the chemotactic gradient (Snyderman and Pike Annu Rev Immunol 2:257, 1984; Haeney J Antimicrobial Chemother 41, 41, 1998).

Some studies indicated that macrophages also include populations of pluripotent/pluripotential stem cells (Seta and Kuwana, Keio J. Med 56: 41, 2007). Other studies demonstrated recruitment of stem cells by macrophages that reach the injury sites (Lolmede et al. supra). Whether stem cells are recruited by macrophages or originate from macrophages, upon reaching the injured area, they receive instructive cues from differentiated cells located nearby, from the extracellular matrix (ECM) and from the microenvironment, which direct the stem cells to differentiate into functional cells that repair and regenerate the injured tissue (Seta and Kuwana, supra; Stappenbeck and Miyoshi, Science, 324: 1666, 2009). If stem cells do not reach the injury site early enough after the injury, then the default repair mechanism which takes place is fibrosis mediated by fibroblasts that infiltrate the injury site. Therefore, accelerated and improved repair and regeneration of damaged tissues is contemplated to be achievable by therapeutic induction for recruitment of monocytes and macrophages into the injured tissue, induction of the recruited monocytes to mature into macrophages and activation of the recruited macrophages and those recruited monocytes differentiating into macrophages to further mature into macrophages that promote tissue repair. Such macrophages in turn secrete a variety of cytokines and growth factors that promote angiogenesis, as well as induce the recruitment and activation of stem cells that repopulate the injured or damaged tissue, support the survival of the recruited stem cells in the injury site and regenerate the injured or damaged tissue for restoration of the pre-injury biological activity (Stein and Keshav, Clin Exp Allergy 22:19, 1992; DiPietro, supra; Clark, J Dermatol Surg Oncol, 19:693, 1993; and Rappolee and Werb, supra; Allison and Islam J. Pathol 217: 144, 2009; Lesault et al. PLoS One 7: e46698, 2012). In view of these observations, it can be contemplated that a method for rapid recruitement of macrophages into internal injury sites and the activation of these macrophages to secrete cytokines and growth factors that recruit stem cells facilitating healing of injured tissues will be helpful in treatments for repair and regeneration of tissues and organs following various types of injuries or damages to tissues.

The present invention teaches methods for rapid recruitment and activation of monocytes and macrophages within injured internal human tissues and engineered tissues by harnessing the immunological potential of the natural anti-Gal antibody. The harnessing of this antibody is feasible by the use of α-gal nanoparticles. Injection or application of α-gal nanoparticles by various methods into ischemic myocardium post infarction induces rapid recruitment of macrophages to the injection site and local activation of the recruited macrophages. These activated macrophages further recruit stem cells that will be guided by preserved dead cardiomyocytes and the preserved extracellular matrix (ECM) to differentiate into functional cardiomyocytes, thereby restoring the biological activity of the injured myocardium. In the absence of rapid and extensive recruitment and activation of macrophages by α-gal nanoparticles treatment, the default mechanism of fibrosis of the ischemic myocardium will result in permanent prevention of regeneration of the injured site into functional myocardium.

The invention further describes how the angiogenesis induced by macrophages that are recruited and activated by α-gal nanoparticles administered into nerve injury sites may further induce effective axonal sprouting in order to bridge the neural lesion area. Such sprouting axons grow into the post lesion axonal tube and regenerate the nerve. In the absence of rapid and extensive recruitment and activation of macrophages by α-gal nanoparticles treatment, the default mechanism of fibrosis of the injured nerve will result in permanent prevention of regeneration of the injured nerve. A similar injection or administration of α-gal nanoparticles to other internal injuries will induce the rapid recruitment and activation of macrophages which ultimately may accelerate and improve the efficacy of the repair and regeneration mechanism for restoring the biological activity of the treated injured tissues including, but not limited to joint articular and meniscus cartilage, bone, lung, brain, nerve tissue, skeletal muscle, heart muscle. smooth muscle, epidermal tissues and other epithelial tissues, connective tissue, endocrine and exocrine glands, urinary bladder, blood vessels and other duct tissues, gastrointestinal tract, eye, ear, limbs and various organs.

This invention also describes methods for the incorporation of α-gal nanoparticles into biomaterials which are used in tissue engineering and which will increase the efficacy of such biomaterials in regeneration of injured tissues. The α-gal nanoparticles placed within biomaterials such as, but not limited to, collagen sheets and decellularized tissues and organs will induce rapid and extensive recruitment of macrophages upon implantation of such biomaterials. The α-gal nanoparticles will further induce activation of these recruited macrophages which, in turn, will produce cytokines and growth factors that will induce effective recruitment of stem cells into the implanted biomaterials. When natural decellularized tissues and organs containing α-gal nanoparticles are used for implantation, the recruited stem cells into these implants will receive guidance from the microenvironment and extracellular matrix scaffold for the differentiation into cells that repopulate the biomaterial and restore biological activity of the damaged tissue or organ replaced by the biomaterial.

The invention also describes the administration of α-gal nanoparticles together with stem cells or with cells treated to convert into stem cells in order to improve the stem cell treatment efficacy in regenerating tissues. The recruitment and activation of macrophages into area of administered stem cells results in localized secretion of cytokines and growth factors by the macrophages that ultimately generates a microenvironment that supports growth and differentiation of the administered stem cells into the cells that repair the injured or damaged tissue.

This invention teaches methods for using α-gal nanoparticles in order to induce rapid recruitment of macrophages into internal tissues that are injured and induce activation of these macrophages to produce cytokines and growth factors. The secreted cytokines and growth factors facilitate recruitment of stem cells and repair and regeneration of the injured tissue. This invention further teaches the application of α-gal nanoparticles into biomaterials including, but not limited to decellularized tissues and organs, in order to induce rapid recruitment of macrophages into biomaterials once they are implanted and in order to induce activation of the recruited macrophages so these cells produce cytokines and growth factors that recruit stem cells and improve the efficacy and pace of regeneration of biomaterials implanted for the purpose of tissue engineering.

α-Gal nanoparticles are nanoparticles which present multiple α-gal epitopes and their size usually range from 0.0001-0.5 μm. Decreasing the size of α-gal liposomes by additional sonication to the size range of 0.0001-0.5 μm enables their effective sterilization by filtration through filters that remove bacteria, thereby increasing the safety of these particles in clinical use. For simplicity purpose both α-gal nanoparticles and α-gal liposomes are referred to in the present application as α-gal nanoparticles and they encompass particles that present multiple α-gal epitopes and have a size range of 0.0001-1000 μm.

The α-gal epitope is a carbohydrate antigen with the structure Galα1-3Galβ1-4GlcNAc-R that binds the human natural anti-Gal antibody. Anti-Gal is the most abundant natural antibody in all humans, constituting ˜1% of immunoglobulins (reviewed in Galili U. Immunology 140: 1, 2013). Anti-Gal binding to α-gal nanoparticles that are introduced into injured tissues, or to α-gal nanoparticles within implanted biomaterials, activates the complement system thereby generating chemotactic complement cleavage peptides that induce rapid and extensive recruitment of macrophages. The subsequent interaction between Fc portion of anti-Gal coating α-gal nanoparticles and Fcγ receptors on macrophages activates these cells to produce cytokines and growth factors (also referred to as cytokines/growth factors) that promote injury repair and recruit stem cells that repopulate the tissue and the implant with cells that restore the biological activity of the tissue. Similarly, recruitment of stem cells by activated macrophages within the engineered tissue implant or organ will result in effective regeneration of the tissues within the implant.

In one embodiment, α-gal nanoparticles injected into ischemic myocardium induce extensive recruitment of macrophages that are activated to secrete cytokines/growth factors that preserve the structure of the ischemic tissue. These cytokines/growth factors further recruit stem cells into the ischemic myocardium. The recruited stem cells are guided by the microenvironment and the preserved extracellular matrix (ECM) to differentiate into cardiomyocytes that repopulate the ischemic myocardium and restore its biological activity.

In another embodiment the α-gal nanoparticles are applied to nerve injury sites where part or all the nerve axon severed or injured, such as in spinal cord injuries. These α-gal nanoparticles recruit macrophages into the nerve injury site. The recruited macrophages secrete cytokines/growth factors including, but not limited to vascular endothelial growth factor (VEGF) that induce local angiogenesis. This angiogenesis promotes axonal sprouting. The axon sprouts grow across the neural lesion area and into the post lesion axonal tube. This bridging of the axonal sprouts across the neural lesion into the post lesion axonal tube ultimately results in the regeneration of the injured nerve. In the absence of rapid and extensive recruitment and activation of macrophages by α-gal nanoparticles, the default mechanism of fibrosis will occur in the nerve lesion and thus, will result in permanent prevention of regeneration of the injured nerve. A similar injection or application of α-gal nanoparticles in other internal injuries will induce the rapid recruitment and activation of macrophages (as illustrated in FIG. 29) which ultimately may accelerate and improve the efficacy of the repair and regeneration mechanism for restoring the structure and biological activity of the treated injured tissues.

In another embodiment, α-gal nanoparticles can facilitate the recruitment and activation of macrophages within injured lungs in order to facilitate repair and regeneration of injured lungs. A human lung was found to be able to grow after pneumonectomy i.e. resection of the lung for the removal of malignancy (Butler et al. New Engl. J. Med. 367: 244, 2012). Accordingly, stem cells with a regenerative ability were found in adult human lungs (Wansleeben et al. Wiley Interdiscip Rev Dev Biol. 2: 131, 2013; Foronjy and Majka Cells 1: 874, 2012). This ability of lungs to regenerate due to the activity of stem cells within them makes lungs amenable to treatment with α-gal nanoparticles. Administration of α-gal nanoparticles in an aerosolized suspension into lungs damaged by smoking, asbestosis or other injuries, or into lungs that were partly resected and the interaction of these nanoparticles with the anti-Gal antibody will induce chemotactic recruitment of macrophages onto the surface of damaged alveoli and airways and activation of these macrophages. The macrophages will secrete cytokines/growth factors that induce recruitment and activation of stem cells as well as form a microenvironment that is conducive to the activity of the recruited stem cells. It is contemplated that under such conditions the recruited stem cells within the damaged alveoli will differentiate into pneumocytes that regenerate the damaged alveoli (air sacs) and/or form new alveoli. Within the bronchioles, bronchi and trachea, the macrophages recruited and activated by α-gal nanoparticles will recruit stem cells that differentiate into the ciliated epithelium and mucus secreting cells that comprise the normal epithelium of the airways.

In one embodiment a paste/gel containing the α-gal nanoparticles, or α-gal nanoparticles and cartilage fragments with the size range of 0.01-5000 μm can be applied in areas of articular cartilage defects in joints. The interaction of the α-gal nanoparticles with the anti-Gal antibody activates the complement system proteins diffusing into the applied paste/gel and generate complement cleavage chemotactic factors. These chemotactic factors recruit macrophages which are activated following binding the Fc portion of anti-Gal on the α-gal nanoparticles. The activated macrophages secrete cytokines/growth factors which recruit stem cells. The stem cells recruited into the applied paste/gel are instructed by the cartilage ECM fragments to differentiate into chondroblasts and subsequently into chondrocytes producing the regenerating cartilage.

In another embodiment a paste/gel containing the α-gal nanoparticles, or α-gal nanoparticles and bone fragments with the size range of 0.01-5000 μm is applied to sites of bone fractures or into bone resection sites where it mediates accelerated regeneration of the injured bone. The interaction of the α-gal nanoparticles with the anti-Gal antibody activates the complement system proteins diffusing into the applied paste/gel and generate complement cleavage chemotactic factors. These chemotactic factors recruit macrophages which are activated following binding the Fc portion of anti-Gal on the α-gal nanoparticles. The activated macrophages secrete cytokines/growth factors which recruit stem cells. The stem cells recruited into the applied paste/gel differentiate into osteoblasts producing the regenerating bone tissue.

In another embodiment, incorporation of α-gal nanoparticles into biomaterials which are used in tissue engineering will increase the efficacy of such biomaterials in facilitating regeneration of injured tissues. The α-gal nanoparticles are placed within biomaterials such as, but not limited to, collagen sheets and decellularized tissues and organs such as decellularized liver, heart, kidney, intestine, stomach, striated muscle, heart muscle, smooth muscle, esophagus, urinary bladder, cartilage, bone, connective tissue or any other decellularized organ or tissue. Implantation of biomaterials or decellularized tissues or organs containing α-gal nanoparticles into patients will result in binding of the natural anti-Gal antibody of the treated patient to the α-gal epitopes on these nanoparticles. This will result in activation of the complement system and thus generation of complement cleavage chemotactic peptides that induce rapid and extensive recruitment of macrophages into the implant. The α-gal nanoparticles will further induce activation of these recruited macrophages by the interaction between anti-Gal antibody molecules bound to these nanoparticles and Fcγ receptors on the macrophages. These activated macrophages will produce cytokines/growth factors that will induce effective recruitment of stem cells into the implanted biomaterials. When natural decellularized tissues and organs containing α-gal nanoparticles are used for implantation, or when biomaterials containing ECM are used as implants, the stem cells recruited into these implants will receive guidance from the microenvironment and ECM scaffold for the differentiation into cells that repopulate the biomaterial and restore biological activity of the damaged tissue or organ replaced by the biomaterial.

In another embodiment, macrophages recruited and activated by α-gal nanoparticles can support the viability and function of administered stem cells and of mature cells converted into stem cells by various methods such as but not limited to acid shock (Okobata et al. Nature 505: 641, 2014) and introduction of various genes. When administered into injured sites, the stem cells and mature cells converted into stem cells may survive for periods of time that are not long enough to enable conversion into the cells that regenerate the injured tissue. When such stem cells are administered within a suspension also containing α-gal nanoparticles, the interaction of the administered α-gal nanoparticles with the anti-Gal antibody activates the complement system proteins diffusing into the site of injected stem cells and of cells converted into stem cells. The anti-Gal/α-gal nanoparticles interaction and ensuing complement activation generate complement cleavage chemotactic factors. These chemotactic factors recruit macrophages which are activated following binding the Fc portion of anti-Gal on the α-gal nanoparticles. The activated macrophages secrete cytokines/growth factors which facilitate the survival of the stem cells and of cells converted into stem cells for periods long enough to enable their effective differentiation into cells that regenerate the injured tissue.

### I. The Role of Inflammatory Cells in Wound Healing and Tissue Repair

Neutrophils are the first immune cells to arrive at the wound site appearing approximately 24 h after injury. They phagocytose bacteria and mediate wound debridement. Macrophages migrate into the wound 48-96 h after injury and become the predominant cells within the inflammatory response in the wound. Studies on depletion of monocytes and/or macrophages in mice by intravascular administration of specific anti-macrophage antibodies have indicated that wound healing is impaired after depletion of these cells as provided for in Leibovich et al., Am. J. Pathol. 78, 71 (1975), incorporated herein by reference. In contrast, depletion of granulocytes, including neutrophils, through the use of specific anti-granulocyte antibodies does not hamper the inflammatory response and subsequent wound healing and tissue repair as provided for in Leibovich et al., Am. J. Pathol. 78, 71 (1975), incorporated herein by reference. This result suggests that cells of the monocyte/macrophage lineage are pivotal in orchestrating wound healing and tissue repair and in remodeling following injury. As such the present invention provides compositions and methods for inducing rapid recruitment of macrophages into wounds and injured tissues to accelerate the process of wound healing and tissue repair. Circulating monocytes enter the wound and mature into macrophages and dendritic cells. They secrete interferon-γ (IFNγ), and angiogenic and fibrogenic growth factors. These factors and additional chemokines, cytokines and growth factors that are produced after debridement of the injured tissue and are instrumental in the removal of dead cells, localized recruitment of fibroblasts and stem cells, cell proliferation and tissue remodeling to effect wound healing. This tissue repair process occurs in infected wounds, surgical incisions, burns and other traumatized tissues as disclosed in Rappolee and Werb, Curr. Top. Microbiol. Immunol. 181, 87 (1992); Nathan, J. Clin. Invest. 79, 319 (1987) and Singer et al., New England Journal of Medicine 341, 738 (1999), all of which are hereby incorporated by reference. Major chemoattractants directing migration of neutrophils, monocytes and macrophages are the C5a and C3a fragments of the complement components C5 and C3, which are generated following complement activation by antigen/antibody interactions. These chemotactic factors form a concentration gradient that guides the migration of neutrophils, monocytes and macrophages to the areas with increased concentrations of C5a and C3a.

In one embodiment, the present invention provides compositions and methods for inducing rapid recruitment of macrophages into the injured tissues and into biomaterial implants and for activation of the recruited macrophage. This invention teaches how to recruit and activate macrophages by injection, or by other means of delivery, a preparation comprising nanoparticles presenting an α-gal epitope having a terminal α-galactosyl to an injured tissue of a subject having endogenous anti-Gal antibody, to produce a treated injured tissue. In some embodiments, the terminal α-gal is selected from the group consisting of Galα1-3Gal-R, Galα1-2Gal-R, Galα1-6Gal-R and Galα1-6Glc-R. The α-gal epitopes on the nanoparticles further include commercially available oligosaccharides available from companies such as, but not limited to, Dextra (UK), Elicityl (France), Vector and Sigman (USA).

In some embodiments, the present invention provides for compositions and methods for the recruitment and activation of large numbers of neutrophils, monocytes and macrophages into wounds by local injection of liposomes possessing multiple α-gal epitopes (Galα1-3Galβ1-4GlcNAc-R or Galα1-3Galβ1-3GlcNAc-R) on their glycolipid components, or by topical application of ointment containing α-gal glycolipids. The α-gal epitopes bind the natural anti-Gal antibody, which is the most abundant antibody in humans. This antigen/antibody interaction in turn activates complement forming the degradation products C5a and C3a that serve as effective chemoattractants for inflammatory cells.

In some preferred embodiments, the α-gal epitope is part of a molecule selected from the group consisting of a glycolipid (e.g., α-gal epitopes on carbohydrate chain that is linked to ceramide), a glycoprotein (e.g., α-gal albumin), a proteoglycan, a glycopolymer (e.g., α-gal polyethylene glycol) and any other natural or synthetic spacer. In some particularly preferred embodiments, the glycolipid comprises α-gal nanoparticles in which the nanoparticles have on their surface α-gal epitopes that are capable of binding the anti-Gal antibody. In some embodiments the α-gal epitopes are presented on any type of nanoparticles prepared from synthetic and/or natural materials. Also provided are methods in which the preparation further comprises anti-Gal antibodies bound to the α-gal nanoparticles. In some preferred embodiments, the preparation is part of a tissue repair device selected from the group consisting of α-gal nanoparticles suspension, gels, semi-permeable films, ointments, foams, synthetic biomaterials, natural biomaterials including decellularized tissues and decellularized organs such as, but not limited to, heart tissue liver organ, intestinal tissue and urinary bladder tissue or any other tissue or organ. In some embodiments the α-gal nanoparticles are of large size visible in a microscope and may be referred to as α-gal liposomes.

In some embodiments, the glycolipid preparation comprising α-gal nanoparticles is derived from a source selected from the group consisting of rabbit red blood cells, bovine red blood cells, and other non-primate mammalian cells. In another embodiment the glycolipid preparation comprising α-gal nanoparticles. In addition, the present invention provides methods, comprising: providing; a subject having endogenous anti-Gal antibody and an injured tissue; and a preparation comprising an α-gal epitope having a terminal α-galactosyl; and applying the preparation to the injured tissue to produce a treated injured tissue. In some embodiments, the terminal α-galactosyl is selected from the group consisting of Galα1-3Gal, Galα1-6Gal and Galα1-2Gal. In some preferred embodiments, the α-gal epitope is part of a molecule selected from the group consisting of a glycolipid (e.g., α-gal epitope linked to ceramide), a glycoprotein (e.g., α-gal albumin), proteoglycan and/or a glycopolymer (e.g., α-gal polyethylene glycol). In some particularly preferred embodiments, the glycolipid comprises α-gal nanoparticles. Also provided are methods in which the preparation further comprises anti-Gal antibodies bound to the α-gal nanoparticles. In some preferred embodiments, the preparation is part of an injured tissue care device selected from the group consisting of biodegradable material such as collagen, alginate or cellulose, gels, biological matrices, semi-permeable films, aerosol and foams. In some embodiments, the glycolipid preparation is derived from a source selected from the group consisting of rabbit red blood cells, bovine red blood cells, and other non-primate mammalian cells. Also provided are methods in which the glycolipid preparation further comprises an antibiotic. Moreover, in some particularly preferred embodiments, the applying comprises one of the groups consisting of injection into the injured tissue, suspension in isotonic solution, ointment, lotion, topical solution, decellularized tissue from an animal and biodegradable patch, hydrocolloid and hydro gel.

In some preferred embodiments, the applying is under conditions such that complement activation within or adjacent to the injured tissue is enhanced, in some embodiments, complement activation comprises production of C5a, C4a and/or C3a. In some preferred embodiments, the applying is under conditions such that one or more of the following take place: monocyte and macrophage recruitment within or adjacent to the injured tissue is enhanced; injured tissue repair and regeneration are accelerated as a result of local monocytes and macrophage secretions; and angiogenesis in the injured tissue is enhanced. In some embodiments, the subject is selected from the group consisting of a human, an ape, an Old World primate, and a bird.

It is contemplated that the recruited and activated macrophages by the method described in this invention will induce repair and regeneration of the injured tissue or of the biomaterial implant by rapid and effective recruitment of stem cells by the activated macrophages or by the function of some of the recruited macrophages as stem cells. In some embodiments, the present invention provides treatments for tissue repair and regeneration in normal subjects and in subjects having tissues or organs with impaired biological activity, including but not limited to ischemic myocardium, injured nerves, injured cartilage, injured skeletal muscle, injured smooth muscle, injured connective tissue, injured liver, injured endocrine glands injured eyes and ears, lung tissue damaged by injuries such as, but not limited to smoke inhalation or asbestosis, injured gastrointestinal tract and injured bone, and in subjects implanted with biomaterials comprised of decellularized tissues or organs, tissues fixed by crosslinking or semisolid fillers and collagen sheets, all containing α-gal nanoparticles which interact in situ with the natural anti-Gal antibody.

In some embodiment, macrophages recruited and activated by α-gal nanoparticles can support the viability and function of administered stem cells and of mature cells converted into stem cells by various methods such as but not limited to acid shock and introduction of various genes. When such stem cells are administered within a suspension also containing α-gal nanoparticles, macrophages are recruited activated by α-gal nanoparticles. The activated macrophages secrete cytokines/growth factors which facilitate the survival of the stem cells and of cells converted into stem cells for periods long enough to enable their effective differentiation into cells that regenerate the injured tissue.

In some embodiments, the glycolipid preparation is derived from a source selected from the group consisting of rabbit red blood cells, bovine red blood cells, and other mammalian cells. In some embodiment the glycolipids with α-gal epitopes comprise nanoparticles that may also comprise phospholipids. Such nanoparticles may also comprise cholesterol. Also provided are methods in which the glycolipid preparation further comprises an antibiotic. Moreover, in some particularly preferred embodiments, the applying comprises one of the group consisting of applying of α-gal glycolipids comprised nanoparticles by injection into the injured tissue, or by any other application method known to those skilled in the art. In some preferred embodiments, the applying is under conditions such that complement activation within or adjacent to the injured tissue is enhanced, in some embodiments, the complement activation comprises production of C5a and/or C4a and/or C3a. In some preferred embodiments, the applying is under conditions such that one or more of the following take place: monocyte and macrophage recruitment and activation within or adjacent to the injured tissue is enhanced; angiogenesis in the injured tissue is enhanced; recruitment of stem cells into the injured tissue by said macrophages is enhanced; resulting in enhanced healing, repair and regeneration of said injured tissue. The stem cells recruited by the macrophages are either from areas of uninjured tissue near the injury site or from other areas in the body. It is contemplated that the enhanced, repair and regeneration occurs since the stem cells recruited and activated by the macrophages migrating into the treated injury, take instructive cues from differentiated cells located nearby, from the extracellular matrix (ECM) and from the microenvironment, to differentiate into cells with biological activities similar to those comprising the tissue prior to injury. It is contemplated that the macrophages recruited and activated following anti-Gal/α-gal epitopes interaction can further recruit and activate stem cells residing within the injured site prior to formation of fibrosis tissue in that site, thereby restoring the biological activity of the tissue and decreasing, or completely preventing scar formation.

In some embodiments, the treated subject is selected from the group consisting of a human, an ape, an Old World primate, and a bird. In some embodiments, the injured tissue may be any tissue including, but not limited to: brain, skeletal muscle, smooth muscle, myocardium, lung, connective tissue, eyes, ears, limbs, pancreas and other endocrine glands, nerve, liver, gastrointestinal tissue and organs, bone, and cartilage. Furthermore, the present invention contemplates embodiments in which the nanonparticles comprise blood group A antigens and the subject has a B or O blood type. In other embodiments the nanoparticles comprise blood group B antigens and the subject has an A or O blood type. In additional embodiments the injured tissue nanoparticles comprise tetanus toxoid (TT) and the subject has anti-TT antibody. In additional embodiment, the present invention also provides nanoparticles comprising a preparation comprising an α-gal epitope having a terminal α-galactosyl. In some preferred embodiments, the terminal α-galactosyl is selected from the group consisting of Galα1-3Gal, Galα1-2Gal and Galα1-6Gal. In some particularly preferred embodiments, the α-gal epitope is part of a molecule selected from the group consisting of a glycolipid (e.g., α-gal epitope linked via a carbohydrate chain or any other linker to ceramide or to part of fatty acid), a glycoprotein (e.g., α-gal albumin) a glycopolymer (e.g., α-gal polyethylene glycol), a proteoglycan and to any hydrocarbon. In some embodiments, the glycolipid comprises α-gal nanoparticles. Also provided are devices in which the preparation further comprises anti-Gal antibodies bound to the α-gal liposomes or α-gal nanoparticles. In some preferred embodiments, the device is in the form of one of the group consisting of injectable suspension in an aqueous fluid, adhesive bands, gels, semi-permeable films, ointments, hydrocolloids gel, hydrogels, aerosol and foams. In some embodiments, the glycolipid preparation is derived from a source selected from the group consisting of rabbit red blood cells, bovine red blood cells, and other non-primate mammalian cells, in some embodiment the glycolipids are produced in vitro by chemical or biological synthesis, in some preferred embodiments, the glycolipid preparation further comprises an antibiotic.

In some embodiments the nanoparticles present the sugar rhamnose linked to any spacer and interact with anti-rhamnose antibody that is present in humans (Chen et al. ACS Chemical Biology 6:185, 2011). When applied to injuries or incorporated into biomaterials used as implants rhamnose nanoparticles interact with anti-rhamnose antibodies. This interaction results in activation of complement, generation of chemotactic complement cleavage peptides as C5a, C4a and C3a that induce rapid recruitment of macrophages. These recruited macrophages bind via their Fcγ receptors the Fc portion of the anti-rhamnose antibodies coating the nanoparticles and thus are activated to secrete cytokines/growth factors and induce repair and regeneration of the injured tissue and further induce recruitment of stem cells. These recruited stem cells receive cues from the microenvironment and from the ECM to differentiate into cells that restore the biological activity of the treated tissue, or of the implant containing the rhamnose presenting nanoparticles.

The mechanistic basis for recruitment and activation of macrophages by α-gal nanoparticles and the use of these nanoparticles in several clinical settings are described below as non-limiting examples for this invention. Similar recruitment and activation of macrophages for inducing repair and regeneration of internal injuries can be achieved by additional substances containing α-gal epitopes which interact with the natural anti-Gal antibody including, but not limited to glycolipids, glycoproteins, proteoglycans and/or glycopolymer such as α-gal polyethylene glycol.

### II. Anti-Gal Antibodies and α-Gal Epitopes

Anti-Gal is an abundant natural antibody in humans constituting ˜1% of all serum immunoglobulins as provided for in Galili et al., J. Exp. Med. 160, 1519 (1984), incorporated herein by reference. This antibody interacts specifically with the α-gal epitope (Galα1-3Galβ1-4GlcNAc-R or Galα1-3Galβ1-3GlcNAc-R) on glycolipids and glycoproteins as disclosed in Galili, Springer Semin. Immunopathol. 15, 155 (1993), incorporated in its entirety by reference.

Anti-Gal is produced throughout life as a result of antigenic stimulation by bacteria of the gastrointestinal tract as described in Galili et al., Infect. Immun. 56, 1730 (1988). The α-gal epitope is synthesized by the glycosylation enzyme α1,3-galactosyltransferase (α1,3GT) and expressed in very large amounts on the cells of non-primate mammals (e.g. mice, rats, rabbits, dogs, pigs, etc.), prosimians and in New World monkeys (monkeys of South America) as provided for in Galili et al., J. Biol. Chem. 263, 17755 (1988), incorporated herein by reference. The α1,3GT gene was inactivated in ancestral Old World primates as provided for in Galili and Swanson Proc. Natl. Acad. Sci. USA 88, 7401 (1991). Thus humans, apes, and Old World monkeys (monkeys of Asia and Africa) lack α-gal epitopes and produce high titer anti-Gal antibodies as provided for in Galili et al Proc. Natl. Acad. Sci. (USA) 84, 1369 (1987) and in Galili et al., J. Biol. Chem. 263, 17755 (1988), both incorporated herein by references. Anti-Gal antibodies bind in vivo to α-gal epitopes when administered to humans or Old World monkeys. This is particularly evident in the context of xenotransplantation, where the in vivo binding of anti-Gal to α-gal epitopes on transplanted pig heart or kidney is the main cause for the rapid rejection of such grafts in humans and Old World monkeys as disclosed in Galili., Immunol. Today 14, 480 (1993) and Collins et al., J. Immunol. 154, 5500 (1995), both of which are incorporated in their entirety by reference.

One of the main mechanisms mediating xenograft rejection is the activation of the complement cascade due to anti-Gal binding to α-gal epitopes on the endothelial cells of the xenograft. This results in the destruction of these endothelial cells by the activated complement molecules, causing collapse of the vascular bed and xenograft ischemia followed by its rapid rejection as provided for in Collins et al., J. Immunol. 154, 5500 (1995), hereby incorporated by reference. This in situ interaction of anti-Gal with newly introduced α-gal epitopes can be exploited for local activation of the complement system and recruitment of neutrophils, monocytes and macrophages into damaged tissues to accelerate the inflammatory response and the subsequent tissue repair. Due to its ubiquitous production in humans, anti-Gal is a superior choice for this purpose.

### III. The Natural Anti-Gal Antibody, α-Gal Epitopes and α-Gal Nanoparticles

The activity of anti-Gal can be manipulated in humans by the use of α-gal nanoparticles which are schematically represented in FIGS. 1 and 29. α-Gal liposomes and α-gal nanoparticles can be prepared from various materials and they are characterized by presenting multiple α-gal epitopes. In a non-limiting example, α-gal nanoparticles are composed of glycolipids with multiple α-gal epitopes (α-gal glycolipids), phospholipids and cholesterol (Wigglesworth et al J Immunol 186: 4422, 2011) (FIG. 2). Since α-gal glycolipids comprise most of the glycolipids in rabbit red blood cell (RBC) membranes and since these cell membranes are the richest source of α-gal glycolipids in mammals (Galili et al. supra 1987; Egge et al. J Biol Chem 260: 4927, 1985, Galili et al. J Immunol 178, 4676, 2007), rabbit RBC are a convenient natural source for preparation of α-gal nanoparticles (Wigglesworth et al. supra). For this purpose, glycolipids, phospholipids and cholesterol are extracted from rabbit RBC membranes in a mixture of chloroform and methanol (Galili et al. J. Immunol. 178:4676, 2007. The dried extract is sonicated in saline to generate liposomes with the size range of 0.1-100 μm comprised of α-gal glycolipids, phospholipids and cholesterol and which present multiple α-gal epitopes of the glycolipids in the extract. These liposomes (referred to in Wigglesworth et al. supra as α-gal liposomes) are further sonicated using a sonication probe into submicroscopic particles called α-gal nanoparticles which have the same composition as the α-gal liposomes, however their size range is 0.0001-0.5 μm. The α-gal nanoparticles suspension is further sterilized by filtration through a 0.2 μm filter (FIG. 30).

A schematic presentation of an α-gal nanoparticle is illustrated in FIG. 29. This nanoparticle has a wall of phospholipids and cholesterol in which α-gal glycolipids are anchored via the fatty acid tails of their ceramide portion. The illustrated glycolipid has 10 sugar units in its carbohydrate chain and 2 branches (antennae), each capped with an α-gal epitope. α-Gal glycolipids in rabbit RBC membranes are of various lengths ranging from 5 to 40 carbohydrate units carrying 1-8 branches each capped with an α-gal epitope (Galili et al. 2007 supra; Egge et al. 1985 supra; Hanfland et al. Carbohydrate Res 178:1, 1988; Honma et al. J Biochem (Tokyo) 90:1187, 1981). The various components of the α-gal nanoparticles are illustrated in FIG. 2A where the nanoparticles are dissolved in chloroform:methanol solution and run on a thin layer chromatography (TLC) plate. With the exception of the glycolipid ceramide tri-hexoside which lacks α-gal epitopes and which is present also in human RBC membranes, all other glycolipids with 5-25 carbohydrate units separated on the plate are capped with α-gal epitopes (i.e. are α-gal glycolipids) as indicated by immunostaining with a monoclonal anti-Gal antibody. The structure of these glycolipids with 5-25 carbohydrate units is illustrated in FIG. 2B.

Overall, the number of α-gal epitopes on α-gal nanoparticles is very high, corresponding to ˜10¹⁵ α-gal epitopes per mg α-gal nanoparticles (Wigglesworth et al. supra). From 1 liter of rabbit RBC it is possible to prepare 3-4 grams of α-gal nanoparticles. The α-nanoparticles are highly stable since they contain no tertiary structures. Accordingly, no changes in expression of α-gal epitopes were found in α-gal nanoparticles kept at 4° C. for 4 years in comparison with freshly produced α-gal nanoparticles.

The studies on anti-Gal mediated acceleration of injury regeneration by α-gal nanoparticles cannot be performed in standard experimental animal models since mice, rats, guinea-pigs, rabbits and pigs, all produce α-gal epitopes on their cells by the glycosylation enzyme α1,3galactosyltransferase (α1,3GT) and thus cannot produce the anti-Gal antibody (Galili et al 1987 supra; Galili et al. J Biol Chem 1988 supra). In addition to Old World monkeys, the only two nonprimate experimental animal models which are suitable for anti-Gal studies are α1,3GT knockout mice (KO mice) produced in the mid-1990s (Thall et al. J Biol Chem 270:21437, 1995; Tearle et al. Transplantation 61:13, 1996) and α1,3GT knockout pigs (KO pigs) produced in the last decade (Lai et al. Science 295:1089,2002; Phelps et al. Science 299:41, 2003). These two knockout animal models lack α-gal epitopes and can produce anti-Gal. Old World monkeys, which naturally produce the anti-Gal antibody can serve as animal models, as well.

### Anti-Gal/α-Gal Nanoparticles Interaction Induces Rapid and Extensive Macrophage Recruitment

Interaction between serum anti-Gal and α-gal epitopes on cells results in activation of the complement system. Transplantation of pig xenografts in monkeys is a demonstration of this complement activation. Binding of circulating anti-Gal antibody to the multiple α-gal epitopes on pig endothelial cells lining the blood vessels of pig kidney or heart xenografts, results in activation of the complement system that causes lysis of the endothelial cells, collapse of the vascular bed and hyperacute rejection of the xenograft within 30 minutes to several hours (Simon et al. Transplantation 56:346,1998; Xu et al. Transplantation 65:172, 1998). A similar activation of complement occurs when serum anti-Gal binds to the multiple α-gal epitopes on α-gal nanoparticles. This complement activation results in the generation of chemotactic complement cleavage peptides that are among the most potent physiologic chemotactic factors. These include C5a and C3a complement cleavage peptides which induce rapid chemotactic migration of macrophages into the site of α-gal nanoparticles application as schematically illustrated in FIG. 29) (Wigglesworth et al. supra).

In studies with α-gal nanoparticles injected intradermal in anti-Gal producing KO mice, macrophages were found to be recruited by this chemotactic mechanism. The neutrophils reached the injection site within 12 h and disappeared after 24 h whereas macrophages reached the injection site within 24 h and continued migrating into that site for several days (FIG. 31) (Wigglesworth et al. supra). The identity of the migrating cells as macrophages could be determined by immunostaining with the macrophage specific antibody (Wigglesworth et al. supra). The macrophages were found at the injection site for 14-17 days and completely disappeared without changing skin architecture within 21 days. No granulomas and no detrimental inflammatory responses were found in such α-gal nanoparticles injection sites.

The recruitment of macrophages by α-gal nanoparticles could be further validated in a large animal model of α1,3galactosyltransferase knockout (KO) pigs. These KO pigs produce the natural antibody as well as humans do (Galili Xenotransplantation 20:267, 2013) Topical application of α-gal nanoparticles on skin wounds of GT-KO pigs results in a much more extensive recruitment of macrophages into the treated wounds than control wounds treated with saline (FIG. 38). Following their recruitment monocytes/macrophages migrating into the injection site bind the anti-Gal coated (opsonized) α-gal liposomes via their Fcγ receptors (FcγR) as demonstrated in FIG. 30.

### IV. Binding of Anti-Gal Antibody by α-Gal Liposome

In addition to the effects of α-gal nanoparticles, manipulation of the anti-Gal antibody also could be demonstrated with α-gal liposomes (i.e. particles with the same structure as α-gal nanoparticles, but with a size that enables their identification in a microscope). Recruitment of neutrophils, monocytes and macrophages into sites of infection or tissue damage is directed by a concentration gradient of fragments of activated complement molecules such as C5a, C4a and C3a. Injection of molecules or particulate material bearing α-gal epitopes is contemplated to result in local interaction between endogenous anti-Gal antibodies and the exogenous α-gal epitopes, followed by activation of the complement system. One example of particulate material carrying multiple α-gal epitopes is α-gal liposomes, which can be prepared from chloroform:methanol extracts of rabbit red blood cell (RBC) membranes as shown in FIGS. 1 and 2. These liposomes are comprised of rabbit RBC glycolipids, phospholipids and cholesterol. Since most rabbit RBC glycolipids have α-gal epitopes, these liposomes carry many of these epitopes. When the α-gal liposomes are injected intradermally or into other tissues, a high local concentration of α-gal epitopes is generated, which is available for binding to anti-Gal antibodies. Both the anti-Gal antibody and complement are contemplated to reach the injection site due to local rupture of capillaries by the injecting needle. The activation of complement and generation of C5a, C4a and C3a fragments, following anti-Gal interaction with α-gal epitopes, results in a local inflammatory reaction that induces capillary dilation, and accumulation of serum proteins at the injection site (including more anti-Gal and complement proteins). This leads to further binding of anti-Gal to the injected α-gal liposomes and activation of complement, ultimately resulting in an amplification of the inflammatory process and the increased formation of chemotactic factors for recruitment of additional neutrophils, monocytes and macrophages into the injection site. Other liposomes that bear α-gal epitopes or other molecules carrying one or several α-gal epitopes are also suitable for enhancing the beneficial inflammatory response occurring at the injection site.

The monocytes/macrophages migrating into the injection site bind the anti-Gal coated (opsonized) α-gal liposomes via their Fcγ receptors (FcγR). The interaction of the Fc portion of anti-Gal (upon opsonization of α-gal liposomes) with FcγR on the monocyte and macrophage cell surface induces the activation of these cells, differentiation of the monocytes into macrophages and further activation of the macrophages to secrete a wide range of pro-healing cytokines/growth factors. Activated macrophages have been shown to secrete a variety of growth factors and cytokines including for instance: vascular endothelial cell growth factor (VGEF), epidermal growth factor (EGF), fibroblast growth factor (FGF), insulin-like growth factor (IGF), IL-1 and IL-6 as disclosed in DiPietro, Shock 4, 233-40 (1995); Rappolee and Werb, Curr. Topics Microbial Immunol. 181, 87-140 (1992) and Singer et al., New England Journal of Medicine 341, 738-46 (1999), all of which are hereby incorporated by reference.

The effect of α-gal liposomes on recruitment of macrophages and wound healing is localized to the injection site and has little to no systemic effect. The three components of the exemplary α-gal liposomes, α-gal glycolipids, phospholipids and cholesterol, are not immunogenic and therefore do not elicit a de novo immune response presumably because phospholipids and cholesterol are found in all mammalian species and because α-gal glycolipids in and of themselves do not activate T cells as disclosed in Tanemura et al., J. Clin. Invest. 105, 301 (2000). Accordingly, analysis of the antibody response to α-gal liposomes by ELISA (using α-gal liposomes as the solid phase antigen) revealed that antibody titers to α-gal liposomes were not elevated at 35 or 40 days post-injection. Moreover in experiments performed in anti-Gal seropositive mice, administration of α-gal liposomes did not cause abnormal behavior post-injection or increased morbidity or mortality.

Thus, injection of a preparation of α-gal liposomes in water, saline or other excipient into an infected wound, is contemplated to result in anti-Gal binding, activation of complement, generation chemotactic factors, rapid recruitment of neutrophils followed by monocytes and macrophages, phagocytosis of the infectious agent, debridement of the wound, and migration of regenerating cells into the wound. Secretion of epithelial growth factor by the activated macrophages results in epithelization, e.g. proliferation of epithelial cells to close the wound. The destruction of infectious agents and debridement of the wound by the inflammatory cell infiltrate and subsequent migration of fibroblasts and proliferation of epithelial cells is contemplated to accelerate wound healing and tissue repair.

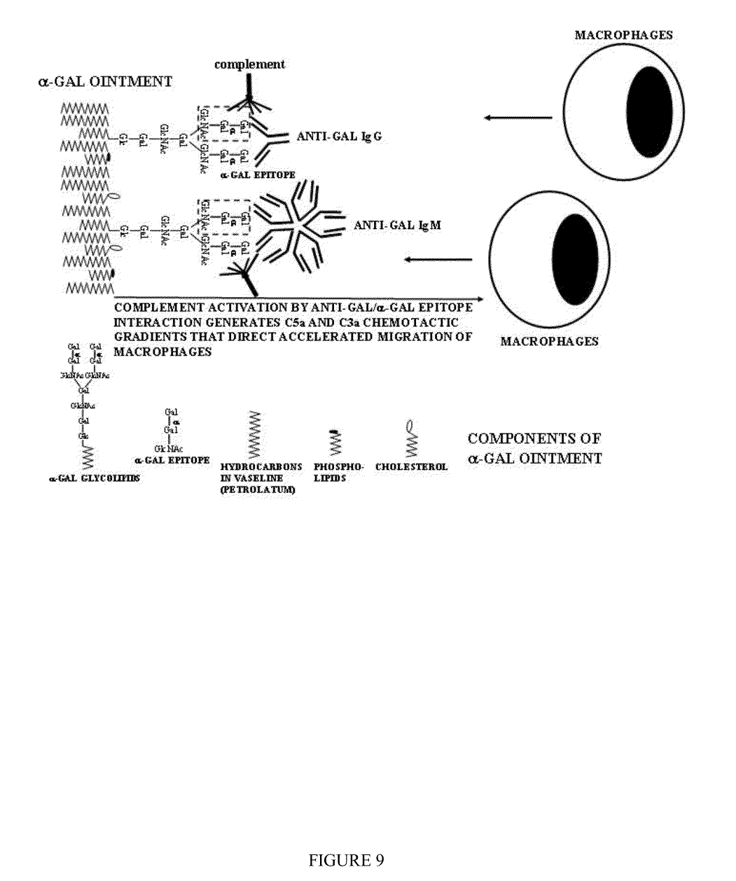

A similar accelerated healing of wounds can be achieved by topical application of α-gal ointment onto injured skin areas of various wounds including burns as shown in FIG. 9. The α-gal epitopes of α-gal glycolipids within this ointment bind the anti-Gal antibody, activate complement, generate complement degradation factors C5a and C3a due to cleavage of complement molecules, recruit granulocytes, monocytes and macrophages to the treated site and thus, induce accelerated healing of the injured area.

### V. Wound Healing Applications

The compositions and methods of the present invention are suitable for treating various wounds in normal subjects and in subjects having impaired healing capabilities, such as diabetics, heart disease and/or cardiac surgical subjects, and aged subjects.

In one embodiment, the present invention contemplates a method for inducing rapid recruitment and activation of macrophages by α-gal liposomes that interact with natural anti-Gal antibodies. In one embodiment, the α-gal liposomes accelerate wound healing while reducing scar formation. Although it is not necessary to understand the mechanism of an invention, it is believed that because humans naturally produce anti-Gal antibodies that constitute ˜1% of IgG, IgM and IgA immunoglobulins in their serum, topical application of α-gal liposomes may result in accelerated wound healing also in a clinical setting. Galili et al., 1984 “A unique natural human IgG antibody with anti-α-galactosyl specificity” J. Exp. Med. 160:1519-1531; Galili, U, 2005 “The α-gal epitope and the anti-Gal antibody in xenotransplantation and in cancer immunotherapy” Immunology and Cell Biology 83:674-686; Hamadeh et al. 1995 “Human secretions contain IgA, IgG and IgM anti-Gal (anti-α-galactosyl) antibodies” Clin. Diagnos. Lab. Immunol. 2:125-131; and Parker et al., 1994 “Characterization and affinity isolation of xenoreactive human natural antibodies” J Immunol. 153(8):3791-803.

Very high amounts of α-gal epitopes on glycolipids of α-gal liposomes may enhance their interaction with anti-Gal antibodies and induce a strong complement activation. Since there are ˜10¹⁵ α-gal epitopes/mg α-gal liposomes, topical application on wounds is believed to result in a high concentration of α-gal epitopes on a wound surface, thereby allowing for a robust local interaction with anti-Gal antibodies released from damaged capillaries and the ensuing local activation of the complement cascade.

Liposomes that do not express α-gal epitopes have been used in wound healing as vesicles for delivery of substances to wounds that affect wound healing such as superoxide dismutase, hemoglobin, or of genes that encode growth factors as demonstrated in studies of Vorauer-Uhl et al., 2002 “Reepithelialization of experimental scalds effected by topically applied superoxide dismutase: controlled animal studies” Wound Repair Regen. 10:366-371; Plock et al., 2009 “Hemoglobin vesicles improve wound healing and tissue survival in critically ischemic skin in mice” Am J Physiol Heart Circ Physiol. 297:H905-910; and Jeschke et al., 2007 “The combination of IGF-I and KGF cDNA improves dermal and epidermal regeneration by increased VEGF expression and neovascularization” Gene Ther. 14:1235-1242, respectively.

Although it is not necessary to understand the mechanism of an invention, it is believed that the α-gal liposomes deliver multiple α-gal glycolipids in their membranes, rather than within the liposomes, to mediate their therapeutic effects. The data presented herein show that α-gal liposome treatment, when compared to other wound healing treatments, has the distinct advantage of harnessing of at least two immunological mechanisms for accelerating the healing process: i) anti-Gal/α-gal liposome interaction activates complement to produce complement cleavage peptides that induces rapid extravasation of monocytes, conversion of the extravasating monocytes into macrophages, and chemotactic migration into the treated wound; and ii) Fc/FcγR interaction between anti-Gal coated α-gal liposomes and recruited macrophages results in activation these cells and secretion of cytokines that promote wound healing.

These data confirm that anti-Gal/α-gal liposome interaction activates complement using a complement consumption assay. For example, the effect of complement activation on macrophage recruitment was demonstrated in vivo in KO mice by inhibition of macrophage recruitment in the presence of cobra venom factor, a potent inhibitor of complement activation (FIG. 18). In addition to the generation of large activated macrophages, activation of these cells led to their production of cytokines (FIGS. 20 and 21). Increased expression of IL1α. IL6, Pdgfb, Fgf2, Csf1, Csf2, Tnf, and VEGF suggested that these genes may be involved in wound healing.

TNFα is considered to be a proinflammatory cytokine that is also involved in induction of early angiogenesis. Wang et al., 1999 “Macrophages are a significant source of type 1 cytokines during mycobacterial infection” J Clin Invest. 103(7):1023-1029; and Arras et al., 1998 “Monocyte activation in angiogenesis and collateral growth in the rabbit hindlimb” J Clin Invest. 101(1):40-50. Also observed was that anti-Gal coated α-gal liposomes activated macrophages to secrete VEGF in vitro (FIG. 15).

One of the earliest morphological events associated with the anti-Gal/α-gal liposome interaction is redness around an injection site (i.e., for example, at approximately 48 h post injection) (FIG. 19). Although it is not necessary to understand the mechanism of an invention, it is believed that this redness may reflect local angiogenesis due to secretion of VEGF from activated macrophages and/or vasodilation of capillaries and small blood vessels in the area of this antibody/antigen interaction. It is possible that the vasodilation is induced by complement cleavage peptides in a manner similar to the initiation of an immune mediated inflammatory response due to antibodies binding to invading bacteria.

In one embodiment, the present invention contemplates that rapid recruitment and/or activation of macrophages by anti-Gal/α-gal liposome immune complexes leads to accelerated wound healing. For example, by comparing the extent of epidermis regeneration in α-gal liposome treated wounds to that in control wounds, this treatment is believed to decrease the healing time by ˜50% (FIGS. 22-24). The data presented herein demonstrate histologic observations of macrophages wherein α-gal liposome treated wounds at 72 h was associated with regeneration of epidermis on day 6. One possible mechanism for this effect is that anti-Gal/α-gal liposome interaction may induce a rapid recruitment of the macrophages and the activation of these cells to produce cytokines that mediate wound healing. These observations are consistent with the skin burn healing data, also presented herein. The efficacy of the /α-gal liposomes to induce healing of wounds in KO mice was further increased when their size was decreased by sonication into /α-gal nanoparticles (FIG. 22)

Although it is not necessary to understand the mechanism of an invention, it is believed that an increased production of cytokines/growth factors by activated macrophages may promote healing in association with reduced hyperplasia in skin tissues and a decrease in scar formation in healing wounds. Formation of scar tissue, i.e. of a dense connective tissue lacking skin appendages, is a physiologic default mechanism for wound healing that occurs after the closure of the wound with regenerating epidermis. A rapid anti-Gal mediated activation of recruited macrophages may secrete cytokines that promote tissue healing thereby leading to a restoration of the cellular components of normal skin, prior to the initiation of the scar formation process. The histological data showing long term recovery (i.e., for example, day 28) strongly suggests that the α-gal liposome treatment does not induce hyperplasia and formation of scars in the skin tissues during the healing process (FIG. 26).

Activation of macrophages in wounds has been demonstrated by application of immunomodulating substances such as carrageenan and/or BCG as shown by Kelley et al., 1988 “Influence of hypercholesterolemia and cholesterol accumulation on rabbit carrageenan granuloma macrophage activation” Am J Pathol. 131:539-546; and Aguiar-Passeti et al., 1997 “Epithelioid cells from foreign-body granuloma selectively express the calcium-binding protein MRP-14, a novel down-regulatory molecule of macrophage activation” J Leukoc Biol. 62:852-858. However, these treatments also result in a non-beneficial prolonged inflammatory immune responses that may be manifested as a chronic granulomas. Subsequent to α-gal liposome treatment, however, no chronic granuloma formation was observed in wound healing, for example, by at least one month after initiation of α-gal liposome treatment. Although it is not necessary to understand the mechanism of an invention, it is believed that this implies that the rapid recruitment and activation of macrophages is not followed by any additional immune response to the treating substance. It is believed that α-gal liposomes lack immunogenicity because they do not contain any antigenic proteins capable of activating T cells. Tanemura et al., 2000 “Differential immune responses to α-gal epitopes on xenografts and allografts: implications for accommodation in xenotransplantation” J. Clin. Invest. 105:301-310. The data presented herein confirms that no antibody response was observed in α-gal liposome treated KO mice, whereas mice immunized with pig kidney membranes (PKM) (positive control) readily produced antibodies that bound to the α-gal liposomes. See, FIG. 28A. The PKM-induced antibodies are exclusively anti-Gal as indicated by their complete neutralization (i.e. lack of binding to the α-gal liposomes) by α-gal BSA (i.e. synthetic α-gal epitopes linked to BSA). Topical application of 10 mg α-gal liposomes on burns for 2 weeks also did not induce any antibody response. See, FIG. 28B. Since IgG response requires both T helper and B cells activation, these data imply that α-gal liposomes treatment does not elicit a new immune response.

The α-gal epitope itself, like other antigens comprised of carbohydrate chains of the complex type (e.g. blood group A and B antigens), does not activate T cells. In the absence of T cell help, the α-gal epitope also does not elicit a B cell immune response. Galili, U., 2004. “Immune response, accommodation and tolerance to transplantation carbohydrate antigens” Transplantation 78:1093-1098. Moreover, the interaction between FcγR on the recruited macrophages and anti-Gal coating the α-gal liposomes results in the rapid internalization of these liposomes due to effective phagocytosis and their elimination from the wound. Following the removal of liposomes, the recruited macrophages disappear within 3-4 weeks and do not elicit a chronic immune response or a granuloma within the treated wound.

Treatment with α-gal liposomes in the clinical setting is of potential significance. Decreasing the healing time of wounds will reduce morbidity as well as decrease the costs associated with acute and chronic wound treatment, which are expected to increase significantly in the coming years. Sen et al., 2009 “Human skin wounds: a major and snowballing threat to public health and the economy” Wound Repair Regen. 17:763.

Observations suggesting that the accelerated wound healing may be observed in human patients with wounds treated with α-gal liposomes include, but are not limited to: i) anti-Gal antibodies are present in very large amounts in all humans that are not severely immunocompromised; ii) anti-Gal antibodies in human serum effectively binds to α-gal liposomes and induces complement activation; iii) human anti-Gal antibodies immunocomplexed with α-gal epitopes readily binds to FcγR on macrophages; and iv) cultured human macrophages activated in vitro by hypotonic shock were found to accelerate wound healing in patients with deep sternal wounds and with ulcers. Orenstein et al., 2005 “Treatment of deep sternal wound infections post-open heart surgery by application of activated macrophage suspension” Wound Repair Regen. 13:237-242; and Danon et al., 1997 “Treatment of human ulcers by application of macrophages prepared from a blood unit” Exp Gerontol. 32:633-641.

Another advantage of administering α-gal liposomes on wound dressings is an ease of use when compared to injection of activated macrophages into wounds. Topical administrations do not require specialized equipment and facilities for in vitro culturing of macrophages. It is further possible that the treatment with α-gal liposomes in humans may be even more effective than the KO mice data described herein as complement activity in human serum is many fold higher than that in mouse serum. Galili et al., 2007 “Intratumoral injection of α-gal glycolipids induces xenograft-like destruction and conversion of lesions into endogenous vaccines” J. Immunol. 178:4676-4687. Anti-Gal is believed to be present in all individuals who are not severely immunocompromized, including diabetic patients and elderly individuals. Galili et al., 1995 “Increased anti-Gal activity in diabetic patients transplanted with fetal porcine islet cell clusters” Transplantation 59:1549-1556; and Wang et al., 1995 “Variations in activity of the human natural anti-Gal antibody in young and elderly populations” J. Gerontol. (Med. Sci.) 50A: M227-M233. Consequently, the effective recruitment and activation of macrophages by α-gal liposomes may “jumpstart” the healing process in chronic wounds of diabetic patients and elderly individuals who display impaired wound healing.

α-gal liposomes are believed to be highly stable and their α-gal epitopes do not alter their structure during prolonged storage. α-gal epitopes, in contrast to biologically active proteins, have no folding or tertiary structures leading to their robust stability. Furthermore, α-gal epitopes do not undergo oxidation for prolonged periods and can be stored for years without losing activity. This conclusion can be inferred from studies on blood group antigens. The structure of the α-gal epitope is very similar to that of blood group A and B antigens. Galili et al., 1985 “Human natural anti-α-galactosyl IgG. II. The specific recognition of α (1-3)-linked galactose residues” J. Exp. Med. 162: 573-582; Galili et al., 2002 “Anti-Gal A/B, a novel anti-blood group antibody identified in recipients of ABO incompatible kidney allografts” Transplantation 74:1574-1580. Because of their stability, these blood group antigens can be detected in >2000 yr old Egyptian mummies. Crainic et al., 1989 “ABO tissue antigens of Egyptian mummies” Forensic Sci Int. 43:113-124. Thus, if α-gal liposomes are found to be effective in accelerating wound healing in humans, they can be stored for prolonged periods and delivered to wounds in many forms including sprays, hydrogels, on wound dressings, in suspension, or incorporated into devices and dressings that are currently used for treating injuries.

Repair and regeneration of internal injured tissues has been suggested to be dependent on effective local recruitment and activation of macrophages. Duffield et al., 2005 “Selective depletion of macrophages reveals distinct, opposing roles during liver injury and repair” J Clin Invest. 115:56-65. Therefore, it is possible that delivery of α-gal liposomes to such injuries (e.g. internal ischemic tissue and trauma injuries) may result in the accelerated repair and regeneration of the biological activity of the injured tissue, while avoiding irreversible scar formation.

A. Surgical Incisions

In a preferred embodiment, compositions comprising α-gal liposomes are used to enhance internal and external wound healing in surgical incision sites that have been damaged as a result of ischemia. Injection of α-gal liposomes into the area surrounding the sutures and ischemic tissue enhances recruitment of neutrophils, monocytes and macrophages into the surgical incision site ultimately resulting in improved wound healing. In this way, the present invention is suitable for shortening the time required for healing of wounds and repair of damaged tissues following surgery. A specific non-limiting example is the removal of a colon carcinoma and reconnection of the colon wall at the site of tumor resection.

B. Cardiac Tissue

In another embodiment, compositions comprising α-gal liposomes are used to treat injured muscle tissue. While not limiting the scope of the invention in any way, one example contemplated by the invention is the treatment of skeletal muscle damaged due to physical trauma or heart muscle damaged due to ischemia. Injection of α-gal liposomes into the injured or damaged muscle tissue enhances recruitment of neutrophils, monocytes and macrophages into the injured muscle ultimately resulting in improved tissue repair. In particular the inflammatory cell infiltrate recruits stem cells or myoblasts, which subsequently differentiate into functional cardiac myocytes in treated heart muscle, or fuse and differentiate into functional skeletal muscle fibers in treated skeletal muscle. In this way the biomechanical function of the damaged muscle is restored.

In preferred embodiments, the compositions and methods of the present invention are used to promote healing due to cardiac tissue damage in both normal subjects and in subjects having impaired healing capabilities. For example, the heart is comprised of myocardium tissue. This tissue may be damaged or otherwise compromised during cardiac trauma, disease or related events including but not limited to cardiac surgery, coronary heart disease, cardiomyopathy, cardiovascular disease, ischemic heart disease, myocardial infarction, heart failure, hypertensive heart disease, inflammatory heart disease and valvular heart disease. Mortality rates for cardiac surgical procedures continue to be a cause for concern. For example, repairs of congenital heart defects are currently estimated to have 4-6% mortality rates. One non-limiting example of wounds that may receive benefit from the compositions and methods of the present invention are infected deep sternum incisions that are observed in an appreciable number of open-heart surgery patients. Injection of α-gal glycolipid preparations (e.g., α-gal liposomes) into the infected area of the sternum enhances recruitment of neutrophils, monocytes and macrophages into the surgical incision site ultimately resulting in faster and improved wound healing.

In another embodiment α-gal liposomes are injected into cardiac muscle injured by ischemia. These injected α-gal liposomes bind in situ the endogenous natural anti-Gal antibody. This antigen/antibody interaction of α-gal liposomes/anti-Gal results in local activation of the complement system and generation of the chemotactic cleavage complement peptides C5a, C4a and C3a. These chemotactic factors direct the migration of macrophages into the injection site. Some of the macrophages have the potential of becoming stem cells. The macrophages further bind the anti-Gal coated α-gal liposomes via their Fcγ receptors (FcγR) are activated by this interaction. This activation results in the secretion of a variety of cytokines/growth factors. In addition, the activated macrophages induce local angiogenesis and generate a microenvironment that may be conducive to the recruitment of stem cells from adjacent uninjured myocardium or from other sites in the body. Stem cells recruited by the α-gal liposomes treatment receive instructive cues from uninjured cardiomyocytes, from the cytoskeleton of the heart muscle and from the microenvironment within the heart muscle and develop into cardiomyocytes that repopulate the injured myocardium. Ultimately the treatment with α-gal liposomes injected into the injured heart muscle can result in repair and regeneration of the heart muscle and restoration of the heart muscle function.

C. Nerve Tissue

Administration of α-gal liposomes into nerves damaged by physical or other trauma, or because of nerve degeneration, enhances recruitment of neutrophils, monocytes and macrophages. The activated macrophages debride the damaged nerve tissue and secrete VEGF that induces angiogenesis at the nerve injury site. This angiogenesis is required for effective axonal sprouting in order to bridge the neural lesion area. The axonal sprouting occurs along new capillaries growing at the lesion site. The VEGF secretion by macrophages that are activated by α-gal liposomes greatly increases the formation of new capillaries. Dray C et al. Qunatitative analysis by in vivo imaging of the dynamics of vaxcular and axonal networks in injured mouse spinal cord. Proc. Natl. Acad. Sci. (USA) 106:9459-64. The sprouting axons grow along the new capillaries into the post lesion axonal tube, thereby regenerate the nerve. Thus, the treatment with α-gal liposomes can induce axonal regeneration and restoration of nerve pulse conductivity via the regenerating nerve. This is contemplated to result in partial or complete restoration of function of the treated nerve. In the absence of such a treatment, the fibrosis of the nerve lesion site, as a default repair mechanism, may occur in a large proportion of patients with nerve injury and result in an irreversible damage to the injured nerve.

In one embodiment, the present invention contemplates compositions and methods to recruit stem cells, for healing and/or repairing damaged or injured brain tissue. In one embodiment, α-gal liposomes are injected intracranially into injured brain areas. In one embodiment, the brain is a human brain. In one embodiment, the brain injury comprises damage including, but not limited, that following ischemic infarction. In one embodiment, the α-gal liposomes are injected at any volume that is suitable for injection into the injured brain tissue and at a concentration ranging between 0.001 and 500 mg/ml. Although it is not necessary to understand the mechanism of an invention, it is believed that the interaction between the injected α-gal liposomes and the anti-Gal antibody activates complement and the generated chemotactic complement cleavage peptides recruit monocytes and macrophages to the injection site. The macrophages are activated by Fc/FcγR interaction with anti-Gal coated α-gal liposomes and secrete cytokines and growth factors that promote healing of the injured brain tissue and recruit stem cells. These stem cells proliferate and differentiate in to brain cells that repair and regenerate the injured brain tissue.

D. Burns

In further embodiments, compositions comprising α-gal glycolipids and/or α-gal epitopes are applied to skin burns. Their interaction with the anti-Gal antibody, leaking to the burn surface together with other serum proteins, results in complement activation recruitment of neutrophils, monocytes and macrophages and ultimately resulting in accelerated healing of the burn.

The data presented herein was collected in accordance with Example 26. Normal mouse skin displays an epidermis comprised of 2-3 layers of epithelial cells, the underlying dermis stained pink, and the hypodermis containing multiple fat cells. See, FIG. 27A. As in second degree burns in humans, the thermal injury in mouse skin destroys both epidermis and dermis, whereas damage to the hypodermis is minimal. See, FIG. 27B. No significant differences between experimental (α-gal liposomes) and control (saline) treatments are observed 1 day after injury (not shown).

On Day 3, the number of neutrophils migrating into the hypodermis of burns treated with α-gal liposomes was several fold higher than in control burns. See, FIGS. 27C and 27D. Mononuclear cells with morphology of macrophages are detected only in the α-gal liposomes treated burns.

On Day 6, α-gal liposomes treated burns display extensive regeneration of epidermis as 50-100% re-epithelialization of the surface areas with newly formed epidermis. See, FIG. 27F. However, epidermis regeneration in control burns is only marginal on Day 6. See, FIG. 27E. Recruited neutrophils in α-gal liposomes treated burns are found on Day 6 on the surface of the regenerating epidermis, whereas many mononuclear cells with macrophage morphology are detected in the dermis. See, FIG. 27F. In contrast, neutrophils are found mostly within the outer region of the injured dermis in saline treated burns and the number of macrophages is relatively low. See, FIG. 27E. In both treatments, the dermis region is filled with eosinophilic material which may reflect local secretion of collagen.

The accelerated healing of the α-gal liposomes treated burns, is dose dependent. Treatment for 6 days with bandages coated with 1.0 mg instead of 10 mg α-gal liposomes results on average in 23% regeneration of the epidermis instead of 70% observed with the higher dose. No significant differences are observed in healing of burns treated with 0.1 mg α-liposomes and healing of control burns treated with saline.

On Day 9, regenerating epidermis covers on average 25% of burn surface in control burns and 85% of surface area in α-gal liposomes treated burns. By Day 12, epidermis regeneration is complete in both groups. See, Table 1.

TABLE 1
Summary of histological characteristics
in burns treated with α-gal liposomes

| | | | | ^{b}% of |
|---|---|---|---|---|
| Days post | Treatment of | ^{a}number of | ^{a}number of | epidermis |
| treatment | burn | neutroph [symbol] | macrop [symbol] | regeneration |
| ^{c}3 | Saline control | 132 + 18 | 0 | 0 |
| | α-gal liposomes | 520 + 87 | 41 + 27 | 7 + 2.8 |
| ^{c}6 | Saline control | 173 + 47 | 24 + 16 | 6 + 4.1 |
| | α-gal liposomes | Neutrophils | 84 + 35 | 70 + 23 |
| | | above th [symbol] | | |
| | | epidermis | | |
| ^{c}9 | Saline control | 235 + 95 | 37 + 12 | 25 + 11 |
| | α-gal liposomes | No visible | 105 + 10 | 85 + 14 |
| | | neutrophi [symbol] | | |
| ^{c}12 | Saline control | No visible | 21 + 5.3 | 100 |
| | | neutroph [symbol] | | |
| | α-gal liposomes | No visible | 96 + 26 | 100 |
| | | neutroph [symbol] | | |
| ^{d}3 in WT^{e} | Saline control | 124 + 26 | 17 + 7.1 | 0 |
| mouse | α-gal liposomes | 134 + 48 | 21 + 9.6 | 0 |
| ^{d}6 in WT | Saline control | 141 + 33 | 39 + 16 | 0 |
| mouse | α-gal liposomes | 153 + 64 | 37 + 13 | 8 + 7.3 |

^{a}Number of infiltrating cells was determined in histological sections by counting cells within a rectangular area marked in the microscope lens at magnification of x400. The rectangle with a size corresponding to 100 × 200 μm was placed to include both dermis and hypodermis. Neutrophils were identified by segmented nuclei and macrophages were tentatively identified by the kidney or oval shaped nuclei and large size of the cells.
^{b}% of epidermis regeneration was determined histologically by the proportion of the burn surface covered with the newly formed epidermis.
^{c}Mean + Standard Deviation from 5 mice per group;
^{d}Mean + Standard Deviation from 4 mice per group.
^{e}WT = Wild Type [symbol]
indicates data missing or illegible when filed

E. Diabetes

In an additional embodiment, the disclosed α-gal liposome can be combined in compositions with at least one anti-Gal antibody. The mixture of these antigen and antibody will result in increased recruitment of neutrophils, monocytes and macrophages to the injured area. Such treatment is ideal, for example, for aged individuals or subjects with advanced diabetes patients where poor vascularization prevents sufficient anti-Gal antibody from reaching injured areas. Alternatively, such treatment may be applicable to non-primate mammals lacking the anti-Gal antibody. The applied immune complexes activate complement and thus accelerate wound healing.

In some embodiments, the local anti-Gal mediated activation of complement and subsequent recruitment of activated macrophages into an injection site is achieved by employing a variety of natural or synthetic macromolecules carrying multiple α-gal epitopes. Various commercially available glycolipids (Dextra Laboratories, Ltd., United Kingdom) are suitable for use in the compositions and methods of the present invention for generation of α-gal liposomes. These glycolipids include but are not limited to: i) Galα-3Gal glycolipids: α1-3 galactobiose (G203); linear B-2 trisaccharide (GN334); and Galili pentasaccharide (L537). Various other glycoconjugates with α-gal epitopes available from Dextra include for instance: Galα1-3Galβ1-4Glc-BSA (NGP0330); Galα1-3Galβ1-4(3-deoxyGlcNAc)-HAS (NGP2335); Galα1-3Galβ1-4GlcNAcβ1-HDPE (NGL0334); and Galα1-3Gal-BSA (NGP0203).

Several non-limiting examples of additional macromolecules with α-gal epitopes that are suitable for injection and subsequent in situ binding to anti-Gal antibodies and local activation of complement include: mouse laminin with 50-70 α-gal epitopes as disclosed in Galili, Springer Seminars in Immunopathology 15, 155 (1993), incorporated herein by reference; multiple synthetic α-gal epitopes linked to BSA as disclosed in Stone et al., Transplantation 83, 201 (2007), hereby incorporated by reference; GAS914 produced commercially by Novartis and disclosed in Zhong et al., Transplantation 75, 10 (2003), incorporated herein by reference; the α-gal polyethylene glycol conjugate TPC as disclosed in Schirmer et al., Xenotransplantation 11, 436 (2004), hereby incorporated by reference, and α-gal epitope-mimicking peptides linked to a macromolecule backbone as disclosed in Sandrin et al., Glycoconj. J. 14, 97 (1997), hereby incorporated by reference. Injection or topical application of such macromolecules results in local interaction with the pre-formed anti-Gal antibody present in all humans, activation of complement, recruitment of inflammatory cells into the injection site and differentiation of these cells thereby effecting improvements in the duration and quality of wound healing.

In still further embodiments a glycoprotein carrier such as the human alpha1-acid glycoprotein (α1-AG) is utilized. α1-AG is abundant in human serum, non-immunogenic in humans, and can be obtained commercially in purified form. α1-AG is a small glycoprotein (e.g., 40 kDa) with five N-linked carbohydrate chains, each with 2 or more antennae with the terminal structure sialic acid-Galβ1-4GlcNAc-R as disclosed in Schmid et al., Biochemistry 13, 2694-2697 (1973), incorporated herein by reference. To synthesize the α-gal epitopes on the α1-AG the sialic acid is first removed to expose the penultimate N-acetyllactosamine (Galβ1-4GlcNAc-R). Next the appropriate carbohydrate is added to this backbone to synthesize the α-gal epitope. Briefly, neuraminidase is used to remove the terminal sialic acid, followed by the addition of an α1-3Gal unit using a galactosyltransferase (e.g., recombinant α1,3 galactosyltransferase) and uridine diphosphate-galactose as the sugar donor as disclosed in Galili, Cancer Immunol. Immunother. 53, 935-945 (2004), hereby incorporated by reference.

F. Osteoarthritis (OA)

In preferred embodiments, the methods and compositions of the present invention are used to reduce the symptoms associated with osteoarthritis (OA), a disease that may also be referred to as degenerative arthritis. Traditionally, treatment for osteoarthritis is limited to pain relievers including but not limited to non-steroidal anti-inflammatory drugs (NSAIDS), corticosteroids, COX-2 selective inhibitors and topical creams. In more severe cases of OA the subject receives either injections of local anesthetics such as lidocaine or undergoes joint replacement surgery for the affected area. In a further embodiment, injection of α-gal liposomes into the synovial cavity, or into damaged cartilage within injured bones enhances recruitment of neutrophils, monocytes and macrophages into the synovial cavity or cartilage ultimately resulting in tissue repair. In particular, macrophages activated by the binding of α-gal liposome/anti-Gal antibody complexes mediate debridement of the damaged cartilage and through secretion of growth factors and cytokines direct migration of chondroblasts into the damaged cartilage. The chondroblasts in turn secrete collagen and other cartilage matrix proteins and glycosaminoglycans (GAG), resulting in repair and remodeling of the damaged articular or meniscus cartilage within the treated joint. Similarly, macrophages activated by the binding of α-gal liposome/anti-Gal antibody complexes mediate debridement of the damaged bone and through secretion of growth factors and cytokines recruit osteoclasts and osteoblasts into the injection site for repair and remodeling of the damaged bone.

G. Diabetes

In preferred embodiments, the present invention is used to promote healing in tissue damage as a result of diabetes in both normal subjects and in subjects having impaired healing capabilities. Diabetes can cause many complications, including but in no way limited to acute complications such as hypoglycemia, ketoacidosis, or non-ketotic hyperosmolar coma, long-term complications including but not limited to cardiovascular disease, chronic renal failure, retinal damage, blindness, nerve damage and microvascular damage. Poor healing of many superficial wounds due to diabetes can lead to many diseases including but not limited to gangrene, which may require amputation. In the developed world, diabetes is the most significant cause of adult blindness in the non-elderly and the leading cause of non-traumatic amputation in adults, and diabetic nephropathy is the main illness requiring renal dialysis in the United States. The α-gal liposomes of the present invention may be preferably used in wound care devices in patients with diabetes, in order to induce effective wound healing by local activation of complement as a result of anti-Gal antibody binding to α-gal liposomes. In still another embodiment, the invention relates to the use of α-gal liposomes in wound care devices applied to a wound in a subject following either diabetic complications or the natural progression of the disease.

In another embodiment, α-gal liposomes are used for injection into the pancreas in diabetic patients, in order to restore the formation of Langerhans Islets in the pancreas. These islets contain cells that secrete insulin. Injection of α-gal liposomes at a concentration ranging between 0.001 and 500 mg/ml. The injection is performed by ultrasound endoscopy, or by laparoscopy, or any other type of injection, into the pancreas tissue of diabetic patients induces the recruitment and activation of macrophages that promote tissue repair. Some of these macrophages have stem cell potential and can differentiate into Langerhans Islet cells. In addition, the activated macrophages secrete cytokines and growth factors that promote recruitment of stem cells which give rise within the pancreas to formation of Langerhans Islets, which secrete several hormones and include, but are not limited to, insulin.

For example, in some patients with Type I diabetes and in some of the patients with Type II diabetes the Langerhans Islets have been destroyed. In one embodiment, the present invention contemplates restoring biologically active Langerhans Islets in the pancreas of these patients. It is believed that such restoration would thereby provide endogenous insulin and cure the state of diabetes. In one embodiment, α-gal liposomes at a concentration ranging between 0.001 and 500 mg/ml are injected into the pancreas by a device enabling endoscopy ultrasound, or by laparoscopy, or by any other procedure which enables for direct injection of the α-gal liposomes into the pancreas. The interaction between the injected α-gal liposomes and the anti-Gal antibody activates complement and the generated chemotactic complement cleavage peptides recruit monocytes and macrophages to the injection site. The macrophages are activated by Fc/FcγR interaction with anti-Gal coated α-gal liposomes and secrete cytokines and growth factors that recruit stem cells. These stem cells and/or stem cells originating from macrophages proliferate and differentiate into Langerhans Islet cells that form the islets and secrete endogenous insulin.

H. Nerve System

In some embodiments, the methods and compositions of the present invention are used to restore structure and/or function to injured tissues of the central and peripheral nerve system. In one embodiment, the present invention contemplates treating brain tissue that is injured as a result of conditions including, but not limited to, ischemia (i.e., for example, infarct), or trauma. Although it is not necessary to understand the mechanism of an invention, it is believed that injection of α-gal liposomes into the injured brain tissue promotes recruitment and activation of macrophages which transdifferentiate into neurons and/or recruit and activate stem cells. It is also believed that the activated macrophages secrete cytokines and growth factors that may promote repair of the injured brain tissue. α-gal liposomes recruits and induces stem cell migration from adjacent uninjured brain tissue, or from other site in the body, or are the result of transdifferentiation from macrophages to the injured brain tissue. These stem cells are believed to differentiate into brain cells that replace the injured tissue, based on cues from normal brain cells, matrix and microenvironment. Ultimately, α-gal liposomes injection into the injured brain tissue restores partially or completely the structure and function of the treated tissue.

In another embodiment, administration of α-gal liposomes into nerves damaged by physical trauma, or by other types of trauma, or because of nerve degeneration, enhances recruitment of neutrophils, monocytes and macrophages into the injured area of the nerve and activates the recruited macrophages. The activated macrophages debride the damaged nerve tissue and secrete nerve growth factors that recruit stem cells and induce axonal regeneration. The regeneration is mediated by VEGF that is secreted by the activated macrophages. VEGF induces angiogenesis of capillaries. The newly formed capillaries further induce effective axonal sprouting. The axonal sprouts grow along the capillaries into the post lesion axonal tube and regenerate the nerve. This regeneration restores nerve pulse conductivity via. This is contemplated to result in partial or complete restoration of function of the treated nerve.

I. Musculoskeletal

In preferred embodiments the methods and compositions of the present invention are used to restore structure and function of injured parts of the musculoskeletal system. In one embodiment the present invention can be used to treat skeletal muscle injured due to physical trauma or to ischemia. Injection of α-gal liposomes into the injured muscle tissue enhances recruitment of neutrophils, monocytes and macrophages into the injured muscle. The recruited macrophages are activated by Fc/FcγR interaction with anti-Gal coated α-gal liposomes and secrete cytokines and growth factors that promote repair of the injured muscle tissue, by recruiting stem cells. A proportion of the macrophages also has the potential of stem cells. The stem cells recruited by macrophages or originating from macrophages differentiate into myoblasts that fuse into myotubes which repair the injured muscle and restore its biological activity.

In a further embodiment, injection of α-gal liposomes into the synovial cavity, or into damaged cartilage in joints enhances recruitment of neutrophils, monocytes and macrophages into the synovial cavity or cartilage ultimately resulting in tissue repair. In particular, macrophages activated by the binding of α-gal liposome/anti-Gal antibody complexes mediate debridement of the damaged cartilage and through secretion of growth factors and cytokines direct migration of stem cells that differentiate into chondroblasts within the damaged cartilage. The chondroblasts in turn secrete collagen and other cartilage matrix proteins, glycosaminoglycans and proteoglycans, resulting in repair and regeneration of the damaged articular or meniscus cartilage within the treated joint. Similarly, macrophages activated by the binding of α-gal liposome/anti-Gal antibody complexes mediate debridement of the damaged bone and through secretion of growth factors and cytokines recruit osteoclasts and osteoblasts into the injection site for repair and regeneration of the damaged bone.

J. Vascular System

In some embodiments, the present invention contemplates compositions and methods for the recruitment of stem cells, resulting in repair and regeneration of the blood vessel wall. For example, α-gal liposomes may be administered to patients with damaged blood vessels or having an anastomoses. In one embodiment, the injured blood vessel may be surrounded by a wound care device containing α-gal liposomes at a concentration ranging between 0.001 and 500 mg/ml. This device can be in the form of a gel, plasma clot or fibrin clot surrounding part or the whole injured blood vessel. Alternatively, a collagen sheet or any biodegradable or non-biodegradable sheet containing the α-gal liposomes or having on its surface α-gal liposomes and which can be shaped into a tube around the injured blood vessel can be used to apply α-gal liposomes around the injured blood vessel. Although it is not necessary to understand the mechanism of an invention, it is believed that the interaction between the injected α-gal liposomes and the anti-Gal antibody activates complement and the generated chemotactic complement cleavage peptides recruit monocytes and macrophages to the injection site. The macrophages are activated by Fc/FcγR interaction with anti-Gal coated α-gal liposomes and secrete cytokines and growth factors that promote the repair of the injured blood vessel wall. These secreted cytokines and growth factors also recruit stem cells that proliferate and differentiate into cells that enable the regeneration of the intact blood vessel wall. Some of the recruited macrophages, which have stem cell potential, also may trans-differentiate into cells that repair the injured blood vessel.

K. Gastrointestinal System

In one embodiment, the present invention contemplates compositions and methods for recruiting stem cells, resulting in repair and regeneration of the gastrointestinal wall. In one embodiment, the patient comprises an ulcer and/or other injuries to the gastrointestinal tract. The treatment methods described herein are applicable to any damage to the wall at any part of the gastrointestinal tract. In one embodiment, an injured gastrointestinal area may be injected with α-gal liposomes at a concentration ranging between 0.001 and 500 mg/ml. Although it is not necessary to understand the mechanism of an invention, it is believed that the interaction between the injected α-gal liposomes and the anti-Gal antibody activates complement and the generated chemotactic complement cleavage peptides recruit monocytes and macrophages to the injection site. The macrophages are activated by Fc/FcγR interaction with anti-Gal coated α-gal liposomes and secrete cytokines and growth factors that recruit stem cells and promote the repair of the injured tissue. The recruited stem cells proliferate and differentiate into cells that replace the injured cells and repair the damaged gastrointestinal wall at the injection site.

L. Epidermal Wound Healing

1. In Vitro Interaction of Anti-Gal Coated α-Gal Liposomes with KO Mouse Macrophages Induces VEGF Secretion

α-gal liposomes were generated using rabbit red blood cells (RBC) that provide multiple glycolipids with α-gal epitopes (i.e., for example, α-gal glycolipids). As previously shown, incubation of rabbit RBC membranes with chloroform and methanol results in extraction of phospholipids, cholesterol and multiple α-gal glycolipids. Galili et al., 2007 “Intratumoral injection of α-gal glycolipids induces xenograft-like destruction and conversion of lesions into endogenous vaccines” J. Immunol. 178:4676-4687. Rabbit RBC have the highest concentration of α-gal glycolipids among mammals, ranging in size from 5 to 40 carbohydrates, and having one, two or multiple branches, each capped with an α-gal epitope. Sonication in saline of the dried organic extract from rabbit RBC membranes results in formation of liposomes constructed of a membrane of phospholipids and cholesterol and multiple α-gal glycolipids anchored in that membrane. Because of the multitude of α-gal epitopes on these liposomes (i.e., for example, ˜10¹⁵ α-gal epitopes/mg liposomes), they have been designated α-gal liposomes and were found to interact effectively with anti-Gal produced by KO mice. Abdel-Motal et al., 2009 “Mechanism for increased immunogenicity of vaccines that form in vivo immune complexes with the natural anti-Gal antibody” Vaccine 27:3072-3082; and Galili et al., 2010 “Accelerated healing of skin burns by anti-Gal/α-gal liposomes interaction” Burns 36:239-251.

The ability of anti-Gal/α-gal liposomes to interact with and activate KO mouse macrophages was determined. Such activation was documented by measuring VEGF secretion. For these experiments, α-gal liposomes pre-coated with KO mouse anti-Gal antibodies were incubated with KO mouse peritoneal macrophages. Non-antibody coated α-gal liposomes incubated with macrophages served as controls. Liposome binding to the macrophages was determined by flow cytometry following double staining of fluorescein (green) coupled Bandeiraea simplicifolia IB4 lectin (BS lectin-binding specifically to α-gal epitopes and rhodamine (red) coupled anti-CD11b Ab (specific for macrophages). Galili et al., 1985 “Human natural anti-α-galactosyl IgG. II. The specific recognition of α(1-3)-linked galactose residues” J. Exp. Med. 162:573-82; and Galili et al., 1987 “Evolutionary relationship between the anti-Gal antibody and the Galα1-3Gal epitope in primates” Proc. Natl. Acad. Sci. (USA) 84:1369-1373. Non-antibody coated α-gal liposomes adhered to ˜15% of the KO mouse macrophages. See, FIG. 15A. However, binding increased by ˜4-fold when the liposomes were coated with anti-Gal antibody (55%). See, FIG. 15B. These data are similar to those observed using a system in which tumor cells coated with mouse or human anti-Gal antibodies bound to macrophages via Fc/FcγR interaction. LaTemple et al., 1999 “Increased immunogenicity of tumor vaccines complexed with anti-Gal: Studies in knockout mice for α1,3galactosyltranferase” Cancer Res. 59:3417-3423; and Manches et al., 2005 “Anti-Gal mediated targeting of human B lymphoma cells to antigen-presenting cells: a potential method for immunotherapy with autologous tumor cells” Haematologica 90:625-634.

VEGF secretion was quantified in peritoneal macrophage cultures co-cultured with anti-Gal coated or non-antibody coated α-gal liposomes to determine whether macrophages were activated by the anti-Gal/α-gal liposome complexes. Macrophages co-cultured with anti-Gal coated α-gal liposomes produced 2-4 fold more VEGF than the same macrophages incubated with α-gal liposomes lacking anti-Gal antibody. See, FIG. 15C. The latter macrophages produced low levels of VEGF similar to that secreted by macrophages cultured in the absence of liposomes. These findings support the assumption that interaction between the Fc portion of anti-Gal antibody bound to α-gal liposomes and FcγR on macrophages activates these cells to produce and secrete tissue healing cytokines/growth factors.

2. Recruitment of Macrophages into Skin Sites Injected with α-Gal Liposomes

Studies on the in vivo interactions between anti-Gal antibodies with α-gal liposomes require animal models that lack self expressed α-gal epitopes and can generate anti-Gal antibodies. A mouse model that lacks α-gal epitopes is the α1,3galactosyltransferase (α1,3GT) knockout mouse (KO mice). Thall et al., 1995 “Oocyte Gal α1,3Gal epitopes implicated in sperm adhesion to the zona pellucida glycoprotein ZP3 are not required for fertilization in the mouse” J. Biol. Chem. 270:21437-21440. These mice produce anti-Gal antibodies in titers comparable to those attained in humans following immunization with pig kidney membranes (PKM). Anti-Gal produced in the immunized mice displays characteristics (i.e., for example, classes and subclasses) similar to human anti-Gal antibodies. Abdel-Motal et al., 2006 “Increased immunogenicity of HIV gp120 engineered to express α-gal epitopes” J. Virol. 80:6943-6951.

The effect of anti-Gal/α-gal liposome interaction on localized recruitment of macrophages was studied in vivo in anti-Gal antibody producing KO mice injected subcutaneously with 10 mg α-gal liposomes in 0.1 ml saline. Control liposomes were generated using KO pig RBC that lack α-gal epitopes due to targeted disruption of the α1,3GT gene in these knockout pigs. Byrne et al., 2008 “Proteomic identification of non-Gal antibody targets after pig-to-primate cardiac xenotransplantation” Xenotransplantation 15:268-276. As with KO mice, KO pigs completely lack α-gal glycolipids, therefore liposomes produced from their RBC membranes completely lack α-gal epitopes. Because KO pig liposomes lack α-gal epitope they do not bind IgG antibodies in KO mouse serum, as indicated in ELISA in wells coated with KO pig liposomes. See, FIG. 16. The marginal binding of IgG antibodies at the lowest dilutions is likely to be nonspecific binding of serum IgG to the ELISA wells. In contrast, anti-Gal in these sera readily binds to α-gal liposomes coating ELISA wells. This binding is detectable (>1.0 O.D.) even at serum dilutions of 1:160, whereas no such binding was observed at the lowest dilution (1:20) in wells coated with KO pig liposomes. Previous studies by flow cytometry demonstrated a similar interaction of anti-Gal IgG and IgM antibodies with α-gal liposomes incubated in KO mouse serum. Galili et al., 2010 “Accelerated healing of skin burns by anti-Gal/α-gal liposomes interaction” Burns 36:239-251.

Skin specimens were obtained from euthanized mice at various time points after subcutaneous injection of 10 mg α-gal liposomes were fixed and stained with hematoxylin and eosin (H&E). Injection sites where liposomes were dissolved by ethanol and removed during the staining process are visualized “empty” areas. See, FIGS. 17A, 17C, 17F, and 17G. The data demonstrate that within 12 h, the injection site in the hypodermis was surrounded by neutrophils. See, FIGS. 17A and 17D. Most of the neutrophils disappeared by 24 h and were subsequently replaced by infiltrating macrophages. See, FIG. 17B, 17E, and FIG. 18. These cells were confirmed to be macrophages using F4/80 antibody, which specifically binds to macrophages. See, FIG. 17K. Macrophage recruitment appears to be highly dependent on activation of the complement cascade, as low macrophage recruitment was observed 24 h after co-injection of α-gal liposomes and cobra venom factor (CVF-20 μg), which inhibits complement activation. See, FIG. 17C and FIG. 18. The significance of α-gal epitope expression on liposomes for induction of rapid recruitment of macrophages through complement activation is further strengthened by failure of 10 mg KO pig liposomes to induce recruitment within 24 h post injection. See, FIG. 17F and FIG. 18.

Inspection of α-gal liposome injection sites after 4 and 6 days revealed a gradual increase in the size of macrophages and the formation of large clusters of these cells with almost no intercellular space. See, FIGS. 17K and 17J, respectively. Individual macrophages inspected after 6 days were very large (20-30 μm) and contained multiple vacuoles that represented the internalized α-gal liposomes. See, FIG. 17L. This morphology of infiltrating macrophages was observed up to 14 days post injection. See, FIG. 17H. However, by 3-4 weeks, all macrophages have disappeared and the injected skin displayed normal histology. See, FIG. 17I and FIG. 18. Parallel studies in mice injected subcutaneously with saline did not show evidence of recruitment of cells into the injection site at any time point (data not shown).

Subcutaneous injection of anti-Gal/α-gal liposomes resulted in changes in gross morphology shortly after the injection, as viewed from the hypodermis side of the skin. Two days post injection, redness was observed around the site of α-gal liposome injection, whereas no such redness was observed in injection sites of KO pig liposomes. See, FIG. 19. Subcutaneous injection of saline resulted in no induction of local redness (data not shown). Although it is not necessary to understand the mechanism of an invention, it is believed that the redness observed following α-gal liposome injection may be associated with local vasodilation of capillaries induced by complement cleavage products generated following anti-Gal/α-gal liposome interaction. It is also believed that some of the “redness” was due to angiogenesis and sprouting of new capillaries as a result of local secretion of VEGF by activated macrophages.

3. In Vivo Induction of Cytokine Gene Expression by Injected α-Gal Liposomes

The above in vitro studies showing that macrophages that interact with α-gal liposomes suggested that these cells might be activated following the Fc/FcγR interaction with anti-Gal antibody coating these liposomes. Macrophages migrating into an injection site of α-gal liposomes were assayed to determine if genes encoding cytokines that promote wound healing were being activated. To test this, KO mice were injected subcutaneously with 10 mg α-gal liposomes or with saline as control. After 48 h, the skin at the injection site was harvested, RNA extracted, mRNA was isolated and cDNA was synthesized and subjected to quantitative real time PCR (q-RT-PCR) with primers specific for 11 cytokine genes known to be produced by activated macrophages. GADPH was used as a control housekeeping gene for normalizing the cDNA. Gene expression in α-gal liposome injected KO mouse skin was calculated and expressed as relative fold change in comparison to saline injected skin specimens normalized to GAPDH expression.

Although there was a mouse to mouse variation, in the five skin specimens tested, six of the assayed genes: Il1a, IL6, Pdgfb, Fgf2 Csf1 and Csf2 displayed >3 fold increase in expression compared to controls. Activation of these wound healing promoting cytokines genes was observed in mice injected with α-gal liposomes. See, FIG. 20. These data coincide with the observed extensive recruitment of macrophages into the injection site. See, FIG. 18. These data strongly suggest that these activated genes are expressed in macrophages recruited by anti-Gal/α-gal liposome interaction.

In vivo activation of macrophage cytokine genes was further studied in a relatively pure macrophage population interacting with α-gal liposomes. Macrophages were recruited to the peritoneal cavity of KO mice 5 days post i.p. injection of thioglycolate. These mice were then injected i.p. with 30 mg α-gal liposomes. Control mice were injected with saline instead of α-gal liposomes (3 mice/group). After 24 h, the macrophages were harvested, their RNA extracted and subjected to q-RT-PCR to test for cytokine gene expression. Similar to the observations in activated skin macrophages, peritoneal macrophages activated by anti-Gal/α-gal liposome immune complexes increased expression of the Csf1 and Csf2 genes. See, FIG. 21. However, the most activated gene in all 3 mice, 24 h post injection, was the Tnf gene that displayed 9-11 fold amplification. Since this gene did not display a significant increased expression in skin macrophages assayed at 48 h post injection, these findings suggest that expression of cytokine genes may be altered in various time points.

4. α-Gal Liposome Treatment Accelerates Epidermal Healing of Skin Wounds

The above observations on accelerated recruitment of macrophages and activation of these cells following ant-Gal/α-gal liposome interaction suggested that topical application of these liposomes on wounds might induce accelerated healing. To test this, anesthetized KO mice were wounded by forming excisional skin wounds (˜3×6 mm oval excision) in which epidermis, dermis and upper part of the hypodermis were removed from the abdominal flank. The wounds were treated with 10 mg α-gal liposomes, KO pig liposomes (lacking α-gal epitopes), or saline on a 10×10 mm pad of spot bandages used as wound dressing. The gross appearance of the wound was documented on various days and the wound area removed from euthanized mice and subjected to histological analysis. Wound healing was determined by the percent of wound surface covered by regenerating epidermis.

The data was evaluated by histological analysis and by gross appearance on day 6 in specific cohorts of mice. Control wounds treated for 3 days with bandages that had saline displayed no evidence of regeneration of the epidermis and no significant infiltration of macrophages into the wound. See, FIGS. 22 and 23A, respectively. In contrast, wounds treated with α-gal liposomes for 3 days displayed extensive infiltration of mononuclear cells with macrophage morphology and which form a characteristic granulation tissue. See, FIG. 23B. These α-gal liposome treated wounds also exhibited a distinct initiation of epidermis regeneration, as indicated by the multilayered large epidermal cells observed over the newly formed dermis at border of the injured area. See, FIG. 22 and FIG. 23B. The regenerating epidermis covered on day 3, on average, 11% of the wound, whereas control wounds treated with KO pig liposomes or with saline displayed only a residual epidermis regeneration. See, FIG. 22.

By day 6, control saline treated wounds displayed extensive infiltration of macrophages into the regenerating dermis and initial regeneration of the epidermis. See, FIGS. 23C and 23E, respectively. However, at this time point, the regeneration of the epidermis is observed only at the periphery of the wound, whereas at the center of the wound, the dermis remains exposed. See, FIG. 23C. The leading edge of the regenerating epidermis on day 6, in saline treated wounds is shown. See, FIG. 23E.

In wounds treated for 6 days with saline or with KO pig liposomes, the regenerating epidermis covers only ˜20% of the wound surface. In contrast, the extent of epidermis regeneration in wounds treated with α-gal liposomes was much higher on day 6 and reached an average of ˜60% of the wound surface. See, FIG. 22 and FIG. 23. Further, on day 6 ˜35% of mice treated with α-gal liposomes displayed complete closure of the wound by regenerating epidermis. See, FIG. 23 and FIG. 24. In the other 65% of the mice, the regenerating epidermis covered ˜30-80% of the wound. The data show that α-gal liposome regenerated epidermis is thicker than normal epidermis (4-8 layers of epithelial cells vs. 2 layers, respectively), suggesting a highly proliferative state of epidermal cells. See, FIGS. 23D and 23F. Also, in many α-gal liposome treated wounds examined on day 6 the dermis was thicker than that in saline treated wounds, suggesting accelerated regeneration of the dermis. Compare, FIG. 23D with 23C. By day 9, only ˜40% of the surface of saline or KO pig liposome treated wounds was covered by regenerating epidermis. In comparison, most of α-gal liposome treated wounds showed complete epidermal closure with some displaying between approximately 60-90% regeneration. See, FIG. 22.

After 12 days of treatment, on average, ˜60% of wound surface in saline and KO pig liposome treated wounds were covered by the regenerating epidermis whereas most of the α-gal liposome treated wounds were completely covered by epidermis. Overall, the regeneration of epidermis in wounds treated with α-gal liposomes is approximately twice as fast as the physiologic regeneration of saline treated wounds. The similar rate of epidermis regeneration between saline treated and KO pig liposome treated wounds strongly suggests that accelerated regeneration in these studies is dependent on α-gal epitope presentation on the liposomes. Decreasing the size of the α-gal liposomes by further sonication resulted in α-gal liposomes with a submicroscopic size, referred to as α-gal nanoparticles. These α-gal liposomes with submicroscopic size (i.e. α-gal nanoparticles) display a higher efficacy of wound healing induction than α-gal liposomes of microscopic size. Thus, a complete closure of wounds treated with α-gal nanoparticles, indicated by 100% epidermis regeneration was observed already on day 6. See FIG. 22.

5. Regeneration of Dermis in Wounds as Evaluated by Trichrome Staining

Evaluation of connective tissue (i.e. dermis and hypodermis) regeneration in the wound can be performed following Trichrome staining. Trichrome stains the collagen fibers of the connective tissue within the dermis and hypodermis blue whereas epidermal and dermal residing cells are stained purple. α-gal liposomes treated wounds display regenerating dermis within 3 days post wounding. See, FIG. 25B. In contrast, no evidence for regeneration is observed in the saline treated wounds. See, FIG. 25A. Connective tissue of the dermis and hypodermis of the control wound is loose likely due to the lack of regeneration and/or fluid accumulation following injury. In the α-gal liposome treated wounds, initiation of dermal recovery is evidenced by the collagen fibers appearing beneath the regenerating epidermis. See, FIG. 25B. The uninjured dermis surrounding the wound is characterized by blue staining of collagen that is much denser than the newly formed collagen in the regenerating dermis. The presented histology suggests that collagen secreting fibroblasts are among the first cells recruited within 72 h post injury into wounds treated with α-gal liposomes.

Day 6, control wounds show newly formed dermis that contains multiple macrophages. See, FIGS. 25C and 25E. A distinct border between the newly formed dermis and the uninjured dermis in day 6 control wounds is present. Newly formed dermis filled with many cells is also observed in wounds treated with α-gal liposomes on day 6. See, FIGS. 25D and 25F. Some of the cells residing in the regenerating dermis are fibroblasts depositing collagen. Many of the other cells forming the granulation tissue in this dermis are likely macrophages that have been recruited into the wound.

6. α-Gal Liposome Treatment Reduces Scar Formation

Accelerated healing of wounds by α-gal liposomes could result in hyperplasia of the epidermis and/or scar formation in the dermis. To investigate this, wounds were treated for one month with saline or an α-gal liposome dressing (5 mice/group). Control wounds treated for one month with saline coated bandages displayed wide areas of dense dermis devoid of skin appendages, characteristic of scar formation. In addition, the regenerating epidermis in these control wounds is thicker than normal epidermis and has >5 layers of cells. See, FIGS. 26A-D. This scar formation is the physiologic default mechanism for filling the injured area with dense connective tissue and with epidermis that is thicker than in uninjured skin.

In contrast, epidermis in α-gal liposome treated wounds group displayed normal thickness of 2 cell layers and the density of collagen in the dermis based on Trichrome staining is normal. See, FIGS. 26E-H. Much of the healed wounds treated with α-gal liposomes also contained regenerating skin appendages such as hair follicles and sebaceous glands. It is notable that wounds treated with α-gal liposomes or with saline contain no granulomas at one month and that most macrophages have disappeared from the wound site. These observations imply that the rapid recruitment of stem cells into the wound and regeneration of the wound occurs faster than the onset of the fibrosis leading to scar formation. Thus, wounds treated with α-gal liposomes avoid scar formation by restoring normal structure of the skin prior to the formation of scar tissue.

### VI. Wound Care Devices

In some embodiments, the invention relates to the use of α-gal liposomes in wound care devices for aged subjects in order to induce effective wound healing by local activation of complement as a result of anti-Gal antibody binding to α-gal liposomes. In still another embodiment, the invention relates to the use of α-gal liposomes in wound care devices applied to a wound in a subject following trauma. While not limiting the scope of the present invention, one example of a use for the present invention is the treatment of a subject recovering from a car accident resulting in injuries to said subject.

In one embodiment, a wound care device comprises an injury care device selected from the group consisting of syringes, adhesive bands, compression bandages, sponges, gels, semi-permeable films, plasma clots, fibrin clots. In one embodiment, the device comprises physiological compositions including, but not limited to, solutions, suspensions, emulsions, creams, ointments, aerosol sprays, collagen containing substances, stabilizers, drops, matrix-forming substances, foams and/or dried preparation.

### VII. Pharmaceutical Compositions

The present invention further contemplates pharmaceutical compositions capable of: i) delivering α-gal epitopes; or ii) administering compositions that interact with α-gal epitopes. The pharmaceutical compositions of the present invention may be administered in a number of ways depending upon whether local or systemic treatment is desired and upon the area to be treated. Administration may be topical, pulmonary (e.g., by inhalation or insufflation of powders or aerosols, including by nebulizer; intratracheal, intranasal, epidermal and transdermal), oral, and/or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the oligonucleotide is administered in maintenance doses, ranging from 0.01 μg to 100 g per kg of body weight, once or more daily, to once every 20 years.

### VIII. Activation of Macrophages by Anti-Gal Coated α-Gal Nanoparticles

After the recruited macrophages reach the α-gal nanoparticles, the Fc portion of anti-Gal coating α-gal nanoparticles binds to Fcγ receptors (FcγR) on these macrophages (FIG. 29). This extensive binding to FcγR on macrophages is demonstrated in FIG. 30 where anti-Gal coated α-gal nanoparticles were incubated in vitro with cultured macrophages of α1,3GT knockout pig origin (KO pig). Multiple α-gal nanoparticles attach to the macrophages via the Fc/FcγR interaction. In the absence of anti-Gal, no significant binding of α-gal nanoparticles to macrophages was observed. This Fc/FcγR interaction generates a trans-membrane signal that activates the macrophages to produce various cytokines and growth factors (referred to as cytokines/growth factors) that promote tissue repair and regeneration. Analysis of growth factors secretion into the culture medium demonstrated increased production of vascular endothelial growth factor (VEGF), whereas quantitative PCR demonstrated increased production of fibroblast growth factor (FGF), interleukin 1 (IL1), platelet derived growth factor (PDGF) and colony stimulating factor (CSF). See FIG. 15C and FIG. 20 respectively). The extensive angiogenesis as an outcome of VEGF secretion by α-gal nanoparticles activated macrophages is demonstrated in vivo within wounds of KO pigs (FIG. 38). The density of blood vessels is much higher in the dermis of KO pig wounds treated with α-gal nanoparticles in comparison to wounds in the same pig that are treated with saline (FIG. 38). This increased blood vessels production is in accord with the increased production of VEGF by recruited macrophages that are activated following Fc/Fcγ receptor interaction with anti-Gal coated nanoparticles.

The present invention teaches the recruitment and activation of macrophages by α-gal nanoparticles in a variety of internal injuries. The invention also teaches the possible exploitation of this macrophage recruitment and activation for further recruitment of stem cells in order to induce repair and regeneration of injured and/or damaged tissues.

### IX. Macrophages Activated by Anti-Gal Coated α-Gal Nanoparticles Recruit Stem Cells

Among the cytokines/growth factors secreted by the activated macrophages there are those that induce recruitment of stem cells into the area of interaction with the α-gal nanoparticles. Stem cells have at least three characteristics: 1. Stem cells can proliferate and for colonies in vitro, 2. Some stem cells are pluripotent/pluripotential and can differentiate into various types of mature cells, and 3. When in contact with a distinct extracellular matrix (ECM), stem cells differentiate into cells that comprise the tissue of that ECM. According to these characteristics it is possible to demonstrate the recruitment of stem cells within biologically inert polyvinyl alcohol (PVA) sponge discs (10 mm diameter and 3 mm thickness) which contain α-gal nanoparticles and are implanted subcutaneously in KO mice producing anti-Gal. Cells which are recruited into PVA sponge discs containing α-gal nanoparticles, included in addition to the migrating macrophages, also cells that display an extensive ability to proliferate (200-500 cells per colony formed from one cell within a period of 5 days) (FIG. 14). The frequency of these colony forming cells among cultured macrophages from PVA sponges is calculated to be 3-5 cells/10⁵ macrophages. This is a similar frequency as that of mesenchimal stem cells in the bone marrow (Eisenberg et al. Stem Cells. 24:1236, 2006). This ability to proliferate (i.e. self-renew) and form colonies fulfils the first characteristics of stem cells listed above. These findings indicate that the macrophages recruited by anti-Gal/α-gal nanoparticles interaction, are capable of further recruitment of stem cells into the site of α-gal nanoparticle administration.

The pluripotent/pluripotential characteristics of the recruited stem cells is illustrated in FIG. 34 where the stem cells, recruited into subcutaneously implanted PVA sponge discs containing α-gal nanoparticles, differentiated within 5 weeks into nerves containing multiple axons (FIG. 32A) or into myotubes of striated skeletal muscle (FIG. 32B). In addition, connective tissue and blood vessels formation observed in FIG. 32 further support the notion that pluripotent/pluripotential stem cells are recruited by the macrophages that were recruited and activated as a result of anti-Gal binding to the a α-gal nanoparticles introduced into the inspected site.

The third characteristics of stem cells is their ability to be instructed to differentiate into mature cells as a result of interaction with the ECM of a given tissue. This characteristics was demonstrated with PVA sponge discs containing a suspension of 1 mg/ml α-gal nanoparticles mixed with 10 mg/ml homogenate of pig meniscus cartilage fragments devoid of α-gal epitopes. These PVA sponge discs were implanted subcutaneously in KO mice, retrieved after five weeks, fixed, sectioned and stained with hematoxylin and eosin (H&E) or with trichrome (for staining collagen in blue) (FIG. 33). A section through a full size of the PVA sponge disc is shown at a low magnification in FIG. 33A. The areas within the rectangles are areas of fibrocartilage formation. These areas of fibrocartilage growth are stained in red by H&E (FIG. 33B), and in deep blue by trichrome staining of the collagen fibers of the fibrocartilage (FIGS. 33C and 33D). In PVA sponge discs containing meniscus fibrocartilage fragments but no α-gal nanoparticles (FIG. 33E), the formation of fibrocartilage is not observed. The newly formed fibrocartilage has a similar structure as the fibrocartilage in the meniscus (FIG. 33F), however the collagen fibers and fibrocondrocytes do not have the complete parallel orientation due to space constraints within the spaces of the PVA sponge discs. The de-novo formation of fibrocartilage demonstrated in FIG. 33 implies that the α-gal nanoparticles within the sponge discs recruited macrophages which were activated and secreted cytokines/growth factors that recruited stem cells, which, in turn, were directed by the fragmented meniscus cartilage ECM to differentiate into fibrochondroblasts that produce collagen characteristic to the meniscus cartilage.

It is therefore contemplated that α-gal nanoparticles within implanted decellularized tissues or organs that undergo tissue engineering processing will similarly recruit macrophages. These recruited macrophages will be activated by the Fc/Fcγ receptor interaction with anti-Gal coating the α-gal nanoparticles., These activated macrophages will secrete cytokines/growth factors which recruit stem cells into these tissue engineered implants. The recruited stem cells will be guided by the extracellular matrix (ECM) within the implant to differentiate into cells that regenerate the structure of the implanted tissue/organ to that prior to the decellularization process and will further restore the biological activity of the implanted tissue/organ.

X. Treatment of Injured Tissues by Administration of α-Gal Nanoparticles into the Injury Site

The present invention teaches a method for treatment of internal injuries by injection of α-gal nanoparticles into internal injuries for the purpose of recruitment of macrophages into the injury site. Alternatively, application of α-gal nanoparticles to injury sites may be performed by the use of semi-solid gels, plasma clot, fibrin glue, and other synthetic or natural biomaterials prepared to contain α-gal nanoparticles. In addition, inhalation of aerosolized suspension of α-gal nanoparticles will result in administration of α-gal nanoparticles into the alveoli and airways of damaged lungs. The interaction of anti-Gal within the treated patient with the injected α-gal nanoparticles will activate the complement system and generate complement cleavage chemotactic peptides. These peptides will induce rapid and extensive migration of macrophages into the site of the administered α-gal nanoparticles within the injured or damaged tissue. The macrophages recruited by the α-gal nanoparticles will further interact via their Fcγ receptor with the Fc portion of anti-Gal coating the α-gal nanoparticles as illustrated in FIG. 29. This interaction will activate the macrophage to secrete a variety of cytokines/growth factors which mediate angiogenesis and repair of the injured tissue. Among the secreted cytokines/growth factors are those that recruit stem cells into the injury site. The recruited stem cells origin may be from the mesenchimal stem cells, stem cells from the uninjured adjacent tissue or from any other source in the body. It is contemplated that the invention described here for rapid recruitment and activation of macrophages by α-gal nanoparticles will ultimately induce repair and regeneration of the treated injury prior to the occurrence of the fibrosis process. This fibrosis process is the default mechanism for healing of injured tissues in the body. Without the regenerative effect of α-gal nanoparticles the fibrosis process results in the irreversible formation of a scar. Once the scar is formed, it prevents the regeneration of the original tissue structure and function.

The submicroscopic α-gal nanoparticles may be prepared by sonication of α-gal liposomes into submicroscopic particles which can be sterilized by filtration through a filter that removes bacteria and protozoa as well as by other standard sterilization methods known to those skilled in the art. In one non-limiting example the filter can be with pores of 0.2 μm in size. The α-gal nanoparticles can be prepared also from any type of nanoparticles known to those skilled in the art and linking α-gal epitopes to these nanoparticles by a variety of chemical, biochemical and/or enzymatic methods known to those skilled in the art. The amount of α-gal nanoparticles which should be administered into injury sites may vary from 1.0 nanogram to 100 grams/kg body weight and preferably should be within the dose range of 1 mg and 100 mg.

In one embodiment α-gal nanoparticles may be injected into ischemic myocardium in order to induce rapid and extensive migration of macrophages into the injured tissue and the activation of the recruited macrophages within the ischemic myocardium. In post myocardial infarction, macrophages migrate to the injured myocardium, debride it of dead cells and secrete cytokines/growth factors that have various pro-healing effects, including, but not limited to angiogenesis and recruitment of stem cells. The recruited stem cells receive cues from the adjacent healthy cells, the microenvironment and the extracellular matrix (ECM) to differentiate into cardiomyocytes that regenerate the tissue and restore its physiologic activity (Minatoguchi et al. Circulation 109:2572,2004; Dewald et al. Circ. Res. 96:881,2005; Yano et al. J Am Coll Cardiol 47:626,2006; Strauer et al. Circulation 106:1913, 2002). Myocardium with limited ischemic damage may display spontaneous regeneration by this mechanism. However in more extensive ischemic damage, migration of macrophages into injured myocardium and the recruitment of stem cells are processes that are too slow to prevent irreversible fibrosis which is the default mechanism for tissue repair. It is contemplated that this fibrosis may be reduced and possibly prevented by direct transendocardial injection or transpericardial injection of α-gal nanoparticles into the injured cardiac muscle by an injecting catheter of syringe, shortly after the ischemia event, so that regeneration is enabled and fibrosis is prevented. The injection of α-gal nanoparticles into the ischemic myocardium may be performed also by any other method known to those skilled in the art. Injected α-gal nanoparticles will bind the anti-Gal antibody and induce rapid chemotactic migration of macrophages as illustrated in FIG. 34 and FIG. 35. The migrating macrophages recruited into the injection area within the ischemic myocardium will be activated by Fc/Fcγ receptor interaction with the Fc portion of anti-Gal on the nanoparticles, migrate throughout the ischemic myocardium and further recruit stem cells from the circulation or from uninjured adjacent myocardium. It is contemplated that, similar to the stem cell differentiation presented in FIG. 33, the recruited stem cells may be guided by the microenvironment and by the ECM to differentiate into cardiomyocytes that repopulate the ischemic myocardium and restore its biological activity.

In another embodiment administration of α-gal nanoparticles will induce recruitment and activation of macrophages into nerve injury sites and thus may induce regeneration of nerves that are severed or damaged. Activated macrophages are pivotal in regeneration of injured nerves, as in spinal cord injury or in other nerve injuries in the body. Regeneration of nerves requires regrowth of multiple sprouts from the injured axons. These sprouts attempt to reconnect across the lesion and grow into the distal axonal tube of the damaged neurons. This axonal sprout growth depends on cytokines/growth factors such as VEGF secreted by macrophages migrating to the injury site and inducing local angiogenesis, since the axonal sprouts grow along newly formed capillaries within the nerve lesion site (Dray et al. Proc Natl Acad Sci USA 106:9459, 2009). If this growth of axonal sprouts is delayed because of insufficient recruitment and/or activation of macrophages in the injury site, the ongoing fibrosis will irreversibly prevent regeneration of the injured nerve. The α-gal nanoparticles applied to nerve injury site will bind anti-Gal and induce rapid macrophage migration and activation for the local secretion of VEGF in a manner similar to the recruitment and activation of macrophages presented in FIGS. 31, 34, 35 and 38. This process results in local angiogenesis similar to that presented in FIG. 38 and growth of many axonal sprouts which increase the probability of axonal growth into the distal portion of the axonal tubes, ultimately inducing regeneration of the injured nerve. The application of α-gal nanoparticles to induce nerve regeneration may performed by various methods known to those skilled in the art, including, but not limited to the use of a conduit in which the nanoparticles are mixed with a semi-solid filler such as keratin hydrogel-filled conduit (Horton, and Auguste Biomaterials 33:6313, 2012) or conduits containing nerve tissue ECM (Liu et al. Biomaterials 30:3865, 2009), or by using hydrogels, plasma clot, fibrin glue, suspension, aerosol, or any other type of α-gal nanoparticles suspension suitable for application to injured nerves. An example of a plasma clot containing α-gal nanoparticles as a semi-solid filler for application of α-gal nanoparticles is illustrated in FIG. 36.

In yet another embodiment, α-gal nanoparticles may be applied to injured skeletal muscles or injured smooth muscles for the rapid recruitment and local activation of macrophages. A non-limiting example is the treatment of damaged skeletal muscle due to physical trauma.

Injection of α-gal nanoparticles into the injured or damaged muscle tissue induces accelerated recruitment of macrophages into the injured muscle (FIG. 13) and activation of these macrophages by the interaction between the Fc portion of the anti-Gal antibody coating the α-gal nanoparticles and Fcγ receptors on the macrophages. It is contemplated that these activated macrophages secrete cytokines/growth factors that recruit stem cells, precursor cells and/or myoblasts, which subsequently differentiate into functional myocytes that fuse into myotubes that comprise functional skeletal muscle fibers in treated skeletal muscle. An example of skeletal muscle developing in a site where α-gal nanoparticles are introduced within a biologically inert sponge made of polyvinyl alcohol (PVA) and implanted subcutaneously in an anti-Gal producing KO mouse is illustrated in FIG. 32.

In a further embodiment, application of α-gal nanoparticles into the synovial cavity, or into defects of damaged cartilage induces rapid and extensive recruitment of macrophages into these sites of damage and activation of these macrophages by the interaction between the Fc portion of the anti-Gal antibody coating the α-gal nanoparticles and Fcγ receptors on the macrophages. The activated macrophages will secrete cytokines/growth factors that recruit stem cells which will differentiate into chondroblasts. These chondroblasts, in turn, secrete collagen and other cartilage matrix proteins and glycosaminoglycans, resulting in repair and remodeling of the damaged cartilage. It is further contemplated that a mixture of α-gal nanoparticles and a homogenate consisting of fragmented cartilage in the form of paste made of any filler known to those skilled in the art, or without a filler, is to be applied to defects of cartilage. Binding of anti-Gal to the α-gal nanoparticles will activate the complement system and induce a rapid and extensive recruitment of macrophages into these sites of damage and further activation of these macrophages by the interaction between the Fc portion of the anti-Gal antibody coating the α-gal nanoparticles and Fcγ receptors on the macrophages. The activated macrophages will secrete cytokines/growth factors that recruit stem cells which will differentiate into chondroblasts upon interaction with the fragmented cartilage ECM applied with the α-gal nanoparticles within the applied paste. These chondroblasts, in turn, secrete collagen and other cartilage matrix proteins and glycosaminoglycans, resulting in repair and remodeling of the damaged cartilage. The fragmented cartilage applied in the paste with α-gal nanoparticles may be of autologous origin, allogeneic origin or of xenogeneic origin (e.g. bovine or porcine origin). If the fragmented cartilage is of xenogeneic origin, it should lack α-gal epitopes. This can be achieved either by using cartilage from an α1,3galactosyltransferase knockout (KO) animal donor, or by enzymatic destruction of the α-gal epitopes on the xenogeneic cartilage by incubation of the cartilage in a solution of recombinant α-galactosidase (Stone et al. Transplantation 83:211, 2007). The recruited chondroblasts developing from the recruited stem cells will, in turn, secrete collagen and other cartilage matrix proteins and glycosaminoglycans, resulting in repair and remodeling of the damaged cartilage. An illustration of such recruitment of stem cells which differentiate into cartilage producing fibrochondroblasts is presented in FIG. 33. In the same embodiment the α-gal nanoparticles may be introduced into allogeneic or xenogeneic meniscus cartilage by injection or any other method and implanted in patients that undergo meniscectomy. The macrophages recruited into the implanted meniscus will be activated and will secrete cytokines/growth factors that recruit stem cells into the implanted meniscus. The recruited stem cells will be instructed by the meniscus ECM to differentiate into chondrofibrocytes that secrete collagen and other ECM molecules ultimately resulting in regeneration of the implanted meniscus into an autologous meniscus that functions in the recipient for many years.

In yet another embodiment, application of α-gal nanoparticles to bone injuries such as bone fractures or interface with bone implants induces rapid and extensive recruitment of macrophages into these sites of damage. In injured bones, administered α-gal nanoparticles recruit and activate macrophages which secrete cytokines/growth factors that recruit stem cells becoming osteoclasts and osteoblasts upon interaction with the bone ECM. These osteoblasts and osteoclasts mediate repair and remodeling of the damaged bone. It is further contemplated that a mixture of α-gal nanoparticles and a homogenate consisting of fragmented bone in the form of paste made of any filler known to those skilled in the art or without a filler is to be applied to bone injuries such as bone fractures or interface with bone implants. The rapid and extensive recruitment of macrophages into these sites of damage and activation of these macrophages by the interaction between the Fc portion of the anti-Gal antibody coating the α-gal nanoparticles and Fcγ receptors on the macrophages result in the recruitment of stem cells. The stem cells receive cues from the fragmented bone in the paste and the fractured bone ECM to differentiate into osteoblasts and osteoclasts that may mediate repair and remodeling of the damaged bone. The bone fragments in the paste may be of autologous, allogeneic or xenogeneic source. Bone fragments of xenogeneic source should be devoid of α-gal epitopes either by obtaining the bone from KO pigs or treated with recombinant α-galactosidase.

In another embodiment, α-gal nanoparticles may be used for the therapy of damaged lungs in patients with a variety of respiratory diseases, including, but not limited to lung damage because of smoking cigarettes and asbestosis. α-Gal nanoparticles may be administered into damaged lungs by inhalation of an aerosolized suspension of these nanoparticles. Such inhalation will result in the deposition of α-gal nanoparticles in the surfactant coating the alveoli and in the mucus secretion coating the bronchioles, bronchi and trachea (the airways). The binding of anti-Gal to these nanoparticles will activate the complement system and induce chemotactic recruitment of macrophages onto the surface of damaged alveoli (air sacs) and airways and activation of these macrophages. The macrophages activated by the α-gal nanoparticles will secrete cytokines/growth factors that induce recruitment of stem cells and which may enable prolonged survival of the recruited stem cells. It is contemplated that these stem cells will be induced by the microenvironment and the ECM within the treated alveoli to differentiate into pneumocytes and other cells of the alveoli and thus regenerate the damaged alveoli and/or form new alveoli. Within the airways, α-gal nanoparticles reaching the mucus film coating bronchioles, bronchi and trachea, will recruit and activate the macrophages as in the alveoli. These activated macrophages may recruit stem cells that differentiate into the ciliated epithelium and mucus secreting cells that comprise the normal epithelium of the airways.

XI. Supporting the Viability and Function of Stem Cells and of Mature Cells Converted into Stem Cells by their Co-Administration with α-Gal Nanoparticles

A large proportion of the research in tissue repair and regeneration focuses currently on the administration of stem cells of various origins and of mature cells converted into stem cells into injury sites or into damaged tissues in order to achieve repair and regeneration of the target tissue. Conversion of mature cells into stem cells has been achieved by various methods including but not limited to stressing the cells by low pH shock (Okobata et al. Nature 505: 641, 2014). When administered into injured sites, the stem cells and mature cells converted into stem cells display survival for limited periods of time that may not be long enough to enable effective conversion into the cells that regenerate the injured tissue (Lesaulet et al. PLoS One 7: e46698, 2012). In another embodiment this invention teaches the formation of a microenvironment that is conducive for prolonged survival of the stem cells or of mature cells converted into stem cells by administration of these cells together with α-gal nanoparticles. When such stem cells are administered within a suspension also containing α-gal nanoparticles, the interaction of the administered α-gal nanoparticles with the anti-Gal antibody activates the complement system proteins diffusing into the site of administered stem cells or cells converted into stem cells. The activated complement system produces complement cleavage peptides that are chemotactic factors. These chemotactic factors recruit macrophages which are activated following binding the Fc portion of anti-Gal on the α-gal nanoparticles. The activated macrophages secrete a wide range of cytokines/growth factors which facilitate the survival of the stem cells and of cells converted into stem cells for periods long enough to enable their effective differentiation into cells that regenerate the injured tissue.

XII. Incorporation of α-Gal Nanoparticles into Decellularized Tissues for Organ Regeneration

In the recent two decades there has been extensive research in the generation of biodegradable natural biomaterials which may be used in tissue engineering for tissue repair and regeneration (Atala, Current Opinion Biothecnol 20:575, 2009; Badylack Lancet 379: 943, 2012). One exciting direction is the possible use of decellularized tissues and organs containing extracellular matrix (ECM) that maintains the original scaffold architecture and composition. Conservation of the ECM in whole organs is being achieved by advanced dynamic decellularization techniques using combination of various detergents and DNA destroying solutions (Atala, supra; Badylak, supra). The ECM in decellularized tissues and organs instructs stem cells to differentiate into cells that restore the biological function of the injured tissue. A number of studies have shown the use of scaffold derived from decellularized porcine bladder submucosa for urethral tissue engineering (Liu et al. Biomaterials 30:3865, 2009), porcine decellularized myocardium for regeneration of the injured ventricular wall (Sarig et al. Tissue Eng Part A 18:2125, 2012) and porcine small intestinal submucosa for repair of small bowel tissue (Chen and Badylak, J Surg Res 99:352, 2001). Because of the decellularization processing, these biomaterials are porous (Crapo et al. Biomaterials 32:3233, 2011). In another embodiment, soaking such biomaterials in an α-gal nanoparticles suspension will result in the nanoparticles being absorbed into these biomaterials and accelerate the regeneration of the tissues formed by them following implantation. Use of freeze dried biomaterials may further increase α-gal nanoparticles penetration into these biomaterials. In addition, when whole decellularized organs are used as biomaterials for tissue engineering, α-gal nanoparticles penetration can be achieved throughout the organ by perfusion with the α-gal nanoparticles suspension, similar to the perfusion of the decellularizing detergent solutions (Crapo et al. Supra). Following implantation, these nanoparticles will bind anti-Gal since the antibody is ubiquitous in the body and diffuses into the implant. The resulting complement activation will induce rapid migration of the macrophages into the implant as demonstrated in FIG. 34 and FIG. 35. Activation of these recruited macrophages by anti-Gal coated α-gal nanoparticles will induce secretion of cytokines/growth factors by these macrophages and the resulting recruitment of stem cells by these secreted cytokines/growth factors. The recruited stem cells will be guided by the ECM to differentiate into the original cells of the decellularized tissue, similar to the differentiation of stem cells into meniscus cartilage within PVA sponge discs illustrated in FIG. 33. Thus, the α-gal nanoparticles within decellularized or non-decellularized implants can shift the dynamics of cell repopulation from fibroblasts infiltration and fibrosis as a default regenerative process, to the recruitment of stem cells that differentiate under the guidance of the ECM into the desired cells that restore the normal biological activity of the organ. As non-limiting examples, the presence of α-gal nanoparticles within a heart engineered tissue patch may result in repopulation of the patch with macrophages, then stem cells differentiating into cardiomyocytes that assist in the contraction of the ventricular myocardium, whereas the presence of α-gal nanoparticles in decellularized urinary bladder tissue may result in the repopulation of the implant with smooth muscle cells and the covering of the surface with transitional epithelium thereby restoring the normal architecture of the urinary bladder tissue.

If the natural biomaterial is of a nonprimate mammalian origin, such as of porcine origin, the engineered tissue to be used for such implant should be enzymatically stripped of autologous α-gal epitopes by α-galactosidase, or should originate from α1,3galactosltransferase knockout pigs (KO pigs) devoid of α-gal epitopes. Since the α-gal epitope is present both on cells and on glycoproteins of the ECM (e.g. laminin), binding of anti-Gal to these epitopes on the ECM may result in immune mediated destruction of the ECM. Therefore, stripping the α-gal epitope from the ECM, or using tissues and organs that lack it will help in preserving the ECM upon implantation in humans.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: kDa (kilodalton); rec. (recombinant); N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); l or L (liters); ml (milliliters); μl (microliters); cm (centimeters); mm (millimeters); μm (micrometers); nm (nanometers); C (degrees Centigrade); α1,3GT (α1,3galactosyltransferase); BSA (bovine serum albumin); ELISA (enzyme linked immunosorbent assay); FcγR-Fcγ receptors; HRP (horseradish peroxidase) IFNγ (interferon-γ); knockout (KO); mAb (monoclonal antibody); OD (optical density); OPD (ortho phenylene diamine); PBS (phosphate buffered saline); RBC (red blood cells).

### Example 1

### Production of α-Gal Liposomes and Binding of Anti-Gal by these Liposomes

Exemplary α-gal liposomes are generated from extracts of rabbit red blood cell (RBC) membranes. These membranes are used since they contain glycolipids carrying from one to more than seven α-gal epitopes per molecule as disclosed in Eto et al., Biochem. (Tokyo) 64, 205, (1968); Stellner et al., Arch. Biochem. Biophys. 133, 464 (1973); Dabrowski et al., J. Biol. Chem. 259, 7648 (1984) and Hanfland et al., Carbohydr. Res. 178, 1 (1988), all of which are hereby incorporated by reference. However, α-gal liposomes may be produced from any natural or synthetic source of α-gal glycolipids upon addition of phospholipids in the presence or absence of cholesterol, after processing as described herein. As a non-limiting example, rabbit RBC are used at a volume of 0.25 liter packed cells. The RBC are lysed by repeated washes with distilled water. The rabbit RBC membranes are then mixed with a solution of 600 ml chloroform and 900 ml methanol for 20 h with constant stirring to dissolve the membrane glycolipids, phospholipids and cholesterol into the extracting solution. In contrast, proteins are denatured and are precipitating within and upon the membranes. Subsequently, the mixture is filtered to remove non-solubilized fragments and denatured proteins precipitates. The extract contains the rabbit RBC phospholipids, cholesterol and glycolipids, dissolved in the organic solution of chloroform and methanol (FIG. 2A). With the exception of the glycolipid ceramide tri-hexoside (CTH) having the structure Galα1-4Galβ1-4Glc-Cer, the glycolipids extracted from rabbit RBC membranes generally have 5 to more than 25 carbohydrate units in their carbohydrate chains with one or several branches, all of which are capped with α-gal epitopes. Rabbit RBC glycolipids were also reported to have 30, 35 and even 40 carbohydrate units with α-gal epitopes on their branched carbohydrate chains as provided for in Honma et al., J. Biochem. (Tokyo) 90, 1187 (1981), incorporated in its entirety by reference. The extract containing glycolipids, phospholipids and cholesterol is subsequently dried in a rotary evaporator. The amount of dried extract is approximately 300 mg per 0.25 liter of packed rabbit RBC.

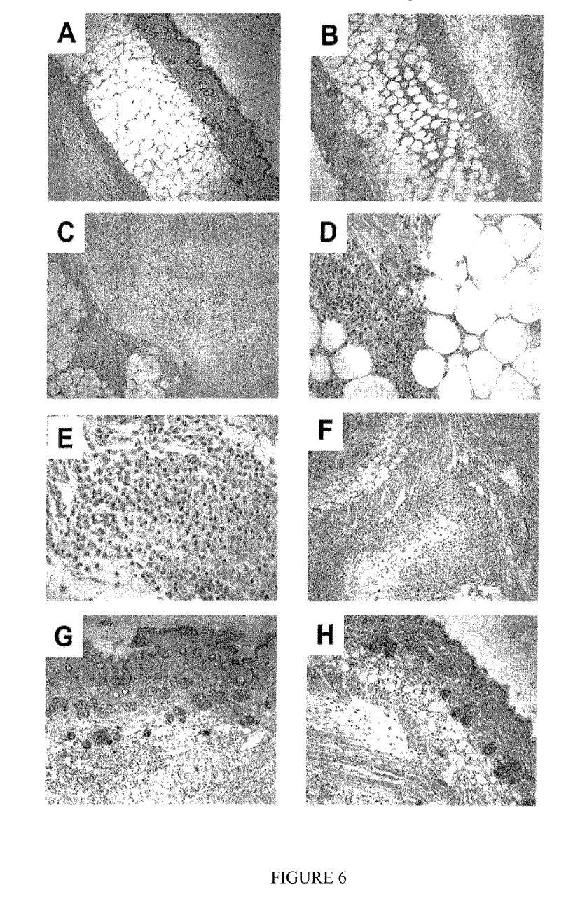

Thirty ml of saline is added to the dried extract, which is then subjected to sonication in a sonication bath. This sonication process results in conversion of the extract into liposomes comprised of α-gal glycolipids, phospholipids and cholesterol, referred to as α-gal liposomes, as schematically illustrated in FIG. 1A. Generally, α-gal liposomes may be of any size including, but not limited to, the range of 50 nanometer (nm) to 100 micrometer (μm). Preferably, α-gal liposomes may have a size in the range of 0.1-20 μm, with an average size controlled by the length and intensity of the sonication process. Because the α-gal epitopes of many of the α-gal glycolipids protrude out of the liposomes, these epitopes readily interact with anti-Gal antibodies. This interaction results in activation of the complement cascade by anti-Gal binding to α-gal liposomes and the generation of C5a, C4a and C3a complement fragments, which in turn, form a chemotactic gradient that directs the migration of neutrophils, monocytes and macrophages from the circulation and from the peri-vascular space into the site of the α-gal liposome depot. The inflammatory cell infiltrate is readily observed in the histological sections of FIG. 6. The neutrophils and macrophages are capable of destroying microbial agents such as bacteria, viruses or fungi in the region of the injected α-gal liposomes. Macrophages have Fcγ receptors (FcγR) that bind to the Fc portion of IgG molecules that have bound to antigen. In this way, anti-Gal IgG molecules that bind to α-gal epitopes on the α-gal liposomes, also bind to FcγR on the recruited macrophages, as schematically illustrated in FIG. 1B. This interaction results in activation of the macrophage, internalization of the α-gal liposomes and secretion of a wide variety of growth factors, cytokines and chemokines which orchestrate the healing and remodeling of damaged tissue in part by recruiting fibroblasts and mesenchimal stem cells and stimulate proliferation of epithelial cells.

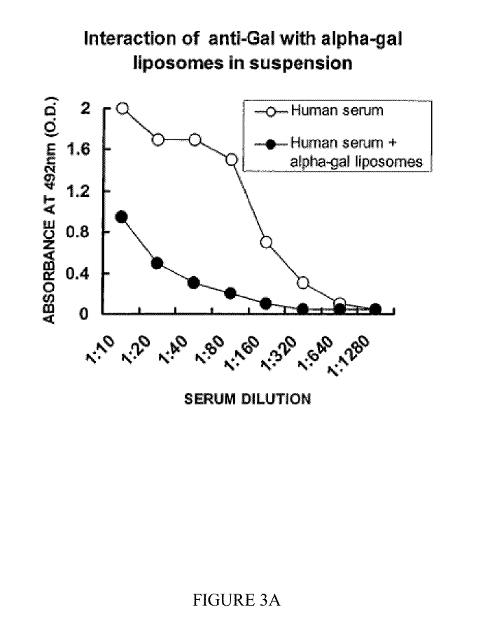

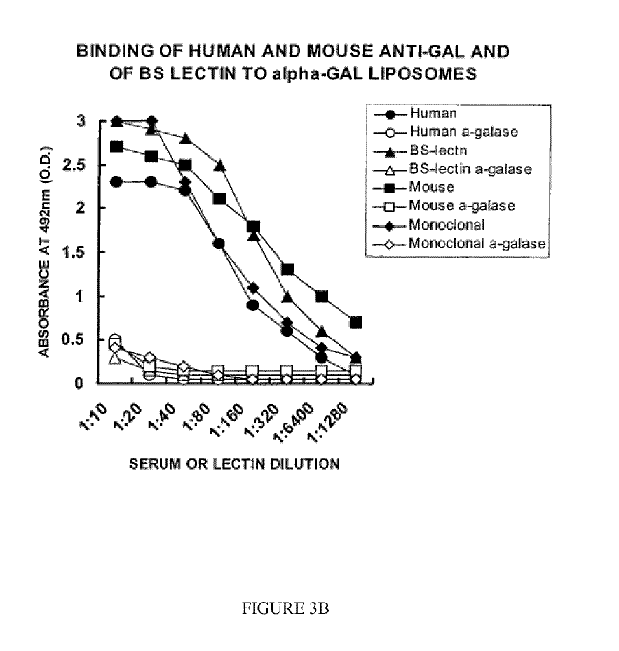

The specific binding of anti-Gal of human and mouse origin to the exemplary α-gal liposomes is graphically depicted in FIG. 3. Specifically, FIG. 3A shows the binding of anti-Gal to α-gal liposomes in suspension. When tested for binding to synthetic α-gal epitopes linked to bovine serum albumin (α-gal BSA) as solid-phase antigen, binding at a level higher than 1.0 optical density (OD) could be observed at serum dilutions of up to 1:80. However, if the serum was pre-incubated for 2 h at 37° C. with 10 mg/ml of α-gal liposomes, subsequent binding to the solid-phase α-gal BSA was less than 1.0 OD even at the lowest serum dilution of 1:10. This indicates that much of the serum anti-Gal binds to α-gal liposomes in suspension and therefore it is neutralized and is unavailable for the subsequent binding to the α-gal BSA as solid-phase antigen in the ELISA.

Similarly, FIG. 3B shows the binding of human and mouse anti-Gal to α-gal liposomes that serve as a solid-phase antigen in an ELISA. The α-gal liposomes were plated as 50 aliquots of a 100 μg/ml suspension in phosphate buffered saline (PBS) in ELISA wells and dried overnight. The drying results in the firm adhesion of the α-gal liposomes to the wells. The wells were subsequently blocked with 1% BSA in PBS. Human serum, α1,3galactosyltransferase (α1,3GT) knockout (KO) mouse serum containing anti-Gal antibody, and mouse monoclonal anti-Gal as disclosed in Galili et al., Transplantation 65, 1129 (1998), hereby incorporated by reference, were added to the wells. The KO mouse serum contains anti-Gal antibodies upon immunization of the mouse with pig kidney membranes as provided for in Tanemura et al., J. Clin. Invest. 105, 301 (2000), hereby incorporated by reference. After 2 h incubation, the wells containing human serum at various dilutions were washed and binding of anti-Gal to α-gal liposomes was determined by the addition of the corresponding horseradish peroxidase (HRP) coupled anti-human, or anti-mouse secondary antibody followed by color reaction with ortho phenylene diamine (OPD). Anti-Gal readily binds to α-gal liposomes, with 1.0 OD value at a serum dilution of 1:160 (). The specificity of the anti-Gal/α-gal liposome interaction was demonstrated be eliminating anti-Gal binding upon treatment of the α-gal liposomes coating the ELISA wells with recombinant α-galactosidase (◯) as disclosed in Stone et al., Transplantation 83, 201 (2007), incorporated herein by reference. The α-galactosidase enzyme cleaves the terminal galactose unit from the α-gal epitope, thereby destroying this epitope. Following such enzymatic treatment, the binding of anti-Gal to the liposomes could not be detected. The anti-Gal specific binding to α-gal liposomes was also demonstrated using serum from immunized KO mice (▪), whereas treatment of the α-gal liposomes with α-galactosidase eliminated anti-Gal binding (□). Specific binding of α-gal epitopes by α-gal liposomes was also observed using an anti-Gal mAb from hybridoma cell supernatants (♦). As expected, the anti-Gal mAb did not bind to α-gal liposomes after treatment with α-galactosidase (⋄). Similar specific binding to α-gal liposomes was observed with the α-gal epitope reactive lectin Bandeiraea simplicifolia IB4 (BS lectin) (▴) as provided for in Wood et al., Arch. Biochem. Biophys. 198, 1 (1979), incorporated herein by reference. The binding of this lectin was also abolished by treatment with α-galactosidase (Δ). These observations clearly demonstrate that the α-gal liposomes produced by sonication of chloroform/methanol extracts from rabbit RBC membranes, readily bind to anti-Gal antibodies. Although it is not necessary to understand the mechanism of an invention, it is believed that binding of anti-Gal to α-gal liposomes occurs in vivo at the injection site in subjects possessing anti-Gal antibodies. It should be noted that binding studies with α-gal liposomes that were reduced in size into submicroscopic α-gal nanoparticles provided identical results as those reported in FIG. 3.

### Example 2

### Binding of Anti-Gal to α-Gal Liposomes Induces Complement Activation

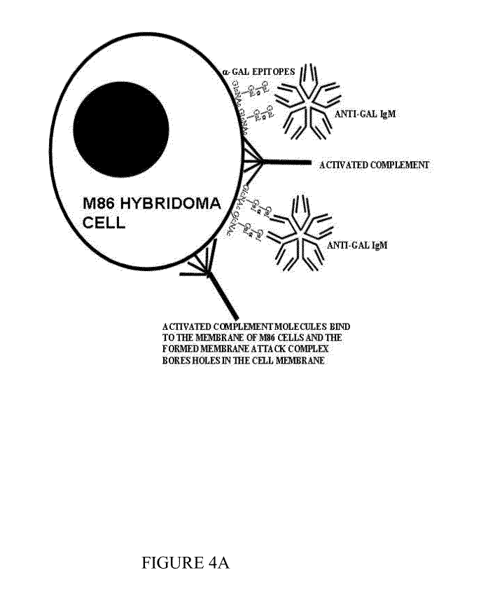

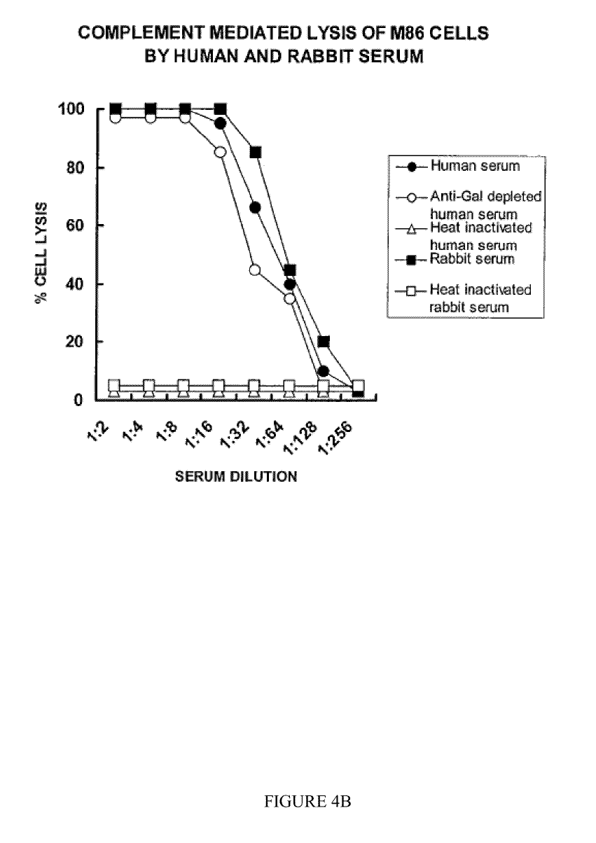

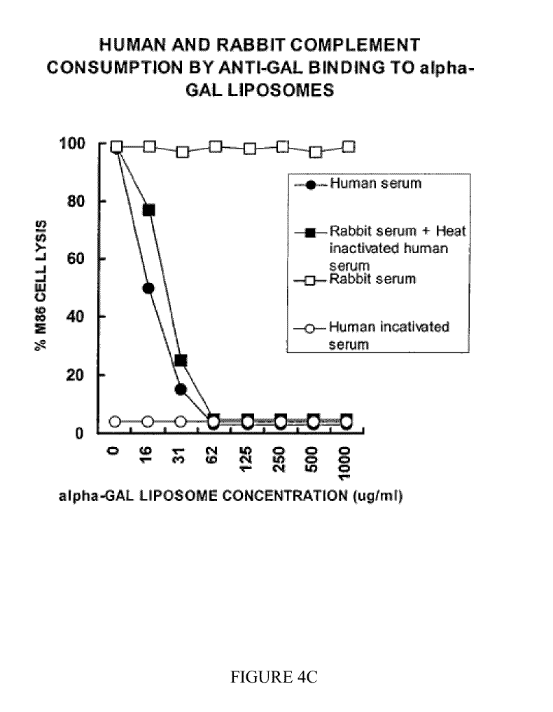

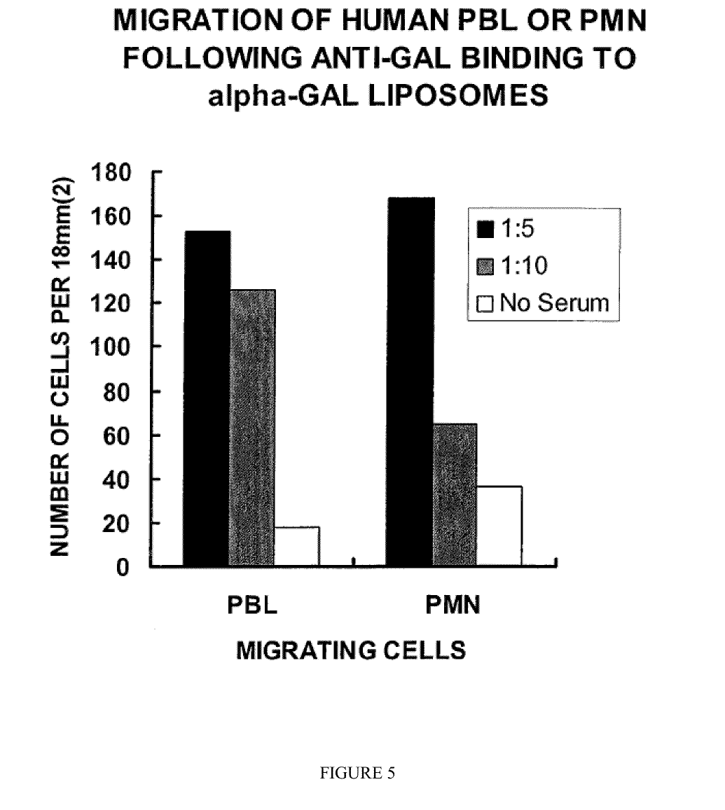

This example describes the activation of complement within serum as a result of the binding of serum anti-Gal antibodies to α-gal epitopes on α-gal liposomes. Complement activation was observed herein by measuring the consumption of complement (e.g., loss of complement ability to lyse cells with bound antibodies). The binding of anti-Gal to α-gal epitopes on α-gal liposomes results in complement consumption due to conversion of the activated complement into complement fragments. The hybridoma cell line M86, which secretes an anti-Gal mAb, was used as a readout system for measuring complement mediated cytolysis (e.g., presence of complement in the serum). Since M86 cells express α-gal epitopes, the anti-Gal IgM mAb they produce bind to the α-gal epitopes on the hybridoma cell surface as schematically illustrated in FIG. 4A. When complement is added, it is activated by the anti-Gal bound to α-gal epitopes on the M86 cells, ultimately resulting in complement mediated lysis of the M86 cells as provided for in Galili et al., Transplantation 65, 1129 (1998), hereby incorporated by reference. Incubation of M86 cells with human serum at various dilutions, for 1 h at 37° C. () results in 100% lysis at serum dilutions of at least 1:8 and more than 40% lysis of the M86 cells even at a serum dilution of 1:64 (FIG. 4B). Lysis of M86 cells does not require exogenous anti-Gal since these cells have autologous anti-Gal bound to the α-gal epitopes of the cell surface. Thus, human serum depleted of anti-Gal also induces M86 lysis, due to the complement activity present in human serum (◯). Anti-Gal depletion can be achieved by incubation of the human serum with glutaraldehyde fixed rabbit RBC, which express an abundance of α-gal epitopes. The adsorption of anti-Gal on fixed rabbit RBC was performed on ice to prevent complement activation during the adsorption process. Rabbit serum (which lacks anti-Gal antibody, similar to serum from all other nonprimate mammals) has complement and thus can lyse 100% of M86 cells at at least 1:8 dilution and lyse more than 40% M86 cells even at a dilution of 1:64. Incubation at 56° C. for 30 min of both human serum (Δ) and rabbit serum (□) results in inactivation of complement and hence loss of lytic activity (FIG. 4B).

Addition of α-gal liposomes to the human serum diluted 1:10 for 30 min at 37° C., prior to addition of M86 cells, resulted in the loss of complement mediated cytolysis of the M86 cells even at a concentration of 62 μg/ml of the α-gal liposomes (FIG. 4C). Loss of lytic activity is presumed to occur as a result of the consumption of serum complement due to anti-Gal binding to α-gal liposomes. Thus, subsequent addition of M86 cells and incubation of the mixture for 1 h at 37° C. results in no significant M86 cell cytolysis, whereas in the absence of α-gal liposomes the complement in human serum lyses 100% of the M86 cells. Similarly, the complement in normal rabbit serum diluted 1:10 lyses more than 95% of M86 cells. However if the rabbit serum is incubated with α-gal liposomes and with heat inactivated human serum, no significant M86 lysis is observed when these cells are added to suspensions containing 62 μg/ml of α-gal liposomes. This lack of cell lysis is the result of the rabbit complement consumption due to the human anti-Gal binding to the α-gal liposomes and consumption of the rabbit complement, prior to the addition of M86 cells. These data indicate that binding of anti-Gal to α-gal liposomes in vivo will also result in complement activation and therefore to the generation of C5a, C4a and C3a chemotactic factors, which are always part of the complement activation process.

### Example 3

### Induction of Monocyte and Macrophage Migration

This example describes the chemotactic gradient generated by complement activation as a result of serum anti-Gal binding to α-gal liposomes. The generation of complement cleavage chemotactic factors was assessed by monitoring the migration of monocytes and macrophages in a Boyden chamber. This system includes two chambers, a lower chamber containing serum mixed with α-gal liposomes and an upper chamber containing various white blood cells. The two chambers are separated by a porous filter that permits the migration of cells from the upper to the lower chamber via pores within the filter. At the end of a 24 h incubation period at 37° C. the filters are stained and the number of migrating cells (e.g., within lower chamber) is counted. The study shown in FIG. 5 was performed with 10⁶ cells/ml in the upper chamber and serum diluted 1:5 (black columns) or 1:10 (gray columns) and mixed with 1 mg/ml of α-gal liposomes in the lower chamber. A negative control solution in the lower chamber contained medium and α-gal liposomes, in the absence of serum, in order to assess the random migration of cells (open columns). Incubation of human peripheral blood lymphocytes (PBL, referring to blood mononuclear cells, including monocytes) or polymorphonuclear cells (PMN) in the upper chamber and α-gal liposomes in the absence of serum in the lower chamber did not induce significant cell migration. However when serum and α-gal liposomes were mixed together in the lower chamber, extensive migration of mononuclear cells and neutrophils was observed toward the lower chamber. The morphology of the migrating cells in the PBL population indicated that the majority of the migrating cells were monocytes.

### Example 4

### Intradermal Recruitment of Neutrophils, Monocytes and Macrophages

In vivo studies on the effect of α-gal liposomes were performed in α-1,3galactosyltransferase knockout (KO) mice as provided for in Thall et al., J. Biol. Chem. 270, 21437-21442 (1995), incorporated in its entirety by reference, since these are the only non-primate mammals capable of producing anti-Gal antibodies. All other non-primate mammals (with the exception of KO pigs) express α-gal epitopes and thus do not produce anti-Gal antibodies. In order to monitor the in vivo effect of anti-Gal interaction with injected α-gal liposomes, KO mice producing anti-Gal (e.g., KO mice pre-immunized with 50 mg pig kidney membranes resulting in the induction of anti-Gal titers similar to those observed in humans) were injected intradermally with 1.0 mg α-gal liposomes in 0.1 ml saline.

Skin specimens from the injection site were obtained at different time points, fixed, stained with hematoxyllin-eosin (H&E) and inspected under a light microscope. FIG. 6A depicts normal skin prior to injection of α-gal liposomes. The epidermis comprises of one to two layers of epithelial cells. The dermis contains fibroblasts under the epidermal layer, fat cells as a deeper layer and an underlying narrow layer of muscle cells and fibroblasts. No inflammatory cells are observed in the normal skin (×100). FIG. 6B depicts the skin 12 h after injection of 1.0 mg α-gal liposomes. The intradermal injection site is identified as the area with the least amount of cells, under the muscle cell layer. Note that at this early time point the injection area is filled with neutrophils that surround the injected site both within the fat cell layer and within the side adjacent to the epidermis (×100). FIG. 6C also depicts the skin 12 h post-injection. The α-gal liposome depot of the injection site is shown in the center of image, which is bordered on the left by the fat cell layer. The α-gal liposome injection site has a low density of dermal cells. However, by 12 h post-injection, the injection site has become populated by infiltrating inflammatory cells presumably recruited by the injected α-gal liposomes bind to anti-Gal antibody and complement activation. A higher magnification of the infiltrating cells within the fat cell area in FIG. 6D (×400) indicates that the infiltrating cells are neutrophils. The extensive migration of neutrophils into the α-gal liposome injection site is followed by migration of monocytes and macrophages, which are recruited by the locally produced complement chemotactic factors. FIG. 6E depicts the α-gal liposome injection site 48 h post-injection. As shown in this higher magnification (×400) most of the infiltrating inflammatory cells in the injection site are mononuclear cells with nuclear features resembling macrophages (e.g., kidney shaped nuclei). These cells are evident in the injection site already 24 h post-injection. The characterization of these cells as macrophages is further described in Example 6 below. FIG. 6F depicts the α-gal liposome injection site 5 days post-injection. By this time the injection site is filled with large round macrophages, reflecting the local activation of the infiltrating macrophages due to the interaction of the anti-Gal opsonized α-gal liposomes. Only the center of the injection site is devoid of macrophages, and likely functions as an α-gal liposome depot. The epidermis in this figure is shown in the upper left corner. As shown in FIG. 6G, infiltrating macrophages are detectable in the injection site as late as 14 days post-injection. As shown in FIG. 6H, macrophages completely disappear from the injection site by day 20 post-injection. The injection site at that stage is rich with fibroblasts and muscle cells, which are contemplated to have originated from myofibroblasts recruited by the macrophages activated by the anti-Gal opsonized α-gal liposomes. Nonetheless an understanding of the mechanism is not necessary in order to make and use the present invention. The histological analysis presented in FIG. 6 indicates that intradermal injection of α-gal liposomes is suitable for induction of recruitment of neutrophils and macrophages. The recruitment is detectable within 12 h by the extensive neutrophils infiltration, followed by a second wave of infiltrating monocytes and macrophages within 24-48 h post-injection. In skin wounds accompanied by microbial infection, the neutrophils and the macrophages recruited by the interaction between anti-Gal and the injected α-gal liposomes are contemplated to mediate the destruction of the infectious agent. In addition, the various growth factors, cytokines and chemokines secreted by the activated macrophages are contemplated to mediate wound healing and repair of the damaged tissue. Nonetheless an understanding of the mechanism(s) is not necessary in order to make and use the present invention.

### Example 5

### α-Gal Liposomes do not Elicit an Immune Response

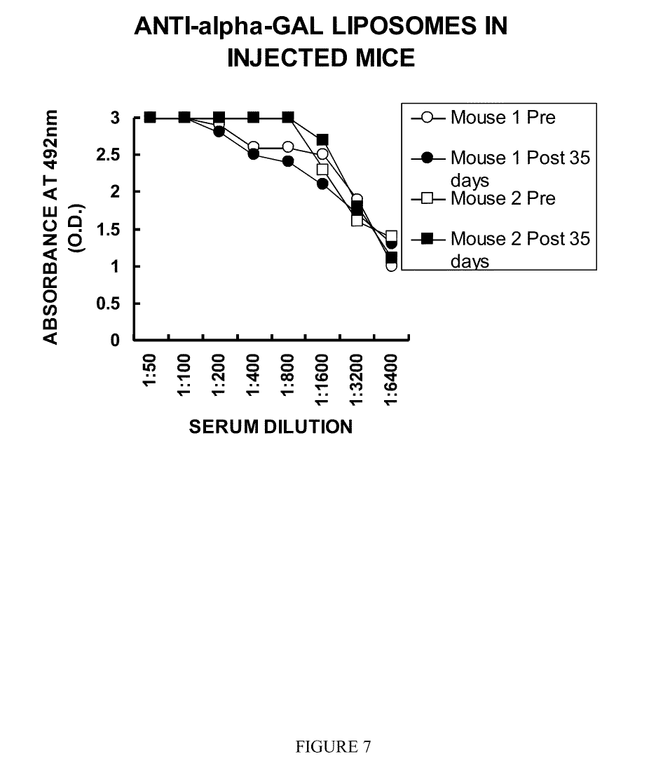

Although α-gal liposomes readily bind in vitro and in vivo to anti-Gal antibodies, they do not elicit an immune response against the injected α-gal liposomes as determined by ELISA. To demonstrate this, 50 μl of a solution containing α-gal liposomes at a concentration of 100 μg/ml were dried in ELISA wells to serve as a solid phase antigen. Serum samples from two representative mice obtained before (◯ and □) and 35 days post intradermal injection ( and ▪, respectively) were tested for IgG binding to α-gal liposomes. A humoral immune response against components of the α-gal liposomes should result in increased IgG binding to α-gal liposome-coated wells in post-injection serum (e.g., higher activity as compared to pre-injection serum). As shown in FIG. 7, the binding of IgG antibodies to α-gal liposomes 35 days post-injection was similar or lower to that observed prior to injection. Thus administration of α-gal liposomes does not elicit a deleterious humoral immune response against the injected material, despite their ability to recruit neutrophils, monocytes and macrophages to the injection site.

### Example 6

### Recruitment of Macrophages into Polyvinyl Alcohol (PVA) Sponges by α-Gal Liposomes

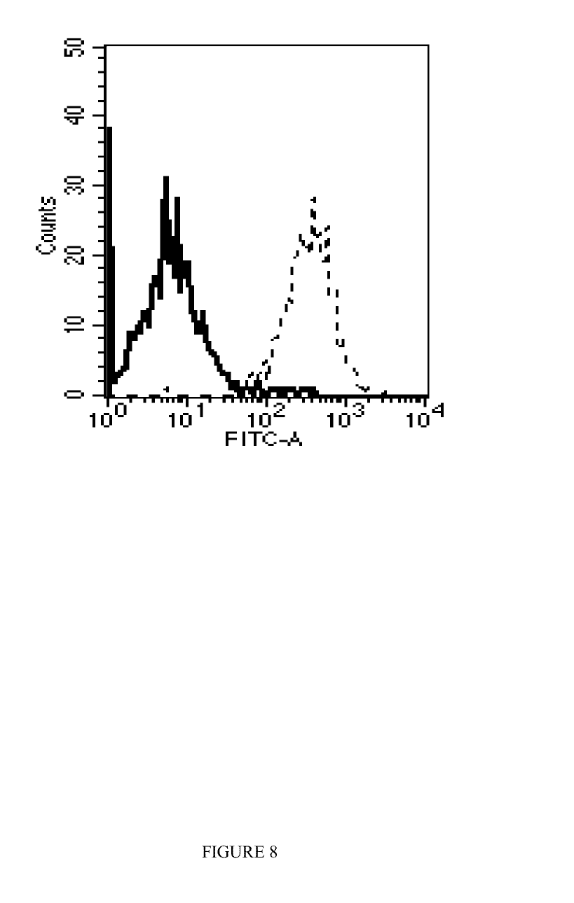

The objective in this study was to determine whether the mononuclear cells recruited by injected α-gal liposomes binding the anti-Gal antibody (FIG. 6) are macrophages that can be identified by immunostaining and analysis of stained cells by flow cytometry. This was performed by the use of subcutaneously implanted polyvinyl alcohol (PVA) sponge discs (PVA Unlimited, Inc., 10 mm diameter and 3 mm thickness). Prior to implantation, the discs were soaked in a suspension of α-gal liposomes (100 mg/ml). α1,3galactosyltransferase knockout mice (KO mice) were anaesthetized with 0.04 cc of ketamine/xylazine (50 mg/cc and 2.5 mg/cc, respectively). The dorsa of the mice are shaved and a 10 mm linear incision was made then implanted subcutaneously with the PVA disc soaked in α-gal liposome suspension. The wound was closed by suture. The PVA discs were removed from the mice 72 h post implantation. The present invention teaches that anti-Gal binds to the α-gal liposomes, activates complement and recruit inflammatory cells into the PVA sponge discs. The cells migrating in vivo into the PVA sponge discs were retrieved by repeated pressing on sponge discs immersed in PBS. Subsequently, the cells were washed, stained with the mouse monoclonal anti-CD11b macrophage specific antibody (Pharmingen Inc, CA) and subjected to flow cytometry (FACS). As shown in FIG. 8, all infiltrating cells were found to be macrophages, since all cells displayed shift to the right after staining with anti-CD11b antibody (broken line) in comparison to isotype control (solid line). Thus, >99% infiltrating cells were stained positively with the macrophage specific monoclonal antibody implying that the cells infiltrating the disc containing the α-gal liposomes were macrophages. PVA discs soaked with saline and studied 3 days post implantation contained no measurable numbers of infiltrating cells.

### Example 7

### Effects of α-Gal Ointment Application on Wound Healing

The α-gal ointment is another composition containing α-glycolipids that can be used for accelerated wound healing by recruitment of macrophages to the damaged area. It is of particular beneficial use in skin burns. The α-gal ointment is prepared by mixing α-gal glycolipids with Vaseline or any other cream or gel at a final concentration ranging from 0.001% to more than 90% α-gal glycolipids. The α-gal glycolipids may or may not be purified from the mixture with phospholipids and cholesterol obtained by extraction from rabbit RBC membranes (described in Example 1). The α-gal ointment is applied topically onto such as, but not limited to skin burns. Burns may be caused by various injuries (e.g., hot objects, hot fluids or radiation). The illustration in FIG. 9 describes treatment of a burn with α-gal ointment. This treatment is applicable to other types of wounds as well. The natural anti-Gal antibody and complement proteins are among the serum proteins that leak from the damaged blood vessels into the burn area because of their high concentration in the serum. As illustrated in FIG. 9, the interaction of the natural anti-Gal antibody in the burn with the large amounts of α-gal glycolipids in the ointment induces local activation of the complement cascade, and thus, generates the complement cleavage chemotactic fragments such as C5a and C3a that recruits macrophages to the area of this antibody binding to its antigen. This extensive recruitment of macrophages, which is much faster than the physiologic migration of macrophages into burns, results in accelerated debridement, epithelialization, fibroblasts migration and proliferation and collagen matrix deposition by the fibroblasts, ultimately resulting in accelerated healing of burns and shorter morbidity than that achieved with current treatments. This treatment is applicable to various skin injuries where anti-Gal will leak from damaged capillaries and thus will interact with α-gal glycolipids within the applied α-gal ointment. α-Gal ointment may also be formed with ointments containing antibiotics (as those presently used for burns treatment), thus preventing infections while the healing process occurs. This treatment of topical application of α-gal glycolipids in an ointment formulation introduces no chemicals, other than the natural α-gal epitopes on glycolipids. Phospholipids and cholesterol, if present, are identical to those in human cells. Therefore, this treatment is likely to be safe. The safety of α-gal glycolipids is further implied from the fact that humans are constantly exposed to α-gal epitopes via a wide range of foods containing beef and pork meat, without any adverse effects.

### Example 8

### Binding of Anti-Gal to α-Gal Glycolipids in α-Gal Ointment

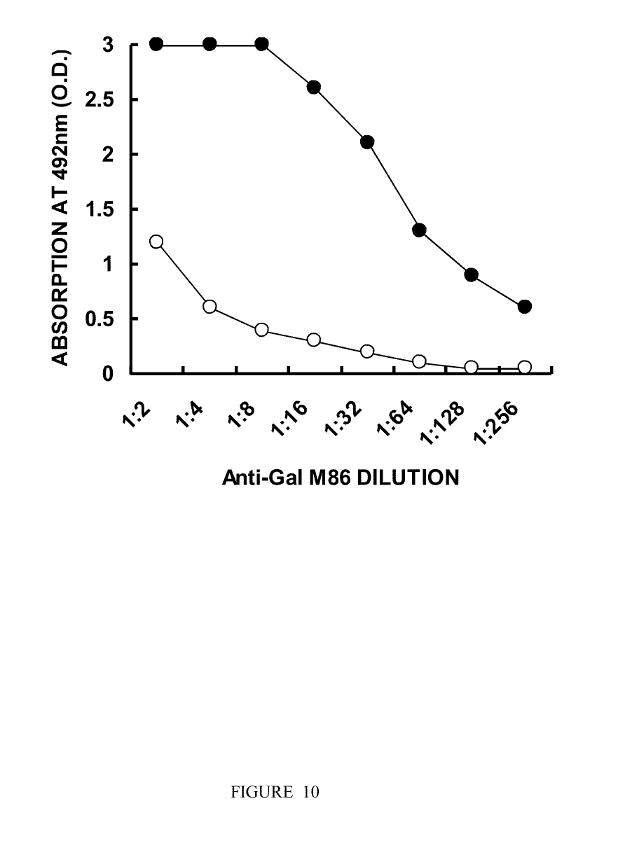

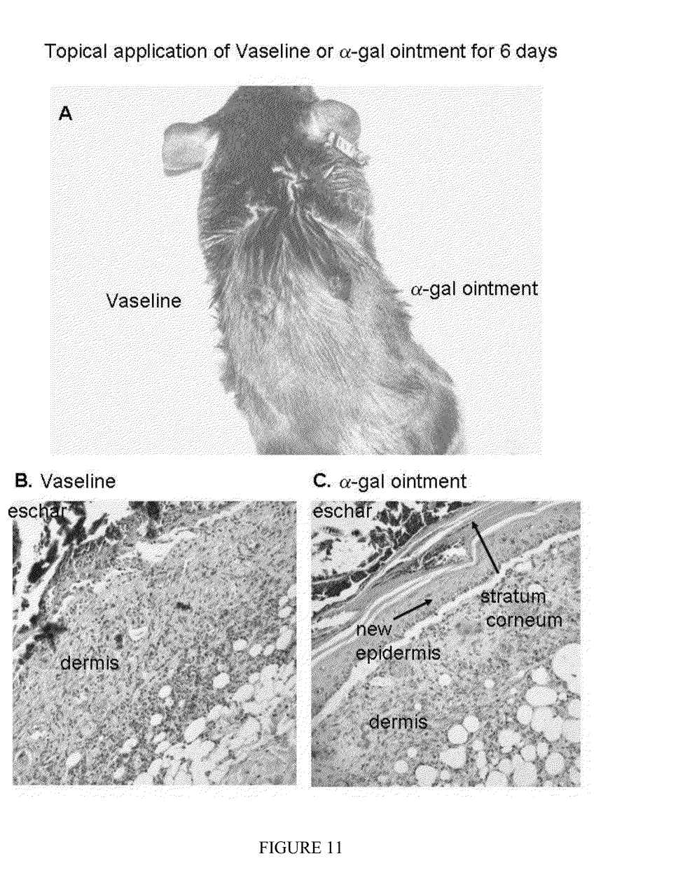

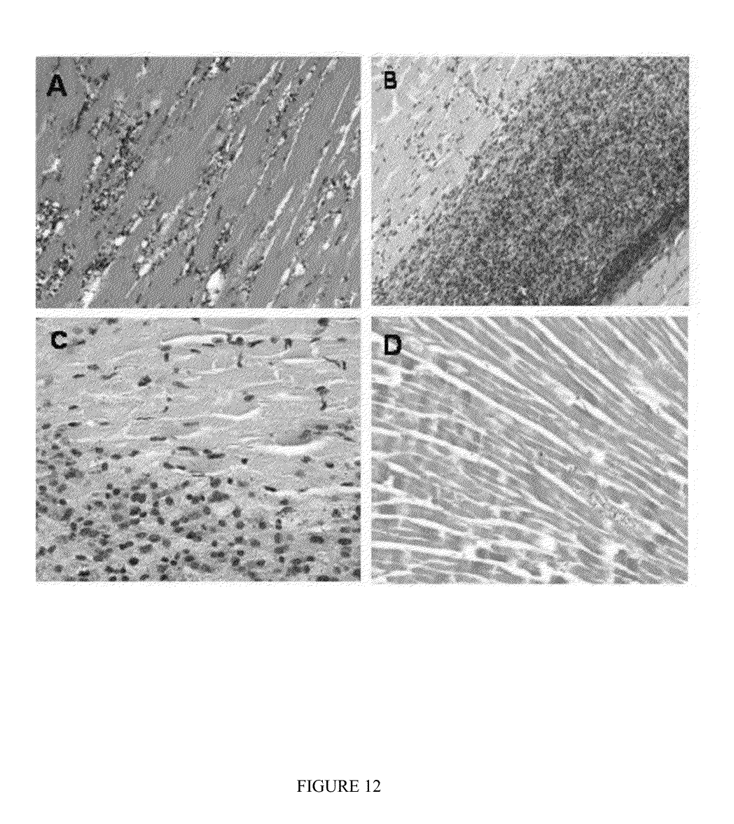

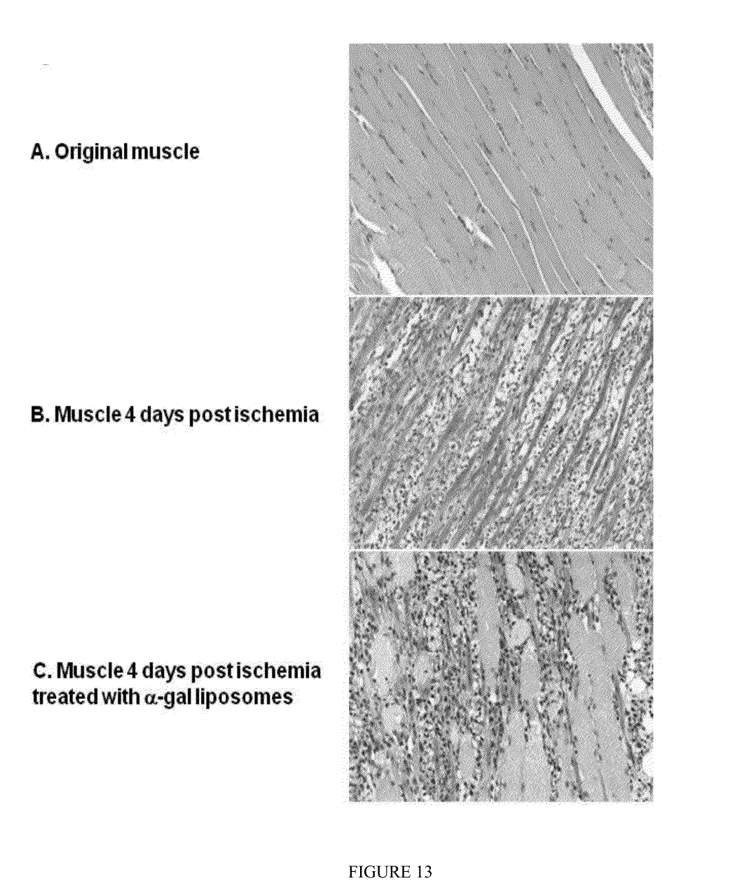

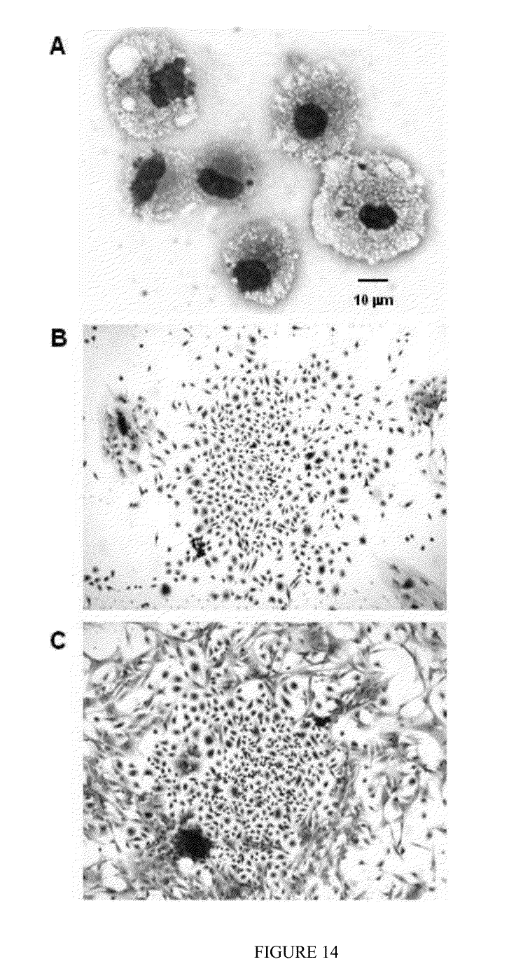

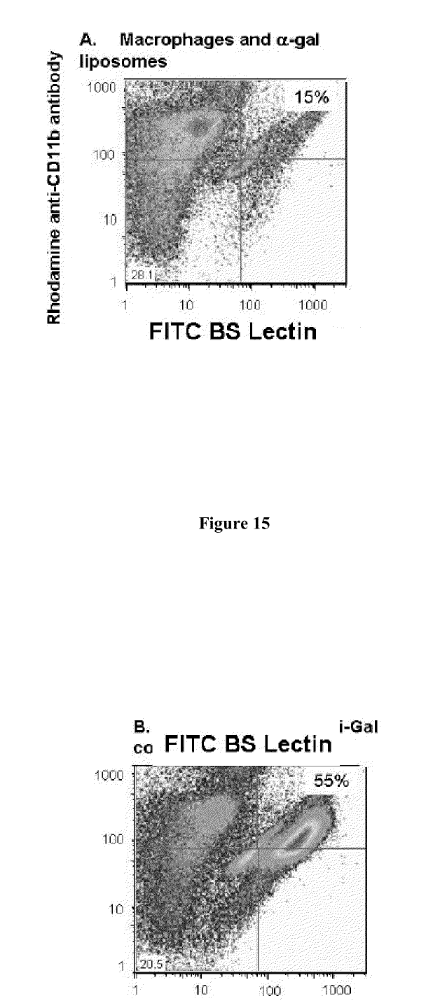

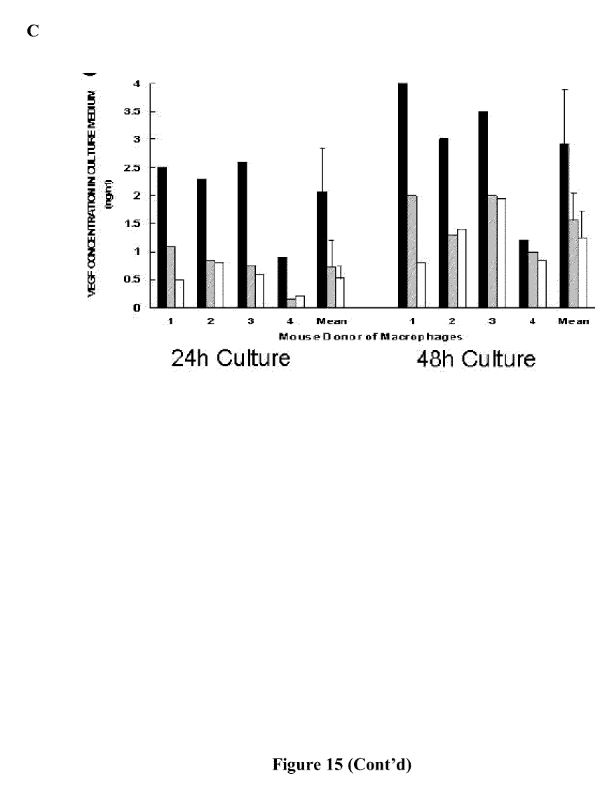

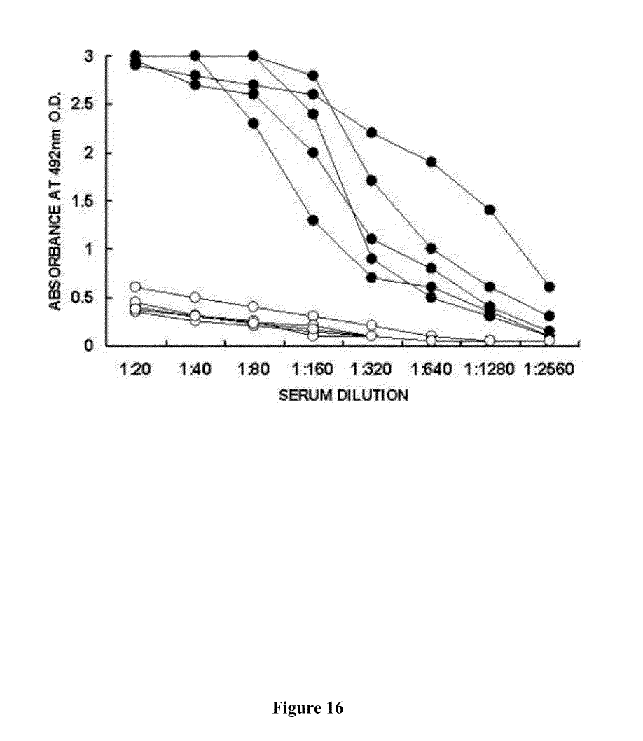

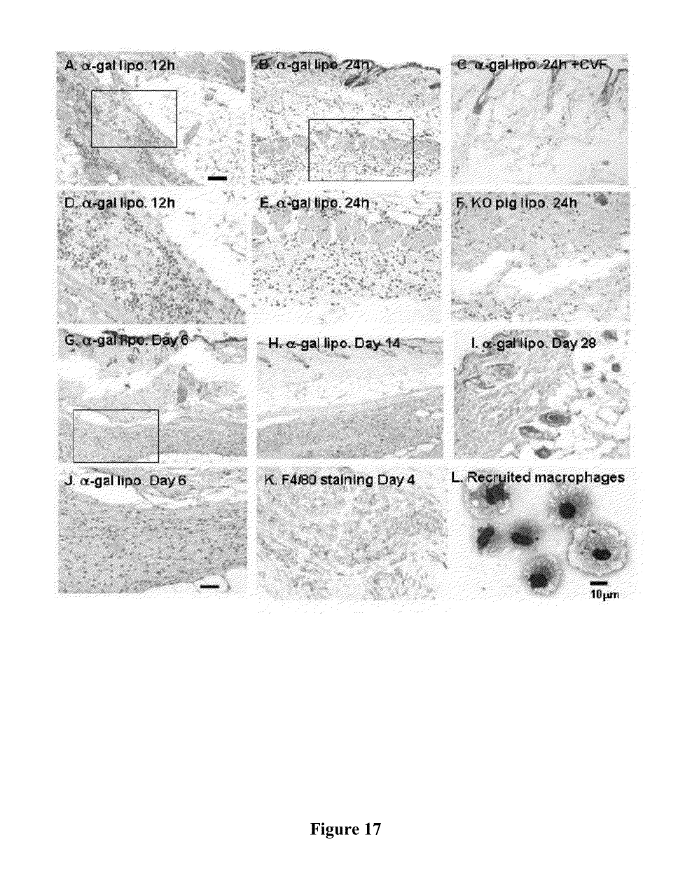

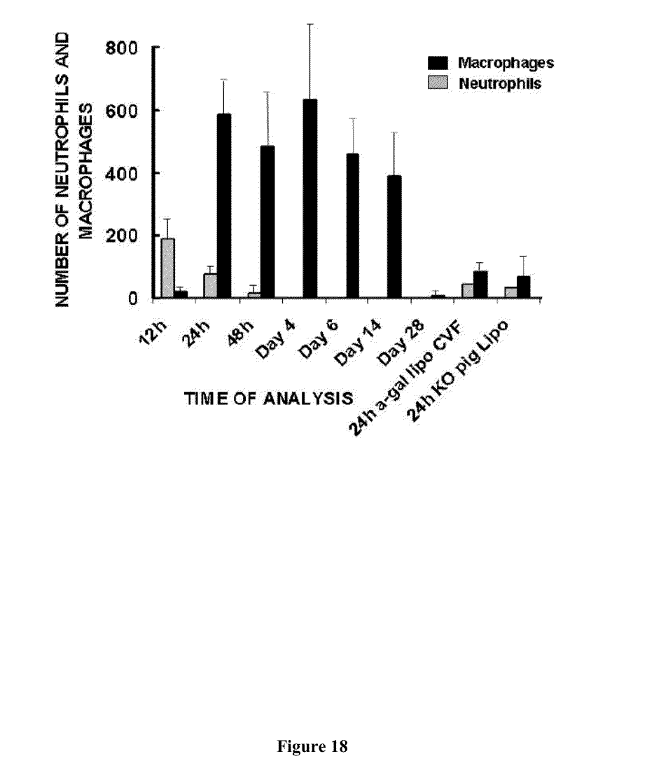

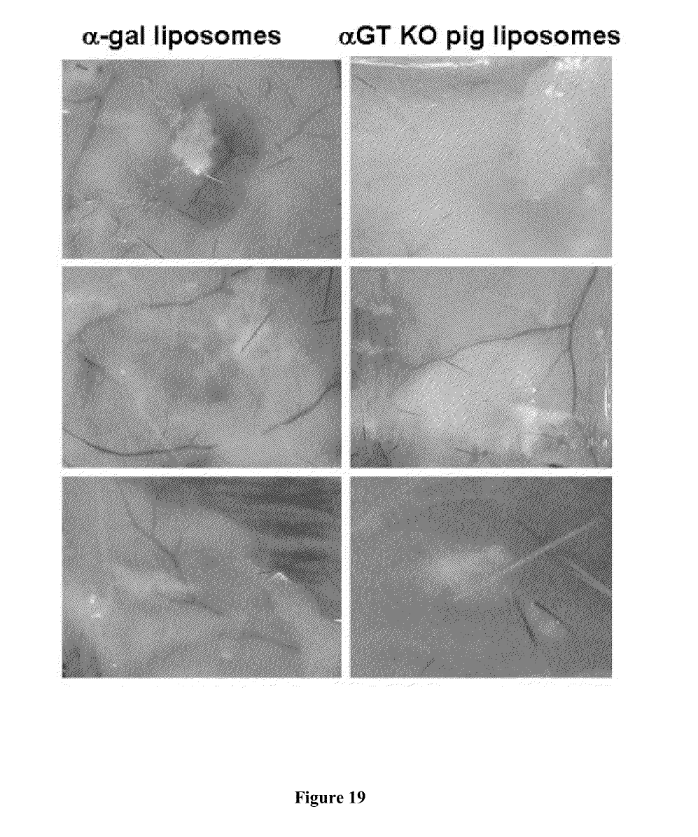

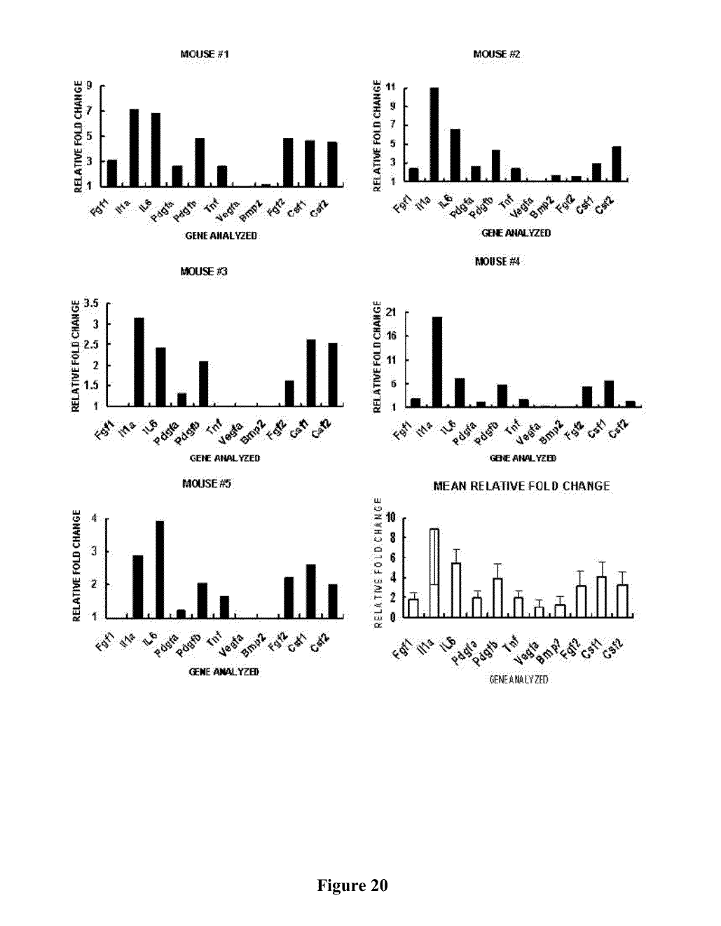

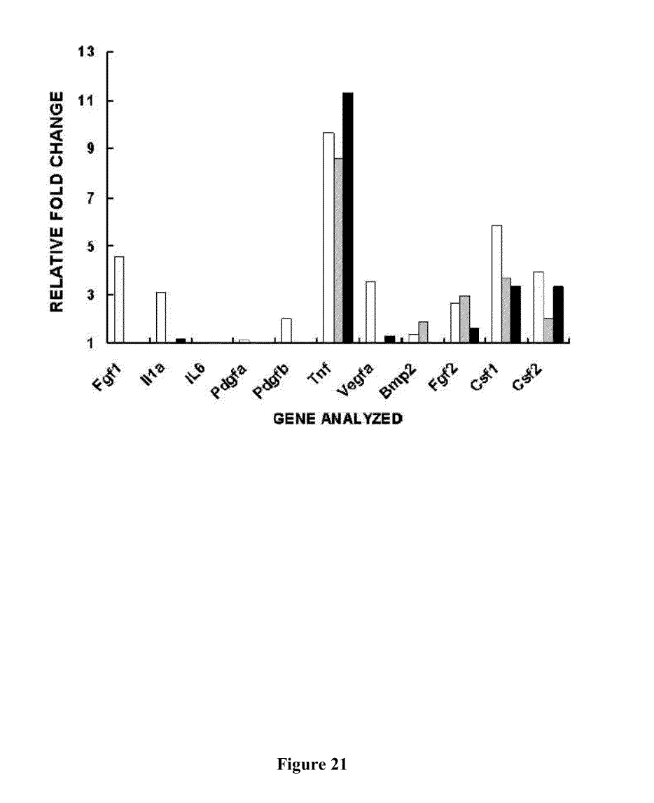

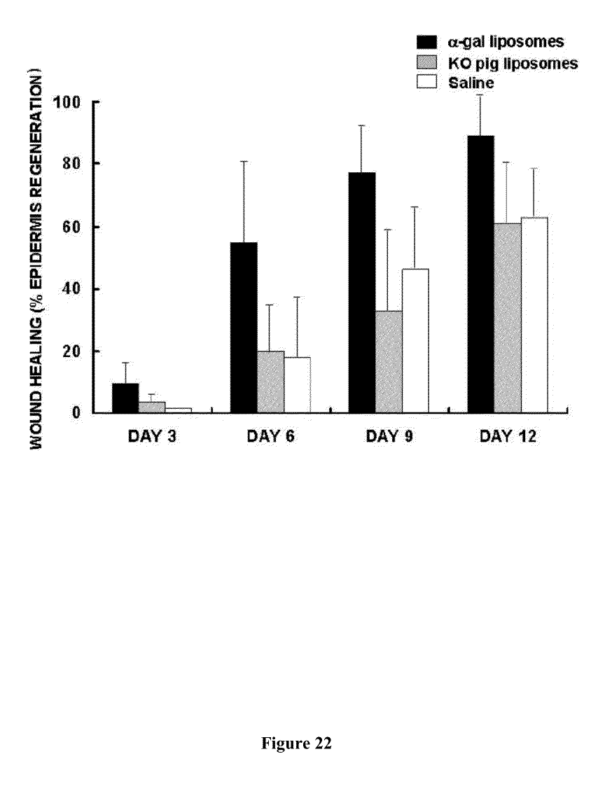

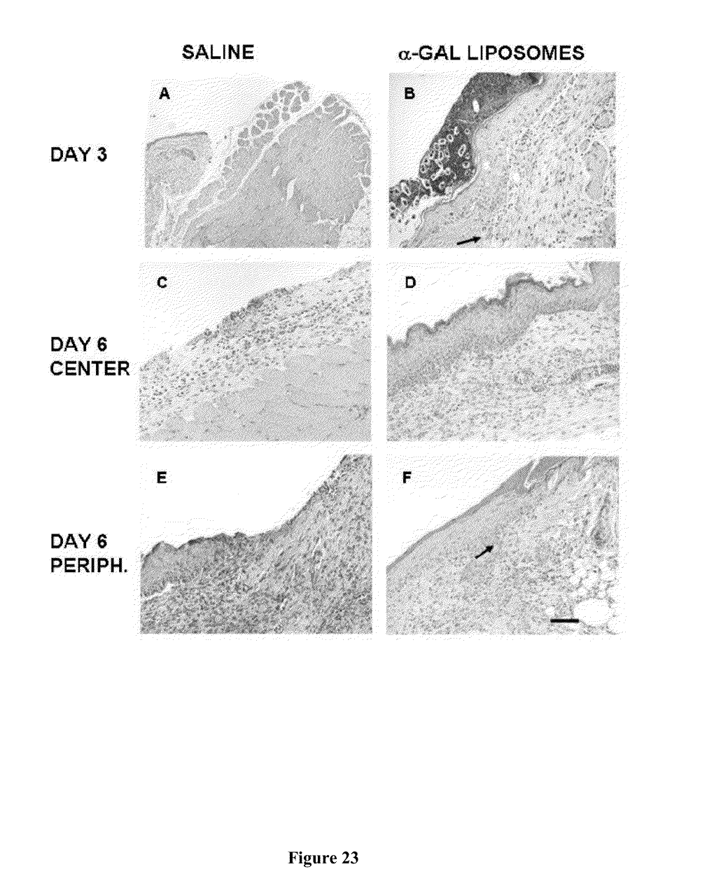

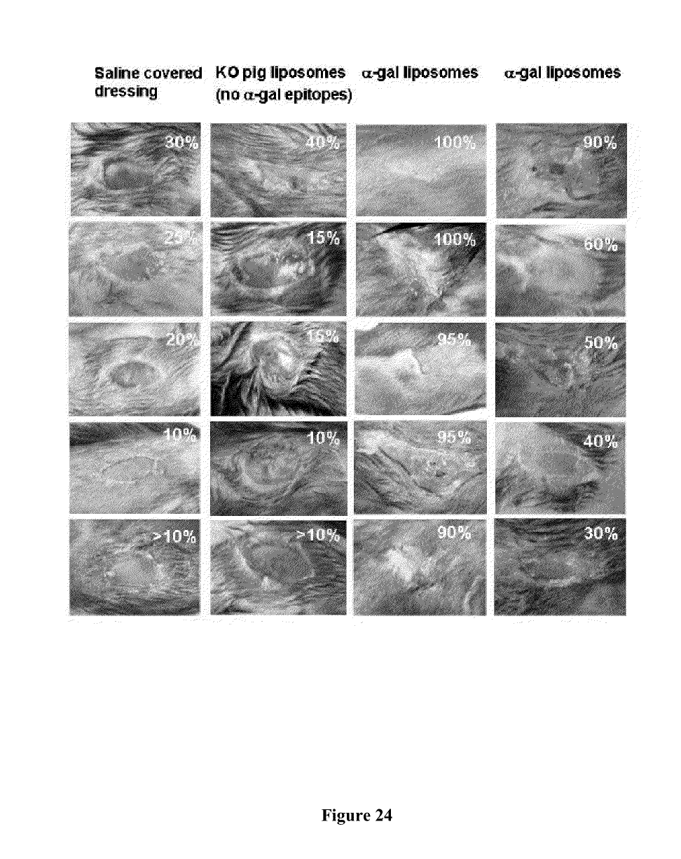

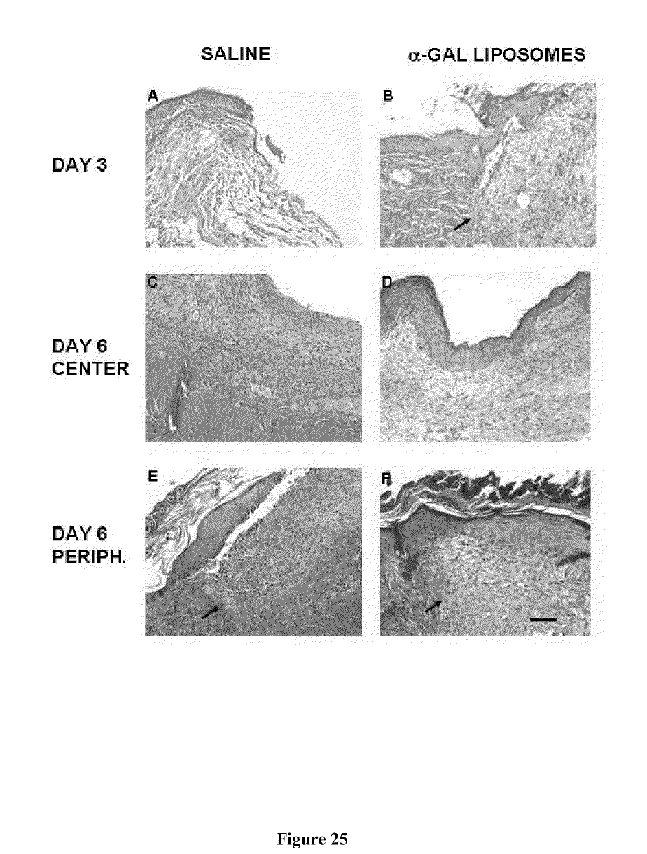

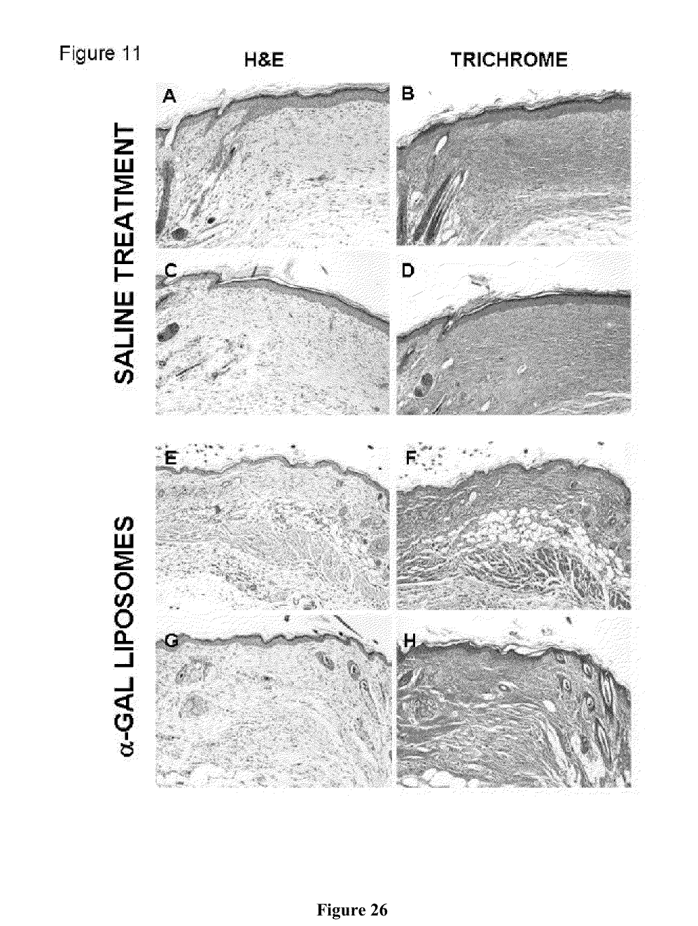

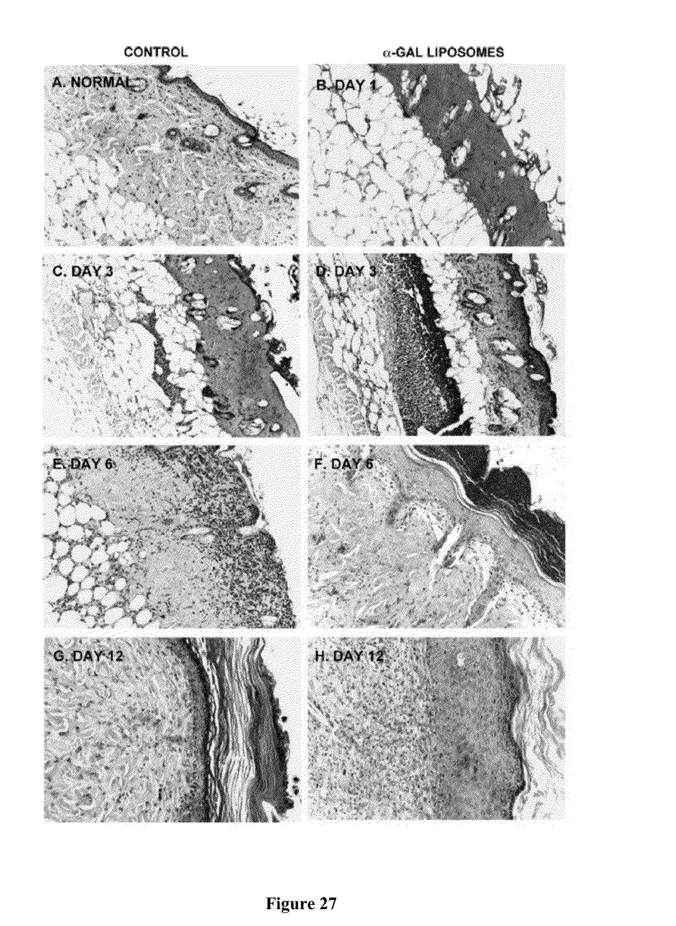

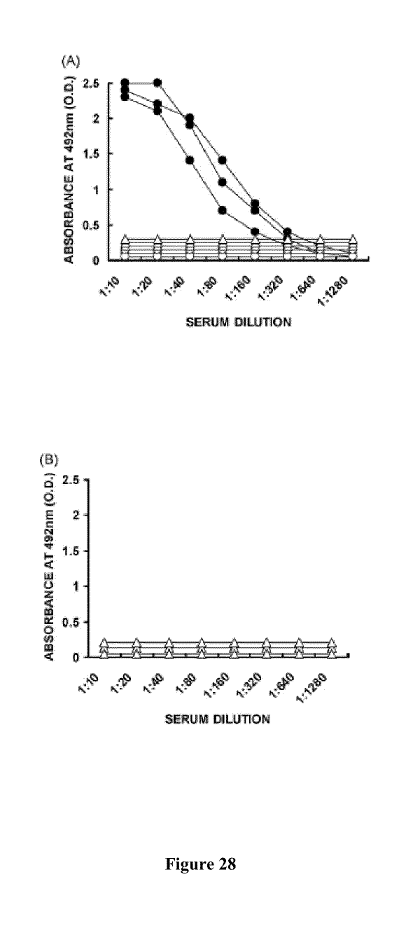

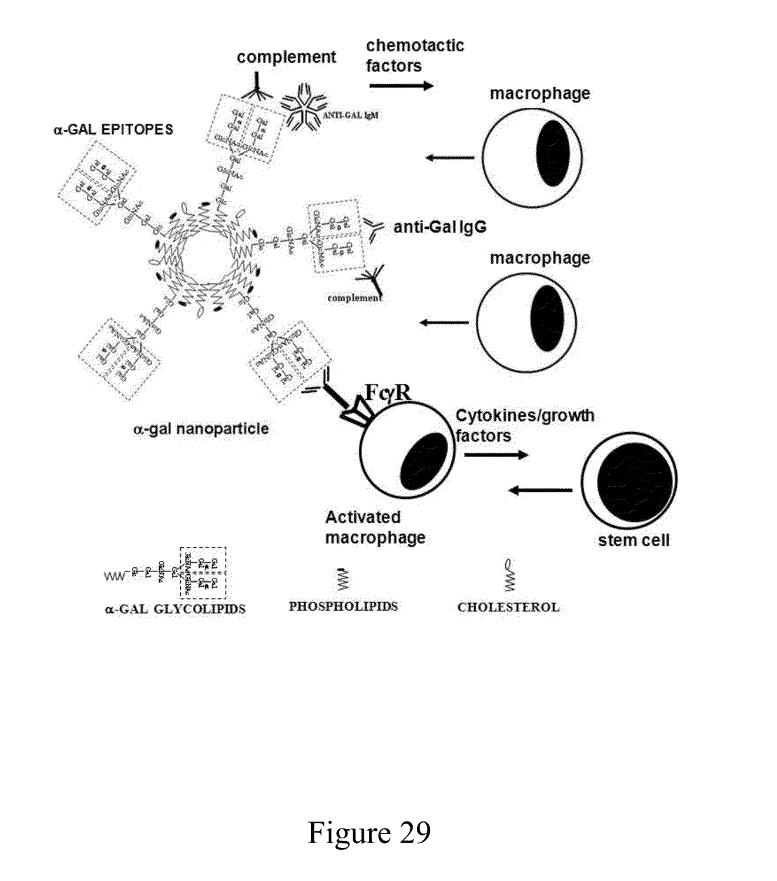

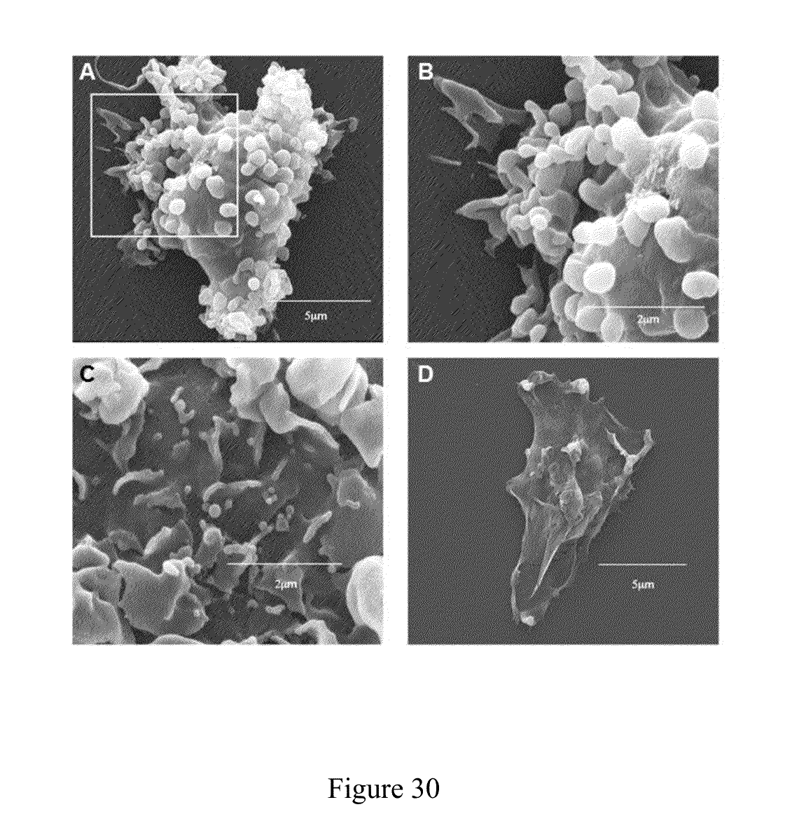

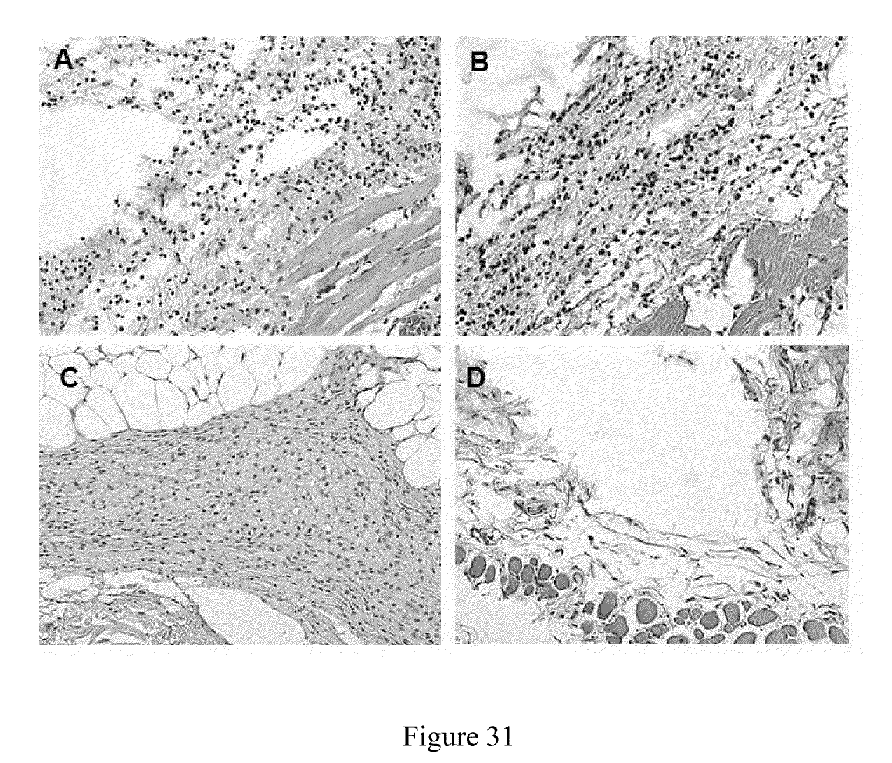

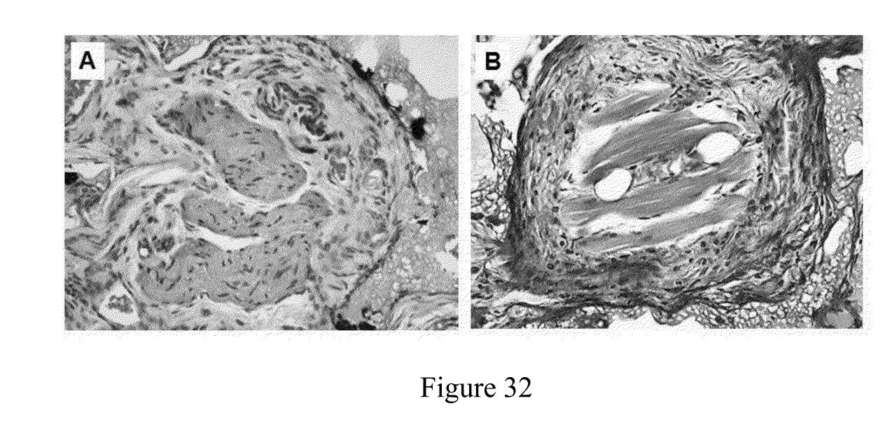

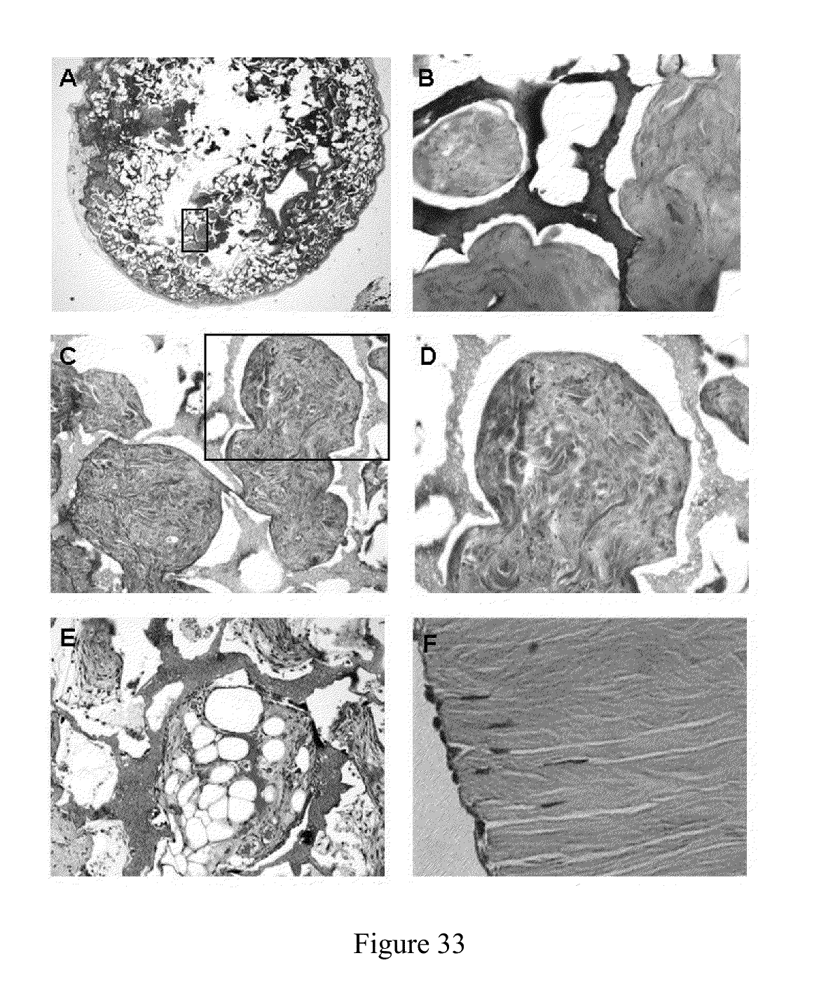

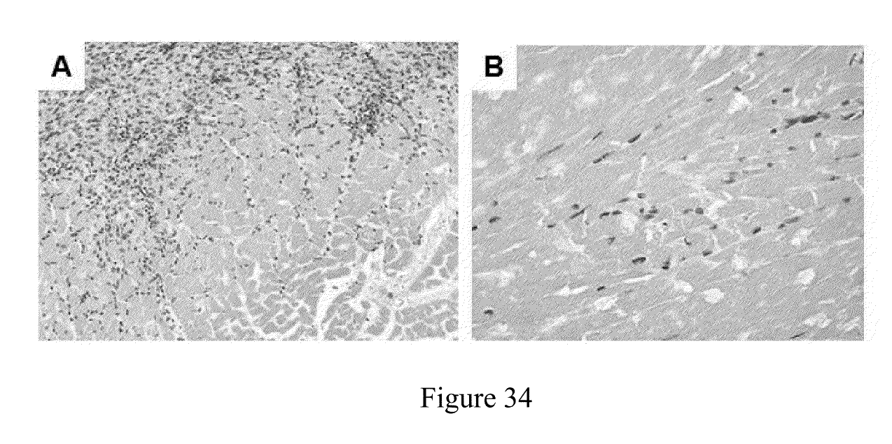

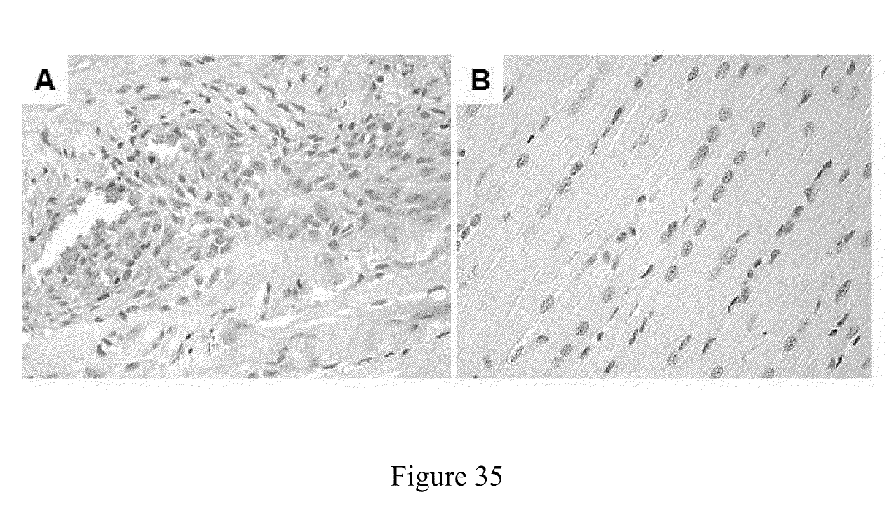

The interaction between the anti-Gal antibody and α-gal epitopes in α-gal ointment is demonstrated. The α-gal ointment cannot be used as solid phase antigen in ELISA since it does not attach to ELISA wells. Thus, the accessibility of α-gal epitopes within the ointment to anti-Gal binding was tested by mixing of the monoclonal anti-Gal M86 antibody as provide for in Galili et al., Transplantation 65, 1129, 1998, hereby incorporated by reference, with the ointment at a 1:1 ratio (vol/vol) for 1 h at 37° C. Interaction of anti-Gal with α-gal epitopes in the ointment prevents (neutralizes) subsequent binding of the monoclonal anti-Gal antibody to α-gal epitopes on the synthetic α-gal epitopes linked to bovine serum albumin (α-gal BSA), which serves as solid phase antigen in ELISA. This provides a readout system for non-neutralized anti-Gal remaining active. Mixing the antibody preparations with Vaseline served as control for lack of α-gal epitopes, i.e. no binding of anti-Gal. α-Gal ointment neutralized >95% of the monoclonal anti-Gal M86 antibody mixed with the ointment as shown in FIG. 10. In the absence of α-gal glycolipids, Vaseline had no neutralizing effect on anti-Gal. This implies that anti-Gal in burn areas will readily bind to α-gal epitopes in α-gal ointment that is applied topically.

### Example 9

### Effect of α-Gal Ointment on Burn Healing Following Thermal Injury

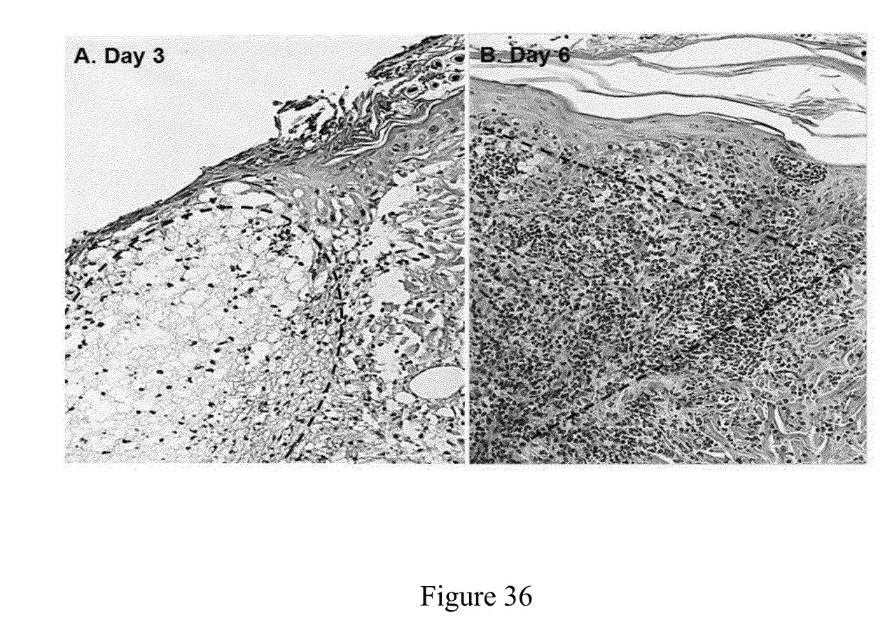

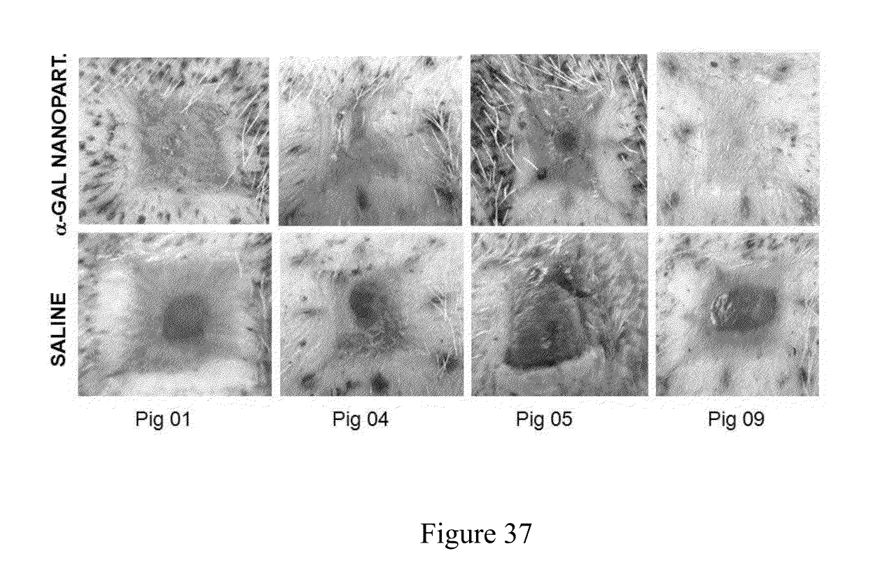

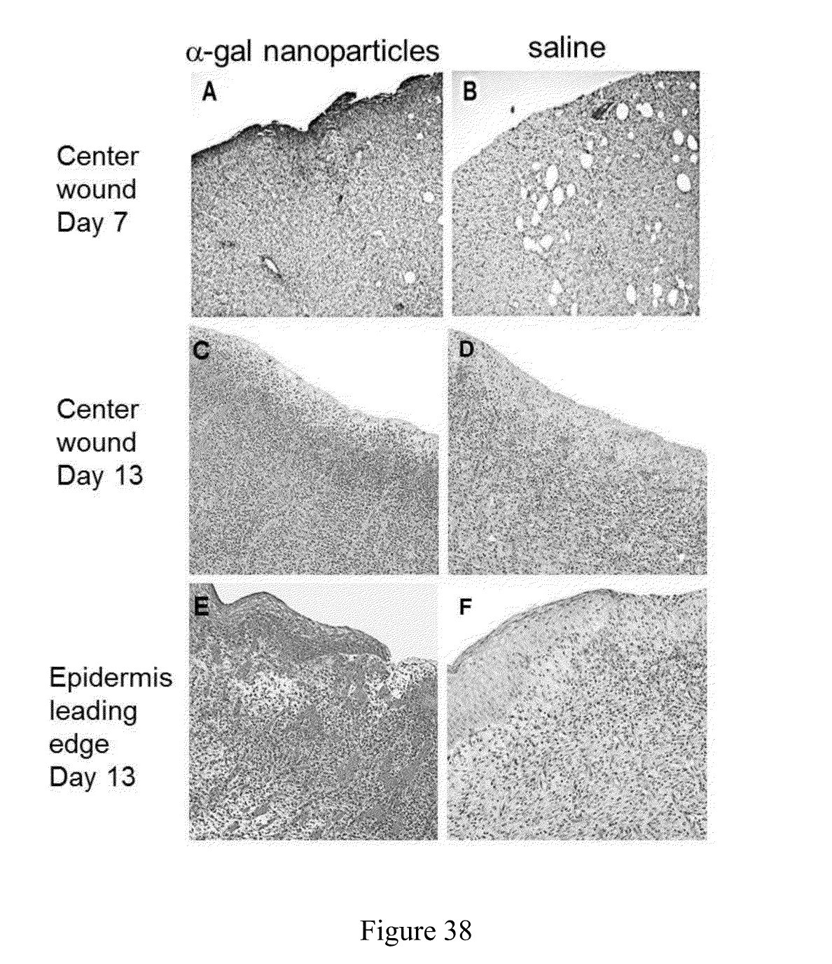

This section demonstrates the effects of α-gal ointment on healing of burns in α1,3galactosyltransferase knockout mice (KO mice) producing the anti-Gal antibody. KO mice were deeply anaesthetized with ketamine/xylazine injection and a superficial skin burn was caused in two sites on the back by brief touch with a heated end of a small metal spatula bend in the end (5 mm from the tip). Subsequently, α-gal ointment (FIG. 9) was applied topically to the right burn, whereas the left burn was covered with Vaseline lacking α-gal glycolipids. The left burn served as a control for healing of the burn in the absence of anti-Gal interaction with α-gal epitopes. The wounds were covered with circular band aids. The mice (n=4) were euthanized on Day Six, the skin areas in the burn regions inspected and removed. The skin specimens were fixed with formalin and subjected to histological sections and hematoxylin-eosin (H&E) staining (FIG. 11).

The burns were of the same size when formed by the heated edge of the metal spatula. However, after six days post-burn, the size of the damaged area treated by topical application of α-gal ointment was approximately half the size of the control wound treated with Vaseline (FIG. 11A). Histological analysis of the control Vaseline covered burns revealed the absence of the epithelial cells of the epidermis (FIG. 11B). The presence of the debris comprised of dead tissue and granulocytes (eschar) is evident as dark fragments above the injured skin. A similar eschar is observed over the burn covered with α-gal ointment (FIG. 11C). However, the skin treated with α-gal ointment was completely covered at the burn area by a new epidermis consisting of several layers of epithelial cells, as well as a keratinous layer over this epithelial layer (stratum corneum) (FIG. 11C). These findings indicate that within a period of six days, the topical application of α-gal ointment results in complete regeneration of the top layer of the skin and the formation of an epidermis barrier that seals off the dermis from any microbial agent. It should be noted that at this early stage of burn healing, no skin appendages (e.g. hair shafts or sweat glands) are observed as yet. Overall, these findings imply that the histological analysis fits the gross morphology findings of accelerated healing of burns treated with α-gal ointment.

### Example 10

### Effects of α-Gal Liposome/Anti-Gal Antibody Application on Regeneration and Repair of Damaged Cartilage in Subjects with Osteoarthritis

This example is aimed to study the efficacy of the compositions and methods of the present invention in recruitment of mesenchimal stem cells, or stem cells from any origin, for the healing and repair of damaged or injured tissues. In this example α-gal liposomes are injected into either the synovial cavity or the cartilage of human subjects having damaged articular cartilage in the joints, including but in no way limited to the knee joints of subjects with osteoarthritis. The α-gal liposomes are injected at any volume that is suitable for injection into the synovial cavity, with a preferred concentration ranging from 0.001 and 500 mg/ml. The injection is given once or several times in interval of one to several weeks. The anti-Gal antibody interaction with α-gal epitopes on α-gal liposomes results in activation of complement and local production of the complement fragments C5a and C3a, which are potent chemotactic factors. These factors induce recruitment of neutrophils, monocytes and macrophages into the synovial cavity or into cartilage, ultimately resulting in tissue repair. The Fcγ receptors on macrophages bind the Fc portion of anti-Gal coating the α-gal liposomes due to anti-Gal binding to α-gal epitopes on these liposomes. This Fc/Fcγ receptor interaction generates a signal that activates the macrophages recruited by the C5a and C3a chemotactic factors. Activated macrophages mediate debridement of the damaged cartilage and through secretion of growth factors and cytokines direct migration of stem cells that differentiate locally into chondroblasts in the damaged cartilage. The chondroblasts in turn secrete collagen and other cartilage matrix proteins and glycosaminoglycans, resulting in repair and remodeling of the damaged articular cartilage within the treated joint. Similarly, macrophages activated by the binding of α-gal liposome/anti-Gal antibody complexes mediate debridement of the damaged bone and through secretion of growth factors and cytokines recruit osteoclasts and osteoblasts into the injection site for repair and remodeling of the damaged bone. By analogy, similar injection of α-gal liposomes into damaged heart tissue (myocardium) will result in local recruitment of monocytes/macrophages into the injection site and the subsequent secretion of growth factors and cytokines by these cells recruited into the injection sites. These growth factors and cytokines direct the migration of stem cells, either from the adjacent tissue or from another source, into the damaged tissue and further direct the subsequent repair and remodeling of the damaged heart tissue. Similarly, injection of α-gal liposomes into other damaged or injured tissues in the body may result in accelerated repair of the injury by recruitment of stem cells by a mechanism similar to that described above for the damaged articular cartilage treated with α-gal liposomes.

### Example 11

### In Vivo Recruitment of Macrophages by α-Gal Liposomes Injected into Ischemic Heart Muscle

This example demonstrates the ability of α-gal liposomes to recruit macrophages into the heart muscle. Hearts removed from KO mice were injected into the myocardium with 2 mg α-gal liposomes or with saline. Subsequently, the hearts were implanted subcutaneously in KO mice producing anti-Gal. Implanted hearts injected with saline and removed after 2 weeks contained necrotic cardiomyocytes and infiltrating neutrophils (FIG. 12A). After 4 weeks the heart implants disappeared due to the destruction of the organ. In contrast, myocardium specimens from implanted hearts that were injected with α-gal liposomes tissue maintained normal histological structure for 2 and 4 weeks and contained many recruited macrophages (FIGS. 12B and 12C). In addition, many of the recruited cells migrate into areas between the dead cardiomyocytes (FIG. 12C). All the nuclei visible in the sections are those of the infiltrating cells. This is indicated in FIG. 12D which describes an inner portion of the myocardium which lacks infiltrating cells. As seen in FIG. 12D (2 weeks post implantation of an α-gal liposomes injected KO mouse heart,) no nuclei are visible in the dead cardiomyocytes. Moreover, the myocardium in α-gal liposomes treated mice maintains its histological characteristics much better than saline injected hearts (FIGS. 12B-D, vs. FIG. 12A).

### Example 12

### In Vivo Recruitment of Macrophages by α-Gal Liposomes into Ischemic Skeletal Muscle

Another example for the in vivo recruitment of macrophages by α-gal liposomes is the injection of these liposomes into a KO mouse leg muscle by ischemia. The blood flow was blocked in the right hind leg of KO mice by applying a rubber band tourniquet over the leg according to a method previously described by Ott et al., FASEB J. 19:106 (2005). The tourniquet was removed after 4 h to allow for reperfusion of the leg blood vessels. The histology studies are performed in the leg muscle (hind limb). The muscle fibers in an uninjured skeletal muscle comprise of muscle cell syncitia (myotubes), formed by fusion of myoblasts, with the nuclei in the periphery of the tubes. See, FIG. 13A. This ischemia results in death of the myotubes due to lack of oxygen. The resulting necrosis of the myotubes is clearly evident after 96 h. See, FIG. 13B. The specimen in FIG. 13B was injected with saline to serve as control to α-gal liposomes injection. At that time, many neutrophils infiltrate the necrotic tissue. The myotube syncitia decrease in their size and the nuclei of each myotube accumulate in a row. Subsequently, the dead myotubes are phagocytozed by debriding macrophages. Other ischemic leg muscles were injected with 10 mg α-gal liposomes immediately after removal of the tourniquet that prevented for 4 h blood flow into the muscle. Specimens obtained 4 days post α-gal liposomes injection (FIG. 13C) indicated that the tissue contained many more macrophages than the control tissue injected with saline (FIG. 13B). Moreover, the proportion of non-necrotic myotubes in the α-gal liposomes treated ischemic muscle was much higher than that in saline injected muscle, where the large majority of the myotubes are necrotic (FIG. 13C vs. 13B respectively). These findings indicate that the injection of α-gal liposomes induces rapid recruitment of macrophages into the skeletal leg muscle and that the rapid migration of the macrophages into the injured tissue reduces the damage caused by ischemia to the muscle.

### Example 13

### In Vivo Recruitment of Stem Cells by α-Gal Liposomes

This example addresses the question of whether the population of macrophages recruited by α-gal liposomes also includes stem cells. Infiltrating macrophages were retrieved from PVA sponge discs containing 10 mg α-gal liposomes that were implanted subcutaneously for 6 days. The macrophages were retrieved by repeated squeezing of the sponge disc in phosphate buffered saline (PBS) and are presented in FIG. 14A. These cells were cultured in vitro on cover slips for 5 days in DMEM medium containing 10% fetal calf serum. Subsequently the cover slips were washed and stained with Wright staining. As shown in FIGS. 14B and 14C, cells which are recruited into PVA sponge discs containing α-gal liposomes, included, in addition to the migrating macrophages, also cells that display an extensive ability to proliferate (200-500 cells per colony formed from one cell within a period of 5 days). The frequency of these colony forming cells among cultured macrophages from PVA sponges was found to be 3-5 cells/10⁵ macrophages. This is a similar frequency as that of mesenchimal stem cells in the bone marrow reported by Eisenberg et al., Stem Cells 24:1236 (2006). The ability to proliferate (i.e. self renew) and form colonies is one of the main characteristics of stem cells. These findings indicate that the macrophages recruited by anti-Gal/α-gal liposomes interaction, include also cells that have stem cell potential.

### Example 14

### Regeneration of Injured Brain Tissue by Treatment with α-Gal Liposomes

This example is aimed to study the efficacy of the compositions and methods of the present invention in recruitment of stem cells, for the healing and repair of damaged or injured brain tissue. In this example α-gal liposomes are injected intracranial into areas in the brain of human subjects having damage such as, but not limited to ischemia following infarct in one or more of the blood vessels in the brain. The α-gal liposomes are injected at any volume that is suitable for injection into the injured brain tissue and at a concentration ranging between 0.001 and 500 mg/ml. The interaction between the injected α-gal liposomes and the anti-Gal antibody activates complement and the generated chemotactic complement cleavage peptides which are chemotactic factors recruit monocytes and macrophages to the injection site. The recruited macrophages are activated by Fc/FcγR interaction with anti-Gal coated α-gal liposomes and secrete cytokines and growth factors that promote healing of the injured brain tissue and recruit stem cells. These stem cells proliferate and differentiate in to brain cells that repair and regenerate the injured brain tissue.

### Example 15

### Regeneration of Injured Peripheral Nerve or Injured Spinal Cord by Treatment with α-Gal Liposomes

This example is aimed to study the efficacy of the compositions and methods of the present invention in recruitment of stem cells, for the healing and repair of damaged or injured peripheral nerve or spinal cord. In this example α-gal liposomes at a concentration ranging between 0.001 and 500 mg/ml are administered into the injured spinal cord or to the injured nerve by injection or by any other method known to those skilled in the art. An alternative approach is that the injured spinal cord or peripheral nerve is surrounded by a device containing α-gal liposomes at a concentration ranging between 0.001 and 500 mg/ml. This device can be in the form of a gel, plasma or fibrin clot surrounding part or the whole injured nerve tissue or spinal cord. Alternatively, collagen sheet or any biodegradable or non-biodegradable sheet containing the α-gal liposomes or having on its surface α-gal liposomes and which can be shaped into a tube around the injured nerve or spinal cord, can be used to apply the α-gal liposomes around the injured nerve or the injured spinal cord. The interaction between the injected α-gal liposomes and the anti-Gal antibody activates complement and the generated chemotactic complement cleavage peptides which are chemotactic factors that recruit monocytes and macrophages to the injection site. The macrophages are activated by Fc/FcγR interaction with anti-Gal coated α-gal liposomes and secrete cytokines and growth factors that promote extension of the damaged axons for reconnecting with the distal portion of the damaged neurons and growing into the distal portion of the axonal tube. Alternatively, the stem cells recruited by these cytokines and growth factors proliferate and differentiate in to nerve cells that promote regeneration of the injured nerve tissue in the peripheral nerve and/or in the spinal cord. Injection of α-gal liposomes into the retina, lens, or cornea of the eye could be beneficial in the recruitment of stem cells that repair damages in these tissues of the eye.

### Example 16

### Treatment of Diabetic Patients by Injection of α-Gal Liposomes into the Pancreas

This example is aimed to study the efficacy of the compositions and methods of the present invention in recruitment of stem cells, for regenerating the activity of Langerhans Islets in the pancreas of diabetic patients. In patients with Type I diabetes and in some of the patients with Type II diabetes the Langerhans Islets have been destroyed. The proposed treatment aims to restore biologically active Langerhans Islets in the pancreas of these patients, thereby provide endogenous insulin and cure the state of diabetes. In this example, α-gal liposomes at a concentration ranging between 0.001 and 500 mg/ml are injected into the pancreas by endoscopy ultrasound, or by laparoscopy or by any other procedure which enables for direct injection of the α-gal liposomes into the pancreas. The interaction between the injected α-gal liposomes and the anti-Gal antibody activates complement and the generated chemotactic complement cleavage peptides which are chemotactic factors that recruit monocytes and macrophages to the injection site. The macrophages are activated by Fc/FcγR interaction with anti-Gal coated α-gal liposomes and secrete cytokines and growth factors that recruit stem cells. These stem cells and/or stem cells originating from macrophages proliferate and differentiate into Langerhans Islet cells that form the islets and secrete endogenous insulin.

### Example 17

### Treatment of Patients with Injuries in the Gastrointestinal Track By Injection of α-Gal Liposomes

This example is aimed to study the efficacy of the compositions and methods of the present invention in recruitment of stem cells, for repair and regeneration of the gastrointestinal wall in patients with ulcer and other injuries to the gastrointestinal tract. The non-limiting example here is of ulcers in the stomach. This described treatment is applicable to any damage to the wall at any part of the gastrointestinal tract. The injured area is injected with α-gal liposomes at a concentration ranging between 0.001 and 500 mg/ml. The interaction between the injected α-gal liposomes and the anti-Gal antibody activates complement and the generated chemotactic complement cleavage peptides which are chemotactic factors that recruit monocytes and macrophages to the injection site. The macrophages are activated by Fc/FcγR interaction with anti-Gal coated α-gal liposomes and secrete cytokines and growth factors that recruit stem cells and promote the repair of the injured tissue. The recruited stem cells proliferate and differentiate into cells that replace the injured cells and repair the damaged gastrointestinal wall at the injection site.

### Example 18

### Treatment of Patients with Injuries Blood Vessels by α-Gal Liposomes

This example is aimed to study the efficacy of the compositions and methods of the present invention in recruitment of stem cells, for repair and regeneration of the blood vessel wall in patients with damaged blood vessels or in anastomoses of blood vessels by the use of α-gal liposomes. The injured blood vessel is surrounded by a device containing α-gal liposomes at a concentration ranging between 0.001 and 500 mg/ml. This device can be in the form of a gel, plasma clot or fibrin clot surrounding part or the whole injured blood vessel. Alternatively, collagen sheet or any biodegradable or non-biodegradable sheet containing the α-gal liposomes or having on its surface α-gal liposomes and which can be shaped into a tube around the injured blood vessel can be used to apply α-gal liposomes around the injured blood vessel. The interaction between the injected α-gal liposomes and the anti-Gal antibody activates complement and the generated chemotactic complement cleavage peptides which are chemotactic factors that recruit monocytes and macrophages to the injection site. The macrophages are activated by Fc/FcγR interaction with anti-Gal coated α-gal liposomes and secrete cytokines and growth factors that promote the repair of the injured blood vessel wall. These secreted cytokines and growth factors also recruit stem cells that proliferate and differentiate into cells that enable the regeneration of the intact blood vessel wall. Some of the recruited macrophages, which have stem cell potential, also may trans-differentiate into cells that repair the injured blood vessel.

### Example 19

### Wound Healing Using Topically Applicants α-Gal Liposomes

### Materials

Rabbit RBC and pig kidneys were purchased from PelFreez (Rogers, A R). Pig RBC from α1,3galactosyltransferase knockout pigs (KO pigs) were a generous gift from Fios Therapeutics. Peroxidase (HRP) coupled goat anti-mouse IgG and IgM antibodies were purchased from Accurate Chemicals (Westbury, N.Y.), HRP coupled F4/80 anti-mouse antibody from Caltag (Invitrogen, MD) and rhodamin coupled antibodies for CD11b from Pharmingen (San Diego, Calif.). HRP coupled rabbit anti-human IgG antibodies were purchased from Dako (Copenhagen, Denmark). FITC coupled Bandeiraea (Griffonia) simplicifolia IB4 lectin (BS lectin) was purchased from Vector Labs (Burlingame, Calif.). Cobra venom factor (CVF) was purchased from Sigma (St. Louis, Mo.).

### Preparation of α-Gal Liposomes and Liposome Wound Dressings

α-gal glycolipids comprise the majority of glycolipids in these RBC. Galili et al., 2007 “Intratumoral injection of α-gal glycolipids induces xenograft-like destruction and conversion of lesions into endogenous vaccines” J. Immunol. 178:4676-4687; Eto et al., 1968 “Chemistry of lipids of the posthemolytic residue or stroma of erythrocytes. XVI. Occurrence of ceramide pentasaccharide in the membrane of erythrocytes and reticulocytes in rabbit” J. Biochem. (Tokyo) 64:205-213; Stellner et al., 1973 “Determination of aminosugar linkage in glycolipids by methylation. Aminosugar linkage of ceramide pentasaccharides of rabbit erythrocytes and of Forssman antigen” Arch. Biochem. Biophys. 133: 464-472; Dabrowski et al., 1984 “Immunochemistry of I/i-active oligo- and polyglycosylceramides from rabbit erythrocyte membranes. Determination of branching patterns of a ceramide pentadecasaccharide by 1H nuclear magnetic resonance” J. Biol. Chem. 259:7648-7651; and Egge et al., 1985 “Immunochemistry of I/i-active oligo- and polyglycosylceramides from rabbit erythrocyte membranes. Characterization of linear, di-, and triantennary neolactoglycosphingolipids” J. Biol. Chem. 260: 4927-4935; Hanfland et al., 1988 “Structure elucidation of blood group B-like and I-active ceramide eicosa- and pentacosasaccharides from rabbit erythrocyte membranes by combined gas chromatography-mass spectrometry; electron-impact and fast-atom-bombardment mass spectrometry; and two-dimensional correlated, relayed-coherence transfer, and nuclear Overhauser effect 500-MHz 1H-n.m.r. spectroscopy” Carbohydr. Res. 178:1-21; and Honma et al., 1981 “Isolation and partial structural characterization of macroglycolipid from rabbit erythrocyte membranes” J. Biochem. (Tokyo). 90:1187-1196. Therefore, α-gal liposomes were prepared from rabbit RBC membranes. Galili et al., 2010 “Accelerated healing of skin burns by anti-Gal/α-gal liposomes interaction” Burns 36:239-251. Batches of 1 liter rabbit RBC were lysed in water and washed repeatedly to remove hemoglobin. For the extraction process, rabbit RBC membranes (RBC ghosts) were mixed with 1000 ml chloroform and 1000 ml methanol (1:1 chloroform:methanol) for 2 h, then 1000 ml methanol was added for overnight incubation with constant stirring (1:2 chloroform:methanol). The extract was filtered under vacuum through Whatman filter paper for removing residual RBC membranes and precipitated proteins. The membrane extract was dried in a rotary evaporator, and sonicated in saline in a sonication bath. The liposomes are spun at 1000 rpm for 10 min to remove precipitating materials which form a pellet. Supernatants containing liposomes were further centrifuged at 14,000 rpm and liposome pellets resuspended in the saline supernatant at a final concentration 100 mg/ml (10% vol/vol). These liposomes were extensively sonicated for 10 min using a sonication probe on ice within a laminar flow hood. The liposomes are referred to as α-gal liposomes as they present an abundance of α-gal epitopes on their membranes.

### Preparation of Control Liposomes Lacking α-Gal Epitopes

Control liposomes lacking α-gal epitopes were prepared from α1,3galactosyltransferase knockout pigs (KO pig) RBC. These αGT KO pigs lack α-gal epitopes because of targeted disruption (knockout) of the α1,3GT gene. Byrne et al., 2008 “Proteomic identification of non-Gal antibody targets after pig-to-primate cardiac xenotransplantation” Xenotransplantation 15:268-276. The KO pig RBC were received as a generous gift from Fios Therapeutics (Rochester, Minn.). Control KO pig liposomes were prepared by a method identical to the one described above for α-gal liposomes.

### Breeding and Immunization of α1,3Galactosyltransferase Knockout Mice

Mice used in this study have disrupted (e.g., knockout) α1,3GT genes and are referred to as αGT knockout (KO) mice. Thall et al., 1995 “Oocyte Gal α1,3Gal epitopes implicated in sperm adhesion to the zona pellucida glycoprotein ZP3 are not required for fertilization in the mouse” J. Biol. Chem. 270:21437-21440. The mice were generated in C57BL/6 genetic background and are bred and maintained at the animal facility of the University of Massachusetts Medical School. All experiments were performed with both male and female mice. Study protocols were approved by the UMass IACUC and are in compliance with national guidelines. Anti-Gal antibody production was elicited in KO mice by 3-4 weekly i.p. immunizations with 50 mg pig kidney membrane (PKM) homogenate, i.e. xenogeneic membranes expressing multiple α-gal epitopes. Production of anti-Gal antibody in KO mice was confirmed to be at titers similar to those observed in humans (i.e., for example, titers of between approximately 1:100 to 1:2000), by ELISA with α-gal BSA as solid phase antigen. Tanemura et al., 2000 “Differential immune responses to α-gal epitopes on xenografts and allografts: implications for accommodation in xenotransplantation” J. Clin. Invest. 105:301-310; Abdel-Motal et al., 2006 “Increased immunogenicity of HIV gp120 engineered to express α-gal epitopes” J. Virol. 80:6943-6951; and Abdel-Motal et al., 2009 “Mechanism for increased immunogenicity of vaccines that form in vivo immune complexes with the natural anti-Gal antibody” Vaccine 27:3072-3082.

Treatment of Excisional Skin Wounds with α-Gal Liposomes

Wounds were formed in shaved abdominal flanks of anesthetized KO mice. A 3×6 mm oval skin incision was made in the right abdominal flank of the mouse. The epidermis, dermis and upper part of the hypodermis were removed in the wound area created by this incision, resulting in the exposure of the connective tissue fascia over the panniculus carnosus muscle layer. Prior to treatment, 0.1 ml of the liposome suspension containing 10 mg α-gal liposomes was applied onto the pad (1×1 cm) of a small circular wound dressing (“spot” bandage, CVS Pharmacies) in a sterile laminar flow hood. The pads of the control wound dressings had either 0.1 ml saline or 10 mg KO pig liposomes applied. The wound dressing was applied to cover the wound and was further covered with Tegaderm™ and with Transpore™ adhesive tape (3M, St. Paul, Minn.) in order to prevent removal by the mouse.

### Example 20

### Preparation of Peritoneal Macrophages

KO mice were injected intraperitoneally (i.p.) with 1.5 ml of a 4% Brewer's thioglycolate solution. Macrophages (>99%) migrating into the peritoneal cavity were harvested after 7 days by i.p. injection of 10 ml PBS into euthanized mice and subsequent collection of the fluid from the peritoneal cavity.

Binding of anti-Gal antibody coated α-gal liposomes via Fc/FcγR interaction in macrophages was measured by flow cytometry. α-gal liposomes were coated with mouse anti-Gal IgG antibodies by 1 h incubation with KO mouse serum diluted 1:50. The liposomes (1 mg/ml) were washed and further incubated with mouse peritoneal macrophages for 1 h at 4° C. The cells were washed at 1000 rpm for removal of unbound liposomes then stained with rhodamin anti-CD11b antibody (macrophage specific) and with FITC-Bandeiraea (Griffonia) simplicifolia IB4 lectin (BS lectin) which binds to α-gal epitopes on the liposomes. After 30 min incubation, cells were washed, fixed and subjected to flow cytometry analysis. Macrophages incubated with non-antibody coated α-gal liposomes served as controls.

### Example 21

### Analysis of the Expression of Cytokine Genes Associated with Tissue Healing

Activation of macrophage genes encoding for cytokines associated with healing was evaluated in the skin of KO mouse skin 48 h post injection with 10 mg α-gal liposomes and in peritoneal macrophages, 24 h post i.p. injection of 30 mg α-gal liposomes.

Gene activation in the injected skin or in peritoneal macrophages was determined by quantitative real time-PCR (q-RT-PCR). Skin specimens from mice injected with saline or peritoneal macrophages from mice injected i.p. with saline served as controls in the corresponding studies. Custom made SABiosciences (Frederic, Md.) q-RT-PCR 96 well plates containing primers for 11 cytokine encoding genes and for the house keeping gene GADPH (glyceraldehydes-3-phosphate dehydrogenase) were used for this purpose. The reaction was performed with SYBR Green® master mix solution (SABiosciences PA-011).

Expression of the following genes was measured: i) Fgf1 (fibroblast growth factor 1); ii) Il1a (interleukin 1a); iii) IL6 (interleukin 6); iv) Pdgfb (platelet derived growth factor b); v) Tnf (tumor necrosis factor a); vi) Vegfa (vascular endothelial growth factor a); vii) Bmp2 (bone morphogenic protein 2); viii) Fgf2 (fibroblast growth factor 2); ix) Csf1 (colony stimulating factor 1); and x) Csf2 (colony stimulating factor 2).

Total RNA was isolated using gentle MACS (Myltenyi Extractor apparatus), followed by mRNA isolation and cDNA synthesis using Miltenyi Magnetic Micro Beads. The cDNA was added as ˜1 ng per well to wells containing the various primers. PCR reaction (30 cycles) was performed in the Biorad MyiQ single color Real Time PCR detection system. The results were normalized based on the house keeping gene and fold increase in Cₜ values (threshold concentration) determined by using the software program provided on SABioscience web site that calculates DDCₜ based fold change.

### Example 22

### Analysis of In Vitro Secretion of VEGF by Macrophages

Macrophages co-incubated with anti-Gal coated α-gal liposomes, or with α-gal liposomes not coated by anti-Gal antibody were plated in 24 well plates at 3×10⁵ cells/ml/well. Macrophages cultured without liposomes served as control. Supernatants were collected after 24 h and 48 h and subjected to analysis of VEGF secretion using VEGF ELISA kit (Antigenix, NY) according to the manufacturer's protocol.

### Example 23

### ELISA with Liposomes as Solid Phase Antigen

Binding of anti-Gal IgG in KO mouse sera to α-gal liposomes and to KO pig liposomes (control liposomes lacking α-gal epitopes) was studied in ELISA wells that were coated with these liposomes.

Liposomes in PBS (0.1 mg/ml) were dried in ELISA wells, resulting in firm attachment of the liposomes to the wells. After blocking with PBS containing 1% BSA, KO mouse serum samples at serial two-fold dilutions were placed as 50 μl aliquots in liposome coated wells and incubated for 2 h at 24° C. The wells were washed with PBS containing 0.05% Tween, and HRP coupled anti-mouse IgG antibodies added for 1 h. Color reaction was developed with ortho-phenylene diamine (OPD) and absorbance measured at 492 nm.

### Example 24

### Histological Analysis

Wound healing was determined in histological sections and expressed as percentage of wound surface covered with regenerating epidermis.

The wound bed was determined by the intact dermis. The number of infiltrating neutrophils and macrophages at skin sites injected with liposomes was determined by counting cells within a rectangular area demarcated in a microscope lens at 400× magnification. The rectangle with a size corresponding to 100×200 μm was placed at the border of the liposome hypodermic injection site within the skin. Neutrophils were identified by segmented nuclei and macrophages by the kidney or oval shaped nuclei and large size of the cells. Four fields were counted in each section. Two sections of the same specimen were evaluated. The data represent mean+SD from >5 mice/group.

### Example 25

### Statistics

A Chi² test was used for statistical analyses. A p-value<0.05 was considered statistically significant.

### Example 26

### Effect of α-Gal Liposomes on Healing of Burns in the Skin

This example demonstrates the effects of α-gal liposomes on healing of burns in the skin of α1,3galactosyltransferase knockout mice (KO mice) producing the anti-Gal antibody.

KO mice were deeply anaesthetized with ketamine/xylazine injection and a superficial skin burn was caused in two sites on the back by brief touch with a heated end of a small metal spatula bend in the end (5 mm from the tip).

The treatment was provided by topically applying 10 mg α-gal liposomes on a spot bandage pad (10×10 mm) to the right burn, whereas the left burn was covered with bandage containing saline. The left burn served as a control for healing of the burn in the absence of α-gal liposomes. The mice were euthanized on different time points, the skin areas in the burn regions inspected and removed. The skin specimens were fixed with formalin and subjected to histological sections and hematoxylin-eosin (H&E) staining for evaluating the extent of burn healing by measuring percent regeneration of epidermis. Galili et al., “Accelerated healing of skin burns by anti-Gal/α-gal liposomes interaction” Burns 36:239-251 (2010).

### Example 27

### Recruitment of Macrophages by α-Gal Nanoparticles in Skin of GT-KO Mice

In vivo studies on the effect of α-gal nanoparticles were performed in α1,3galactosyltransferase knockout (KO) mice (Thall et al., J Biol Chem, 270:21437, 1995), producing the anti-Gal antibody. The mice were induced to produce the anti-Gal 50 mg pig kidney membranes. KO mice producing anti-Gal antibody were injected subcutaneously with 10 mg of submicroscopic α-gal nanoparticles in 0.1 ml saline. Other mice were injected subcutaneously with saline

Skin specimens from the injection site were obtained at different time points, fixed, stained with hematoxylin-eosin (H&E) and inspected under a light microscope. Multiple macrophages were seen in the area of injection within 24 h due the recruitment by chemotactic complement cleavage peptides generated as a result of anti-Gal/α-gal nanoparticles interaction (FIG. 31A). The number of migrating macrophages increased after 48 h and the neutrophils disappeared (FIG. 31B). By day 7 the injection area is filled with macrophages that are very large (FIG. 31C). This morphology of macrophages as large cells is likely to be the result of the activation by Fc/FcγR interaction with anti-Gal coated α-gal nanoparticles. The infiltrating macrophages are found in the injected skin even on day 14 post injection, but they disappear after 3 weeks (not shown). Subcutaneous injection of saline resulted in no recruitment of cells in the injection site at any time point. FIG. 31D demonstrates the injection site of saline after 48 h. Overall the findings in FIG. 31 imply that submicroscopic α-gal nanoparticles injected into tissues rapidly recruit macrophages. This recruitment observed already within 24 h post injection of α-gal nanoparticles. This recruitment is temporary and lasts for 14-17 days. Subsequently the macrophages disappear from the injection site without affecting the architecture of the injection site and without causing any long-term granuloma or any detrimental other inflammatory reaction.

### Example 28

### Interaction Of Anti-Gal Coated α-Gal Nanoparticles With Fcγ Receptors on Macrophages

Recruited macrophages that reach the α-gal nanoparticles due to chemotaxis by complement cleavage chemotactic factors further interact via their Fcγ receptors (FcγR) with the Fc “tails” of anti-Gal coating α-gal nanoparticles (FIG. 29). This interaction results in generation of a trans-membrane signal that induces activation of the macrophages to produce and secrete a variety of cytokines and growth factors (referred to as cytokines/growth factors) which facilitate tissue repair and regeneration, including recruitment of stem cells. In the present example the actual binding of α-gal nanoparticles to macrophages is described in FIG. 30 where sumicroscopic α-gal nanoparticles (1 mg/ml) immunocomplexed (i.e. coated) with human natural anti-Gal antibody were incubated in tissue culture plates with cultured macrophages of α1,3GT knockout pig origin (KO pig). After 2 h incubation at room temp. the plates were extensively washed to remove nonadherent α-gal nanoparticles, macrophages were fixed and subjected to scanning electron microscopy processing and analysis. As shown in FIGS. 30A and 30B, multiple α-gal nanoparticles attach to the macrophages via the Fc/FcγR interaction. The size of these α-gal nanoparticles is 100-300 nm. In the higher magnification in FIG. 30C, binding of α-gal nanoparticles with size of 10-30 nm is demonstrated as well. If the α-gal nanoparticles are not coated with the natural anti-Gal antibody then no binding of these nanoparticles to the macrophages is observed (FIG. 30D) since in the absence of antibody on the nanoparticles, they cannot bind to the FcγR on the macrophage cell membrane.

### Example 29

### Recruitment of Macrophages into a KO Mouse Heart Muscle Containing α-Gal Nanoparticles

This invention teaches how to induce macrophage recruitment into various organs for the purpose of repair and regeneration. In one embodiment such recruitment of macrophages may be induced in ischemic myocardium following myocardial infarction in order to initiate a regeneration process of the injured myocardium and thus, to avoid fibrosis and scar formation. In another embodiment, inclusion of α-gal nanoparticles in decellularized organs or decellularized tissues implants will result in fast and effective recruitment of macrophages, which in turn will induce recruitment of stem cells. These recruited stem cells will be instructed by the ECM to differentiate into cells that restore the biological activity of the implant within the treated patients. The present example demonstrates the effective recruitment of macrophages into implants injected with submicroscopic α-gal nanoparticles and the ability of the cytokines/growth factors secreted from the recruited and activated macrophages to delay necrosis of the implanted ischemic tissue. Hearts were harvested from KO mice and injected with 1 mg α-gal nanoparticles or with saline. Recipient KO mice producing the anti-Gal antibody were implanted subcutaneously with the injected hearts without connecting any blood vessels to these hearts, i.e. the implanted hearts were ischemic. Since the hearts contain only dead cells the may be viewed also as representing bioimplants containing α-gal nanoparticles. The implanted hearts were harvested after 4 weeks and subjected to histological analysis in order to determine whether the α-gal nanoparticles injected into these hearts can induce the recruitment of macrophages even if the organ is not connected to the blood circulation. FIG. 34A shows histological sections of KO mouse hearts. Implanted hearts harvested after 4 weeks show multiple recruited macrophages at the injected area despite the fact that the implants were not connected to the circulation of the recipients. At that time point the macrophages further migrate from the injection area in which they are recruited by α-gal nanoparticles into the non-injected areas of the myocardium. This migration of macrophages is further demonstrated in FIG. 34B which presents an area of the myocardium far from the injection site of α-gal nanoparticles. Although the structure of the cardiomyocytes at the non-injected area is preserved even after 4 weeks, the cardiomyocytes are dead. This is indicated by the lack of nuclei in these cardiomyocytes due to nucleases activity. The cells containing nucleus are the macrophages that migrate from the injection area where they were recruited into the myocardium. It is contemplated that these macrophages that were activated by interaction with anti-Gal coated α-gal nanoparticles recruit stem cells that will be further instructed by the ECM and by the conserved cardiomyocytes lacking nucleus to differentiate into cardiomyocytes that restore the activity of the myocardium. It should be noted that at the 4 week time point, implanted hearts that were injected with saline completely disappear from the recipient.

These observations with heart implants further imply that α-gal nanoparticles are very effective in inducing recruitment of macrophages into various injured tissues which are treated for induction of regeneration and are likely to mediate a similar effective recruitment of macrophages into decellularized organ and tissue implants, even if such implants are not connected to the circulation.

### Example 30

### Recruitment of Macrophages by α-Gal Nanoparticles into in the Large Animal Model of KO Pigs

This example provides a validation in the large animal model of KO pigs on the ability of submicroscopic α-gal nanoparticles injected into the myocardium to induce rapid recruitment of macrophages. The KO pigs were found to produce the natural anti-Gal IgG antibody already by the age of 1.5 months. Galili Xenotransplantation 20:267, 2013. Two KO pigs (3 month old) were injected into the myocardium with ˜0.2 ml α-gal nanoparticles (100 mg/ml) by using an injection catheter that was navigated into the left ventricle. The injection was in a area near the endocardium of the healthy KO pig heart. One of the pigs was euthanized after 5 days and the second after 7 days. The injected areas of the hearts were fixed in formalin and subjected to H&E staining. (A) Heart of the pig euthanized 5 days post injection displays multiple macrophages that were recruited around the injection area which is identified by the empty area in the damage myocardium. (B) Heart of the pig euthanized 7 days post injection. The recruited macrophages (cells with large oval nucleus) leave the recruitment area and start migrating away. Since the cardiomyocytes are alive and functioning, the migrating macrophages (characterized by large nuclei due to their activation) form “raws” of cells that migrate into the myocardium (×100). These findings imply that injection of α-gal nanoparticles into ischemic myocardium also will induce rapid recruitment and activation of macrophages within the ischemic myocardium.

### Example 31

### Recruitment of Macrophages into a Plasma Clot Containing α-Gal Nanoparticles

This example demonstrated the ability of a semi-solid filler containing α-gal nanoparticles to induce recruitment and activation of macrophages. Such semi-solid fillers are required for application of α-gal nanoparticles into spaces within or adjacent to internal injuries, while preventing extensive dispersion of these nanoparticles in the body. The semi-solid filler in this example is in the form of a plasma clot gel containing α-gal nanoparticles. Plasma was mixed volume/volume with submicroscopic α-gal nanoparticles (10 mg/ml) and allowed to form a clot by adding calcium chloride (CaCl₂) at a final concentration of 10 mM. The CaCl₂ induces conversion of fibrinogen to fibrin and the formation of a plasma clot. These clots were placed on excisional wounds of anti-Gal producing KO mice. Plasma clots were removed from the wounds 3 days and 6 days after the initiation of treatment. Macrophages recruited into the clot by anti-Gal/α-gal nanoparticle interaction and complement activation are clearly detected within 3 days after placing the plasma clot gel on the wound (FIG. 36A). The number of recruited macrophages greatly increases after 6 days and they seem to fill the plasma clot gel (FIG. 36B). These findings demonstrate the very effective mechanism of macrophage recruitment by complement cleavage chemotactic factors that are generated as a result of the antibody/antigen interaction between the anti-Gal antibody and α-gal nanoparticles. The activation of macrophages recruited into the plasma clot gel further resulted in secretion of cytokines/growth factors that induced rapid regeneration of the epidermis over this gel, as indicated in FIG. 36B.

These findings imply that various semi-solid fillers or gels containing α-gal nanoparticles and enabling diffusion of various proteins, such as hydrogels, fibrin glue and plasma clots will enable the recruitment of macrophages into sites in the body where they are administered. This recruitment will be the result of anti-Gal/α-gal nanoparticles interaction and complement activation, thereby generating chemotactic complement cleavage peptides. The recruited macrophages migrating into these gels will be further activated by binding of anti-Gal coated α-gal nanoparticles to Fcγ receptors on macrophages and secretion of pro-healing cytokines/growth factors that facilitate the regeneration of treated injured tissues.

### Example 32

### Recruitment of Macrophages and Angiogenesis by α-Gal Nanoparticles in Accelerated Wound Healing in the Large Animal Model of KO Pigs

The ability of submicroscopic α-gal nanoparticles to recruit and activate macrophages the induction of angiogenesis by VEGF secreted from these activated macrophages was further validated in the large animal model of GT-KO pigs. Similar to KO mice, KO pigs have a disrupted (knocked out) α1,3GT gene and thus, they lack α-gal epitopes and can produce the anti-Gal antibody (Phelps et al. Science 299:411, 2003; Yamada et al. Nature Med. 11:32, 2004; Galili Xenotransplantation, supra). Full-thickness wounds (20×20 mm and ˜3 mm deep) on the back of 3 month old GT-KO pigs were covered with dressings coated with 100 mg α-gal nanoparticles or with saline. The borders of each wound were tattooed with 8 dots prior to treatment for determining tissue contraction. Photographs were taken when wound dressings were changed every 3-4 days and wound surface area not covered by regenerating epidermis was evaluated (N=9) (Hurwitz et al. Plastic and Reconstructive Surgery, 129: 242e, 2012).

On day 7, all wounds were filled with granulation tissue. The greatest gross morphology differences were observed on day 13 (FIG. 37). Part of the surface area in saline treated wounds was covered by regenerating epidermis due to physiologic healing processes. Thus, on average, the size of the non-covered wound area was ˜25 mm² (i.e. ˜0.5×0.5 cm). However, many of the wounds treated with 100 mg α-gal nanoparticles were completely covered by regenerating epidermis (FIG. 37). On average, wounds treated with 100 mg α-gal nanoparticles had ˜90% smaller non-covered areas than saline treated wounds. Full healing of saline treated wounds was observed on day 18-20 (not shown). Thus, healing time of treated wounds was shortened by 30-40%.

Wounds were harvested from pigs euthanized on days 7 and 13. Day 7 wounds treated with 100 mg α-gal nanoparticles displayed many more macrophages infiltrating the granulation tissue in the center of the wound (FIG. 38A) compared with saline treated wounds (FIG. 38B). A much higher concentration of recruited macrophages was also observed on Day 13 in the center of the wounds treated with α-gal nanoparticles (FIG. 38C) and under the leading edge of the regenerating epidermis in these wounds (FIG. 38E), than in the corresponding sites in wounds treated with saline (FIGS. 38D and 38F respectively).

The macrophages recruited into wounds treated with α-gal nanoparticles were further inactivated and secreted VEGF as can be inferred from the extensive angiogenesis observed in α-gal nanoparticles treated wounds. This could be demonstrated in day 13 wounds viewed in the area under the leading edge of the regenerating epidermis. Wounds treated with 100 mg α-gal nanoparticles displayed multiple blood vessels and even cisterna of blood (stained red in the FIGS. 38C and 38E). In contrast, the concentration of blood vessels was much lower in saline treated wounds (FIGS. 38D and 38F), implying a much lower local secretion of VEGF in saline treated wounds than in α-gal nanoparticles treated wounds. These observations imply that anti-Gal/α-gal nanoparticles interaction in KO pig wounds results in rapid recruitment and activation of macrophages that further induce extensive angiogenesis in the treated injury sites.

### Example 33

### Recruitment of Pluripotent Stem Cells by α-Gal Nanoparticles in KO Mice

This example demonstrated in KO mice the ability of α-gal nanoparticles to recruit pluripotent stem cells after they interact with the anti-Gal antibody in PVA sponge discs implanted subcutaneously in these mice. PVA sponge discs containing 0.15 ml of a suspension of 1 mg/ml α-gal nanoparticles were implanted subcutaneously in anti-Gal producing KO mice. After 5 weeks the PVA sponge discs were retrieved, sectioned and stained with hematoxylin-eosin (H&E) (A) or with trichrome (for staining collagen blue) (B). (A) Formation of nerve fibers comprised of multiple axons could be observed in the PVA sponge discs. These nerve bundles are represented by the three organized horizontal bundles are generated from pluripotent stem cells recruited into the PVA sponge disc. In addition, blood vessels are observed in the lower left corner and upper left corner under the letter (A) indicating angiogenesis induced following the recruitment of macrophages by α-gal nanoparticles within the PVA implant (×200). (B) Stem cells recruited into the PVA sponge disc differentiate into of myotubes (four horizontal red structures of striated muscle, striation in the two upper myotubes can be observed upon magnification of the picture) and of connective tissue with secreted collagen stained blue by the trichrome staining (×200). The PVA sponge material is stained as grey in (A) and grey blue reticular material in (B). The multiple types of cells in subcutaneously implanted PVA sponge discs containing α-gal nanoparticles, including nerve cells, muscle cells and fibroblasts imply that the stem cells recruited by the activated macrophages are pluripotent/pluripotential. The blood vessels observed in the PVA sponge discs further imply that the activated macrophages induce angiogenesis within the implants. Implants lacking α-gal nanoparticles display few cells which primarily are fibroblasts and adipocytes as in FIG. 33E below.

### Example 34

### Recruitment of Stem Cells by α-Gal Nanoparticles in the Presence of Meniscus Cartilage Fragments Results in Formation of Fibrocartilage

This example describes a process in which stem cells are recruited by macrophages which were recruited by α-gal nanoparticles into PVA sponge discs in anti-Gal producing KO mice (FIG. 33). These stem cells are instructed by the ECM of a biomaterial comprised of dead tissue to differentiate into cells that regenerate the dead tissue. This process is illustrated by the formation of fibrocartilage which comprises meniscus cartilage as a result of recruitment of macrophages and stem cells by α-gal nanoparticles mixed with cartilage fragments. This process was studied in KO mice implanted subcutaneously with polyvinyl alcohol (PVA) sponge discs containing this mixture a mixture of submicroscopic α-gal nanoparticles and small (10-100 μm) meniscus cartilage fragments from homogenized tissue. These PVA sponge discs have a diameter of 10 mm and are 3 mm thick. These sponge discs contained 150 μl suspension of 1 mg/ml α-gal nanoparticles mixed with 10 mg/ml homogenate of pig meniscus cartilage fragments devoid of α-gal epitopes. Removal of α-gal epitopes was achieved prior to fragmentation by 20 h incubation at 24° C. with 100 Units/ml recombinant α-galactosidase, followed by repeated washes (Stone et al. Transplantation, 65:1577, 1998). The removal of α-gal epitopes from the cartilage fragments is necessary in order to prevent anti-Gal binding to these fragments. If such binding takes place (i.e. if the α-gal epitopes are not removed) the recruited macrophages will internalize the anti-Gal coated cartilage fragments. This process will prevent the formation of the appropriate microenvironment for the differentiation of stem cells into chondroblasts. The cartilage homogenate contained no live chondroblasts or chondrocytes.

PVA sponge discs containing α-gal nanoparticles and pig meniscus cartilage fragments devoid of α-gal epitopes were implanted under the skin, retrieved after five weeks, fixed, sectioned and stained with H&E or with trichrome (for staining collagen in blue) (FIG. 33). A section through a full size of the sponge disc is shown in FIG. 33A. The areas within the rectangles are areas of fibrocartilage formation. At a higher magnification these areas of fibrocartilage growth are stained in red by H&E (mostly due to the presence of hyaluronic acid) (FIG. 33B), and in deep blue by trichrome staining of the collagen fibers of the fibrocartilage (FIGS. 33C and 33D). The generated fibrocartilage is similar in structure to meniscus cartilage which is characterized by relatively few fibrochondrocytes (identified by their elongated nuclei) and large amount of extracellular matrix comprised of the fibrocartilage matrix secreted by these cells (FIG. 33F). In sponge discs containing meniscus fibrocartilage fragments but no α-gal nanoparticles (FIG. 33E), the de novo formation of fibrocartilage is residual. Most of the cells growing in the absence of α-gal nanoparticles form fat tissue or loose connective tissue. Normal meniscus fibrocartilage is presented in FIG. 35F in which the cells and fibrocartilage matrix have a parallel alignment. The histology of this unprocessed tissue is similar to that in FIGS. 33B, 33C and 33D with the exception that in the sponge disc the cells and fibrocartilage matrix are organized in a variety of directions because of the space constraining structure of the PVA sponge. This structure does not allow for parallel alignment of the tissue components because of the many small spaces.

These observations imply that implantation of a decellularized organ or tissue containing the ECM scaffold and containing α-gal nanoparticles may result in recruitment of macrophages that will be activated and will recruit stem cells. The recruited stem cells will be guided by the ECM to differentiate into cells that restore the biological activity of the implanted organ or tissue. These observations further imply that application of α-gal nanoparticles mixed with cartilage homogenate in the form of semi-solid filler such as, but not limited to, hydrogel, fibrin glue or plasma clot gel to cartilage defect will recruit macrophages to that site and induce activation of the recruited macrophages. These activated macrophages will recruit stem cells that will differentiate into chondroblasts that will secrete their ECM including collagen and other cartilage matrix proteins and glycosaminoglycans, resulting in repair and regeneration of the injured cartilage. Similarly, macrophages recruited and activated by the binding α-gal nanoparticles applied by semi-solid filler to damaged bone will secrete growth factors and cytokines that recruit stem cells that differentiate into osteoblasts and osteoclasts into the treated site for repair and regeneration of the damaged bone.

In the absence of the specific ECM that provides cues to the recruited stem cells to differentiate into the cells generating that ECM, the recruited stem cells may differentiate into various types of mature cells. This is demonstrated in FIG. 33 where PVA sponge discs containing α-gal nanoparticles and implanted subcutaneously for 5 weeks in anti-Gal producing GT-KO mice. Histological inspection of the implanted PVA sponge discs demonstrated the formation of nerve tissue, skeletal muscle tissue, connective tissue and blood vessels, without specific overall direction of stem cell differentiation.

In summary, the present invention provides numerous advantages over the prior art, including methods and compositions for the accelerated healing of wounds, recruitment and activation of macrophages that induce recruitment of stem cells into injured tissues and into bioimplants, repair and regeneration of injured tissues. All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in diagnostics, cell culture, and/or related fields are intended to be within the scope of the present invention.

I claim:

1. A method for inducing recruitment of macrophages into biomaterial implants for activation of said recruited macrophages to produce pro-healing cytokines and growth factors in a subject having endogenous anti-Gal antibody, comprising administering to said biomaterial composition that comprises α-gal nanoparticles, wherein
i) said α-gal nanoparticles comprises α-gal epitope having a terminal α-galactosyl and
ii) said administering is under conditions for increasing the amount of macrophages in injured internal tissue of said subject.

2. The method of claim 1, wherein said terminal α-galactosyl is selected from the group consisting of Galα1-3Gal, Galα1-2Gal, Galα1-6Gal or any α-galactose sugar units capable of binding anti-Gal antibodies.

3. The method of claim 1, wherein said α-gal epitope is free or part of a molecule selected from the group consisting of a natural or synthetic glycolipid, glycoprotein, and a glycopolymer.

4. The method of claim 1, wherein said biomaterial is a natural tissue or organ selected from the group consisting of heart, urinary bladder, gall bladder, lung, trachea, bronchi, bronchioles, alveoli, skeletal muscle, smooth muscle, connective tissue, endocrine glands, exocrine glands, ligament, cartilage, bone, nerve tissue, brain, spinal cord, blood vessels, liver, kidney, thyroid, parathyroid, pancreas, esophagus, stomach, small intestine, large intestine, ovary, testis, eye, ear, and skin.

5. The method of claim 1, wherein said biomaterial implant is comprised of collagen mixed with α-gal nanoparticles or containing α-gal nanoparticles, cartilage fragments mixed with α-gal nanoparticles or bone fragments mixed with α-gal nanoparticles.

6. The method of claim 1, wherein said biomaterial implant is dried or not dried and immersed in α-gal nanoparticles suspension for penetration of said α-gal nanoparticles into said biomaterial implant.

7. The method of claim 1, wherein said biomaterial implant organ or tissue is perfused with α-gal nanoparticles suspension in order to introduce said α-gal nanoparticles into said biomaterial.

8. The method of claim 1 wherein anti-Gal antibodies are bound to said α-gal nanoparticles.

9. The method of claim 1, wherein said applying is under conditions such that complement activation within or adjacent to said biomaterial implant is enhanced.

10. The method of claim 9, wherein said complement activation comprises production of complement fragments C5a, C4a and C3a.

11. The method of claim 1, wherein said applying is under conditions for enhancing one or both of (a) monocyte and macrophage recruitment within or adjacent to said biomaterial implant, and (b) stem cell recruitment within or adjacent to said biomaterial implant.

12. The method of claim 1, wherein said biomaterial is a synthetic biomaterial.

13. A method for inducing recruitment of macrophages into biomaterial implants for activation of said recruited macrophages to produce pro-healing cytokines and growth factors in a subject having endogenous anti-Gal antibody, comprising administering to said biomaterial composition that comprises a soluble molecules with one or more α-gal epitopes, wherein
i) said soluble molecule α-gal carrying molecule comprises α-gal epitope having a terminal α-galactosyl and
ii) said administering is under conditions for increasing the amount of macrophages in said biomaterial implant of said subject.

14. The method of claim 13, wherein said terminal α-galactosyl is selected from the group consisting of Galα1-3Gal, Galα1-2Gal, Galα1-6Gal or any α-galactose sugar units capable of binding anti-Gal antibodies.

15. The method of claim 13, wherein said α-gal epitope is free or part of a molecule selected from the group consisting of a natural or synthetic glycolipid, glycoprotein, and a glycopolymer.

16. The method of claim 13, wherein said biomaterial is a natural tissue or organ selected from the group consisting of heart, urinary bladder, gall bladder, lung, trachea bronchi, bronchioles, alveoli, skeletal muscle, smooth muscle, connective tissue, ligament, cartilage, bone, nerve tissue, brain, spinal cord, liver, kidney, thyroid, parathyroid, pancreas, esophagus, stomach, small intestine, large intestine, eye, ear, and skin.

17. The method of claim 13, wherein said biomaterial implant is comprised of collagen, cartilage fragments or bone fragments mixed with said soluble α-gal epitopes carrying molecule.

18. The method of claim 13, wherein said biomaterial implant is dried or not dried and immersed in soluble α-gal epitopes carrying molecule suspension for penetration of said soluble α-gal epitopes carrying molecule into the biomaterial implant.

19. The method of claim 13, wherein said biomaterial implant organ or tissue is perfused with soluble α-gal epitopes carrying molecules in order to introduce soluble α-gal epitopes carrying molecules into said biomaterial.

20. The method of claim 13 wherein anti-Gal antibodies are bound to said soluble α-gal epitopes carrying molecule.

21. The method of claim 13, wherein said applying is under conditions such that complement activation within or adjacent to said biomaterial implant is enhanced.

22. The method of claim 21, wherein said complement activation comprises production of complement fragments C5a, C4a and C3a.

23. The method of claim 13, wherein said applying is under conditions for enhancing one or both of (a) monocyte and macrophage recruitment within or adjacent to said biomaterial, and (b) stem cell recruitment within or adjacent to said biomaterial implant.

24. The method of claim 13, wherein said biomaterial is a synthetic biomaterial.

25. A method for inducing recruitment of macrophages into injured internal tissues and for activation of said recruited macrophages to produce pro-healing cytokines and growth factors in a subject having endogenous anti-Gal antibody, comprising administering to said injured internal tissue composition that comprises α-gal nanoparticles, wherein.
i) said α-gal nanoparticles comprises α-gal epitope having a terminal α-galactosyl and
ii) said administering under conditions that increasing the amount of macrophages in said injured internal tissue of said subject.

26. The method of claim 25, wherein said terminal α-galactosyl is selected from the group consisting of Galα1-3Gal, Galα1-2Gal, Galα1-6Gal or any α-galactose sugar units capable of binding anti-Gal antibodies.

27. The method of claim 25, wherein said α-gal epitope is free or part of a molecule selected from the group consisting of a natural or synthetic glycolipid, glycoprotein, and a glycopolymer.

28. The method of claim 25, wherein said α-gal nanoparticles are applied to injured internal tissues including: heart muscle, skeletal muscle, smooth muscle, connective tissue, ligament, bone, nerve tissue, brain, spinal cord, blood vessels, endocrine glands, exocrine glands, liver, kidney, gall bladder, thyroid, parathyroid, pancreas, esophagus, stomach, small intestine, large intestine, lung, trachea, bronchi, bronchioles, alveoli, eye, ear, ovary, testis, urinary bladder, skin.

29. The method of claim 25, wherein said α-gal nanoparticles are applied to injured or severed fingers, toes, arms and feet.

30. The method of claim 25, wherein said preparation is part of an injury care device selected from the group consisting of injections, adhesive bands, compression bandages, gels, semi-permeable films, plasma clots, fibrin clots, water, solutions, suspensions, emulsions, creams, ointments, aerosol sprays, collagen containing substances, stabilizers, sponges, drops, matrix-forming substances, foams or dried preparation.

31. The method of claim 25, wherein said applying is under conditions such that complement activation within or adjacent to said injured tissue is enhanced.

32. The method of claim 31, wherein said complement activation comprises production of complement cleavage chemotactic peptides including C5a, C4a and C3a.

33. The method of claim 25, wherein said applying is under conditions for enhancing one or both of (a) monocyte and macrophage recruitment within or adjacent to said injured tissue, and (b) stem cell recruitment within or adjacent to said injured tissue.

34. The method of claim 25, wherein said applying is under conditions such that injury healing and tissue repair and regeneration is induced or accelerated.

35. A method for inducing recruitment of macrophages into injured internal tissues and for activation of said recruited macrophages to produce pro-healing cytokines and growth factors in a subject having endogenous anti-Gal antibody, comprising administering to said injured internal tissue composition that comprises a soluble molecules with one or more α-gal epitopes, wherein.
i) said soluble molecule α-gal carrying molecule comprises α-gal epitope having a terminal α-galactosyl and
ii) said administering under conditions that increasing the amount of macrophages in said injured internal tissue of said subject.

36. The method of claim 35, wherein said terminal α-galactosyl is selected from the group consisting of Galα1-3Gal, Galα1-2Gal, Galα1-6Gal or any α-galactose sugar units capable of binding anti-Gal antibodies.

37. The method of claim 35, wherein said α-gal epitope is free or part of a molecule selected from the group consisting of a natural or synthetic glycolipid, glycoprotein, and a glycopolymer.

38. The method of claim 35 wherein said soluble α-gal epitope carrying molecules are applied to injured internal tissues including: heart muscle, skeletal muscle, smooth muscle, connective tissue, ligament, bone, nerve tissue, brain, spinal cord, liver, kidney, thyroid, parathyroid, pancreas, esophagus, stomach, small intestine, large intestine, lung, trachea, bronchioles, alveoli, eye, ear, glands, blood vessels, ovary, testis and skin.

39. The method of claim 35, wherein said soluble α-gal epitope carrying molecules are applied to injured or severed fingers, toes, arms and feet.

40. The method of claim 35, wherein said soluble α-gal epitope carrying molecules are part of a injury care device selected from the group consisting of injections, adhesive bands, compression bandages, gels, semi-permeable films, plasma clots, fibrin clots, water, solutions, suspensions, emulsions, creams, ointments, aerosol sprays, collagen containing substances, stabilizers, sponges, drops, matrix-forming substances, foams or dried preparation.

41. The method of claim 35, wherein said applying is under conditions such that complement activation within or adjacent to said injured tissue is enhanced.

42. The method of claim 41, wherein said complement activation comprises production of complement fragments C5a, C4a and C3a.

43. The method of claim 35, wherein said applying is under conditions for enhancing one or both of (a) monocyte and macrophage recruitment within or adjacent to said injured tissue, and (b) stem cell recruitment within or adjacent to said injured tissue.

44. The method of claim 35, wherein said applying is under conditions such that injury healing and tissue repair and regeneration is accelerated.
